(19) United States
(12) Patent Application Publication
(10) Pub. No.: US 2026/0138961 A1
(43) Pub. Date: May 21, 2026

(54) MODULATORS OF MITOCHONDRIAL DNA REPLICATION

(71) Applicant: Pretzel Therapeutics, Inc., Waltham, MA (US)

(72) Inventors: Andrew Griffin, L'Île-Bizard, Quebec (CA); Andrew McKenzie, Belmont, MA (US); Paul S. Charifson, Carrabassett Valley, ME (US); Simon Giroux, Cambridge, MA (US); Jeremy Green, Waltham, MA (US)

(73) Assignee: Pretzel Therapeutics, Inc., Waltham, MA (US)

(21) Appl. No.: 19/141,633

(22) PCT Filed: Dec. 20, 2023
(86) PCT No.: PCT/US2023/085141

§ 371 Date: Jun. 20, 2025

Related U.S. Application Data
(60) Provisional application No. 63/434,267, filed on Dec. 21, 2022.
(60) Provisional application No. 63/434,276, filed on Dec. 21, 2022.

(51) Int. Cl. C07D 277/28; A61K 31/415; A61K 31/4164; A61K 31/426; C07D 231/12; C07D 233/64

(52) U.S. Cl. CPC C07D 277/28; A61K 31/415; A61K 31/4164; A61K 31/426; C07D 231/12; C07D 233/64

(57) ABSTRACT

The present invention provides novel sulfonamide compounds that are modulators of POLγ for treating various diseases such as cancer and others associated with metabolic disorders and mitochondrial dysfunction.

### CROSS-REFERENCE TO RELATED APPLICATIONS

This application is the National Stage entry under 35 U.S.C. § 371 of International Application No. PCT/US2023/085141, filed on Dec. 20, 2023, published on Jun. 27, 2024 under Publication Number WO 2024/137813 A1, which claims priority from U.S. Provisional Application No. 63/434,267, filed Dec. 21, 2022 and U.S. Provisional Application No. 63/434,276 filed Dec. 21, 2022, all of the applications identified above are incorporated herein by reference in their entirety.

### FIELD OF THE INVENTION

Embodiments of the invention relate to novel DNA polymerase γ (POLy) modulators, their prodrugs, their pharmaceutically acceptable salts, and pharmaceutical compositions thereof. Embodiments of the invention also relate to methods of using such compounds and compositions, including to modulate (e.g., inhibit or promote) POLy, and to treat various mitochondrial diseases, neurodegenerative and metabolic disorders, cancer, inflammation, infectious diseases, and also disorders related to aging.

### BACKGROUND OF THE INVENTION

Human mitochondria contain multiple copies of a circular, double-stranded (ds) DNA genome (mtDNA), and a dedicated DNA replication machinery is required for maintaining the mitochondrial genome. DNA polymerase γ (POLγ) is the replicative polymerase essential for maintaining the mtDNA. POLγ is a heterotrimer in human cells with one catalytic subunit (POLγA) and two accessory subunits (POLγB). POLγA belongs to the family A DNA polymerases. POLγA is 1239 amino acids in length and consists of three distinct regions: an N-terminal exonuclease domain connected by a linker domain to the C-terminal polymerase domain. The accessory POLγB is 485 amino acids in length and the crystal structures of both mouse and human POLγB have revealed the protein as a dimer with high similarities to aminoacyl tRNA synthetases. POLγB acts as a processivity factor, which increases the affinity of the polymerase for DNA and promotes tighter nucleotide binding, thereby increasing the polymerase rate. See, e.g., Gustafsson, C. M., et al., “Maintenance and expression of mammalian mitochondrial DNA,” Annu. Rev. Biochem., 85:133-160 (2016) (Gustafsson 2016) and Farge, G., et al., “The accessory subunit B of DNA polymerase γ is required for mitochondrial replisome function,” Nucleic Acids Res., 35:902-911 (2007).

A primary biological role of POLγ is to replicate the mitochondrial genome. However, POLγ cannot replicate the double stranded (ds) mtDNA alone. It acts together with a DNA helicase (TWINKLE) and the mitochondrial single-stranded DNA-binding protein (mtSSB). TWINKLE is 684 amino acids in length and forms a hexamer in solution. TWINKLE travels in front of POLγ during mtDNA replication, unwinding the dsDNA and creating a single-stranded (ss) DNA template that POLγ can utilize. The mtSSB is 148 amino acids in length and is active as a tetramer. It binds to ssDNA, protects this DNA against nucleases, and prevents secondary structure formation so POLγ can use ssDNA as a template to synthesize dsDNA. The mtSSB enhances mtDNA synthesis by increasing the processivity of POLγ and also stimulates TWINKLE's helicase activity. POLγ cannot initiate DNA synthesis de novo, as it needs a short RNA primer to initiate DNA synthesis. In mitochondria, the mitochondrial RNA polymerase (POLRMT) has a dual function; it acts as an RNA polymerase involved in mtDNA transcription but it also synthesizes the primers needed to initiate mtDNA replication from mitochondrial origins of replication. See, e.g., Gustafsson 2016.

The mitochondrial genome encodes subunits of the oxidative phosphorylation (OXPHOS) system. The OXPHOS system is composed of four respiratory chain complexes, which are responsible for electron transport and generation of the proton gradient across the mitochondrial inner membrane. ATP synthase uses this proton gradient to produce ATP. See, e.g., id. The biogenesis of the OXPHOS system is under dual genetic control and requires the concerted expression of nuclear DNA and mtDNA encoded genes. Mitochondria contain multiple copies of ds mtDNA, which encodes 2 ribosomal RNAs (mt-rRNAs), 22 transfer RNAs (mt-tRNAs), and 11 messenger RNAs (mt-mRNAs) producing 13 protein subunits of OXPHOS complexes I, III, IV, and ATP synthase (sometime referred to as complex V). The biogenesis of the OXPHOS system is critically dependent on the mtDNA-encoded subunits as they typically have key catalytic roles or are core subunits for OXPHOS assembly. Similar to the nuclear genome, expression of mammalian mtDNA requires several essential steps, including genome maintenance, replication, transcription, RNA maturation, and translation. All proteins involved in these processes are encoded in the nuclear genome, translated in the cytosol, and imported into the mitochondrial network. It is estimated that approximately one quarter of the ˜1200 nucleus-encoded mitochondrial proteins are devoted to the control of mtDNA gene expression in mammals. See, e.g., Shokolenko, I. N., et al., “Mitochondrial transcription in mammalian cells,” Front. Biosci. (Landmark Ed.), 22:835-853 (2017). POLγ is required for mtDNA synthesis and is thus essential for biogenesis of the OXPHOS system, resulting in ATP production. ATP production is in turn vital for energy homeostasis in the cell.

Mutations affecting POLγ are among the most frequent causes of mitochondrial disease. More than 300 disease-causing variants have been identified in POLγ, causing a broad clinical spectrum of neurodegenerative and mitochondrial diseases such as Alpers syndrome, stroke-like episodes, and chronic progressive external ophthalmoplegia. Pathogenic variants in the gene encoding POLγA, namely POLG, are now known to cause a spectrum of overlapping phenotypes. See, e.g., Rahman, S., et al., “POLG-related disorders and their neurological manifestations,” Nat. Rev. Neurol., 15:40-52 (2019) (Rahman 2019). POLγ has also been linked to more common disease such as Parkinson's disease and to normal aging. See, e.g., Luoma, P., et al., “Mitochondrial DNA polymerase gamma variants in idiopathic sporadic Parkinson disease,” Neurology, 69:1152-59 (2007) and Wallace, D. C., et al., “A mitochondrial paradigm of metabolic and degenerative diseases, aging, and cancer: a dawn for evolutionary medicine,” Annu. Rev. Genet., 39:359-407 (2005). These POLγ mutations are linked to the accumulation of damaged mtDNA, including multiple deletions, but can also lead to loss of mtDNA (depletion). Many disease-causing variants of POLγ are associated with decreased replication processivity of the mtDNA replication machinery, leading to replication stalling.

Clinical characteristics of POLγ-related disorders include, but are not limited to, Alpers-Huttenlocher syndrome (AHS), Childhood myocerebrohepatopathy spectrum (MCHS), Myoclonic epilepsy myopathy sensory ataxia (MEMSA), the ataxia neuropathy spectrum (ANS), Autosomal recessive progressive external ophthalmoplegia (arPEO), and Autosomal dominant progressive external ophthalmoplegia (adPEO). See Cohen B. H., Chinnery P. F., and Copeland W. C., POLG-Related Disorders. 2010 Mar. 16 [Updated 2018 Mar. 1]. In: Adam M. P., Ardinger H. H., Pagon R. A., et al., editors. GeneReviews [Internet]. Seattle (WA): University of Washington, Seattle; 1993-2022.

Small molecule compounds may ameliorate defects caused by mutations in other components of the mtDNA replication machinery, such as mutations in the genes encoding the helicase TWINKLE, mtSSB, or POLRMT. Compounds that stimulate the synthesis ability of POLγ may also be useful for treatment of mtDNA depletion syndromes caused by lower levels of nucleotides, e.g., mutations in genes such as TP, TK2, DGUOK, RRM2B, SUCLA2, and SUCLG1. See, e.g., Rahman 2019.

Changed levels of mtDNA or mtDNA mutations (including deletions) have also been implicated in more common disorders (such as Type-2 diabetes, Alzheimer's disease, and Parkinson's disease) and aging. See, e.g., Filograna, R., et al., “Mitochondrial DNA copy number in human disease: the more the better?” FEBS Lett., 595:976-1002 (2021). With respect to aging, most data show a reduction in mtDNA levels and increased mtDNA deletions in the older population. See, e.g., Scott, A. L., et al., “Ultrasensitive deletion detection links mitochondrial DNA replication, disease, and aging,” Genome Biology, 21:248 (2020).

In the brain, the dopaminergic substantia nigra is particularly susceptible to somatic mtDNA deletions, which accumulate there at substantially higher levels compared with other brainstem nuclei, deep grey structures, or the cerebral and cerebellar cortex. This predilection has led to the hypothesis that mtDNA damage plays a role in the pathogenesis of Parkinson's disease, where neurodegeneration of the substantia nigra is the main pathological hallmark and is widely accepted as the cause of the cardinal clinical features. See, e.g., Bender, A. et al., “High levels of mitochondrial DNA deletions in substantia nigra neurons in aging and Parkinson disease,” Nat. Genet., 38:515-517 (2006). There is also an increased risk of Parkinson's disease associated with the genetic variation in genes encoding key factors of mtDNA maintenance, such as POLγ and TFAM. See, e.g., Luoma, P. T., et al., “Mitochondrial DNA polymerase gamma variants in idiopathic sporadic Parkinson disease,” Neurology, 69:1152-1159 (2007) and Belin, A. C., et al., “Association study of two genetic variants in mitochondrial transcription factor A (TFAM) in Alzheimer's and Parkinson's disease,” Neurosci. Lett., 420:257-262 (2007). In addition, it has been reported that in dopaminergic substantia nigra neurons of healthy individuals, mtDNA copy number increases with age, maintaining the pool of wild-type mtDNA population in spite of accumulating deletions. This upregulation fails to occur in individuals with Parkinson's disease, however, resulting in depletion of the wild-type mtDNA population. See, e.g., Dölle, C., et al., “Defective mitochondrial DNA homeostasis in the substantia nigra in Parkinson disease,” Nat. Commun., Nov. 22; 7:13548. doi: 10.1038/ncomms13548 (2016). Combined, the existing literature suggests that dysregulation of mtDNA homeostasis is a key process in the pathogenesis of neuronal loss in Parkinson's disease.

There is also correlation between neurodegeneration in both Alzheimer's disease and amyotrophic lateral sclerosis (ALS) with reduced levels of mtDNA and increased mutations in mtDNA. See, e.g., Corral-Debrinski, M., et al., “Marked changes in mitochondrial DNA deletion levels in Alzheimer brains,” Genomics, 23(2):471-476 (1994). In sporadic ALS skeletal muscle there is a reduction in activity of respiratory chain complexes with subunits encoded by mtDNA that are associated with reduced mtDNA content. See, e.g., Vielhaber, S., et al., “Mitochondrial DNA abnormalities in skeletal muscle of patients with sporadic amyotrophic lateral sclerosis,” Brain, 123:1339-1348 (2000). Small molecular compounds that can stimulate the DNA synthesis ability of POLγ may be useful in the treatment of neurodegenerative disorders.

In view of the numerous and varied roles of POLγ, the need exists for potent and specific modulators of POLγ.

### SUMMARY OF THE INVENTION

Provided are compounds, pharmaceutically acceptable salts of the compounds, and prodrugs of the compounds; pharmaceutical compositions comprising the compounds or their salts or prodrugs; and methods of using the compounds, salts of the compounds, prodrugs of the compounds, or pharmaceutical compositions of the compounds, their salts, or their prodrugs to treat mitochondrial diseases, neurodegenerative and metabolic disorders, cancer, inflammation, infectious diseases, and also disorders related to aging. The compounds and their pharmaceutically acceptable salts are particularly useful as modulators of POLγ.

In one embodiment, the present invention is directed to a compound, a prodrug thereof, or a pharmaceutically acceptable salt thereof, represented by formula (I):

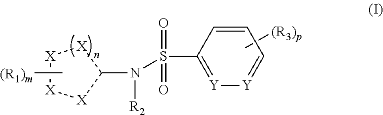

wherein:
dashed lines are independently single or double bonds;
R¹ is selected from the group consisting of:
   C₁-C₄ alkyl,
   halogen,
   C(O)-phenyl,
   C(O)—NR⁴R⁵,
   C(O)-pyrrolidine optionally substituted with one or more C₁-C₄ alkyl,
   tetrahydropyran optionally substituted with one or more C₁-C₄ alkyl,
   phenyl optionally substituted with one or more C₁-C₄ alkyl or —OR⁶,
   pyridine optionally substituted with one or more C₁-C₄ alkyl,
   thiazole optionally substituted with a group selected from the group consisting of:
      C₁-C₄ alkyl,
      C₃-C₆ cycloalkyl,
      R⁶—OH,
      R⁶—NR⁶R⁶,
      CN,
      C(O)—OH,
      C(O)—OR⁶,
      C(O)—NH₂,
      C(O)—NHR⁶,
      C(O)—NR⁶R⁶,
      tetrazole optionally substituted with one or more C₁-C₄ alkyl,
   oxazole optionally substituted with one or more C₁-C₄ alkyl or C₃-C₇ cycloalkyl,
   pyrazole optionally substituted with one or more C₁-C₄ alkyl or C₃-C₇ cycloalkyl,
   imidazole optionally substituted with one or more C₁-C₄ alkyl or C₃-C₇ cycloalkyl, wherein the C₁-C₄ alkyl is optionally substituted with phenyl, and
   piperidine optionally substituted with one or more of C₁-C₄ alkyl or C(O)—R⁶,
or two R¹ are joined together to form a 6-membered aryl ring, a 5-membered saturated heterocyclic ring, or a 5-membered unsaturated heterocyclic ring, wherein the aryl ring is optionally substituted with one or more of C₁-C₄ alkyl or one or more halogen, and wherein either heterocyclic ring optionally contains a second heteroatom selected from O and S, and either heterocyclic ring is optionally substituted with one or more groups selected from the group consisting of C₁-C₄ alkyl, phenyl, and oxo,
or two R are joined together to form a phenyl group that is optionally substituted with one or more of C₁-C₄ alkyl or halogen;
R² is hydrogen or C₁-C₄ alkyl;
R³ is selected from the group consisting of C₁-C₄ alkyl, C₁-C₄ alkoxy, C₁-C₄ trihaloalkyl, CN, and halogen,
or two R³ are joined together to form a 5- to 6-membered unsaturated heterocyclic ring that optionally contains a second heteroatom selected from N and S;
R⁴ and R⁵ are each independently selected from the group consisting of H, C₁-C₄ alkyl, 4- to 6-membered heterocyclic ring containing O, 4- to 6-membered cycloalkyl, and 4to 6-membered heteroaryl, wherein the C₁-C₄ alkyl is optionally substituted with a group selected from the group consisting of C₃-C₇ cycloalkyl, OH, OR⁶, phenyl, C(O)—NH₂, and C(O)—NR⁶R⁶,
or R⁴ and R⁵ together with the nitrogen atom to which they are joined form a 5- to 7-membered heterocyclic ring optionally containing a second heteroatom selected from N, O, and S, wherein the heterocyclic ring is optionally substituted with one or more groups independently selected from the group consisting of C₁-C₄ alkyl, C₃-C₇ cycloalkyl, C₁-C₄ alkoxy, halogen, hydroxy, NH₂, NHR⁶, NR⁶R⁶, cyano, C(O)—R⁶, C(O)NH₂, wherein the C₁-C₄ alkyl is optionally substituted with OH or OR⁶,
or R⁴ and R⁵ together with the nitrogen atom to which they are joined form a 7- to 9-membered heterobicyclic ring optionally containing one or two additional N atoms, and optionally substituted with C₁-C₄ alkyl;
R⁶ is C₁-C₄ alkyl;
X is selected from the group consisting of C, CH, CH₂, O, N, NR, and S, as allowed by valency;
Y is C, CH or N, as allowed by valency;
m is 1-2;
n is 1-2; and
p is 0-3.

Further embodiments of the present invention are compounds of the invention (that is, compounds of formula (I)), their pharmaceutically acceptable salts, or prodrugs of the compounds wherein one or more hydrogen is substituted with a deuterium atom.

Additional embodiments of the invention are pharmaceutical compositions comprising a compound of the invention, a pharmaceutically acceptable salt thereof, or a prodrug thereof and one or more pharmaceutically acceptable excipients.

Another embodiment of the invention are methods of using compounds of formula (I) for the modulating POLγ. Compounds of formula (I) can be used for the modulation of POLγ. Compounds of formula (I) can be used for the modulation of POLγ-exonuclease. Compounds of formula (I) can be used for the modulation of wild type and mutant POLγ polymerase in mammalian cells.

Further embodiments of the invention are methods of treating a disease, such methods comprising administering to a subject in need thereof a therapeutically effective amount of a compound of the invention, a prodrug thereof, or a pharmaceutically acceptable salt thereof. In some embodiments, the disease is selected from the group consisting of mitochondrial diseases, neurodegenerative and metabolic disorders, cancer, inflammation, infectious diseases, and also disorders related to aging.

Additional embodiments of the invention are methods of treating neurodegenerative disorders and metabolic disorders, such as those identified in Bonekamp, N. A. et al., “Small-molecule inhibitors of human mitochondrial DNA transcription,” Nature, 588:712-716 (2020), Filograna, R. et al., “Mitochondrial DNA copy number in human disease: the more the better?” FEBS Lett., 595:976-1002 (2021), Wrendenber, A. et al., “Respiratory chain dysfunction in skeletal muscle does not cause insulin resistance,” Biochem. Biophys. Res. Comm., 350:202-207 (2006), Pospililik, J. A. et al., “Targeted deletion of AIF decreases mitochondrial oxidative phosphorylation and protects from obesity and diabetes,” Cell, 131:476-91 (2007), and PCT Published International Publication No. WO 2019/057821 A1 and references therein.

### DETAILED DESCRIPTION OF THE INVENTION

Modulators of POLγ are useful in compositions and methods suitable for treating many disorders, such as mitochondrial diseases, neurodegenerative and metabolic disorders, cancer, inflammation, infectious diseases, and also disorders related to aging. Provided herein are compounds of formula (I), pharmaceutically acceptable salts thereof, prodrugs thereof, and pharmaceutical compositions comprising such compounds, their salts, or their prodrugs that are useful in treating a condition or disease, such as mitochondrial diseases, neurodegenerative and metabolic disorders, cancer, inflammation, infectious diseases, and also disorders related to aging.

Neurodegenerative disorders include, but are not limited to, Alzheimer's disease, amyotrophic lateral sclerosis, Friedreich ataxia, Huntington's disease, Lewy body disease, Parkinson's disease, and spinal muscular atrophy.

Metabolic disorders include, but are not limited to, familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), and Niemann-Pick.

Mitochondrial disorders include, but are not limited to, mitochondrial myopathy, diabetes mellitus and deafness (DAD), chronic progressive external ophthalmoplegia (CPEO), Kearns-Sayre syndrome (KSS), Leber hereditary optic neuropathy (LHON), mitochondrial encephalomyopathy with lactic acidosis and stroke-like episodes (MELAS), myoclonic epilepsy with ragged-red fibers (MERRF), neurogenic weakness with ataxia and retinitis pigmentosa (NARP), Pearson syndrome, Leigh syndrome, subacute necrotizing encephalomyelopathy, neuropathy, ataxia, retinitis pigmentosa, and ptosis (NARP), myoneurogenic gastrointestinal encephalopathy (MNGIE), and mitochondrial DNA depletion syndrome. See, e.g., Chinnery, P. F., “Primary Mitochondrial Disorders Overview,” Jun. 8, 2000 [Updated Jul. 29, 2021]. In: Adam, M. P., Everman, D. B., Mirzaa, G. M., et al., editors. GeneReviews [Internet]. Seattle (WA): University of Washington, Seattle; 1993-2022.

Diseases related to aging include, but are not limited to, cardiovascular disease, cancer, arthritis, dementia, cataract, osteoporosis, diabetes, hypertension, and Alzheimer's disease.

### Definitions

The term “heteroatom” as used herein refers to oxygen (O), sulfur (S), nitrogen (N), phosphorus (P), silicon (Si), or selenium (Se) and includes oxidized forms of these atoms. Furthermore, nitrogen may be quaternized or may be present with hydrogen (NH).

The term “alkyl” as used herein refers to both branched- and straight-chain saturated aliphatic hydrocarbon groups having the specified number of carbon atoms in a specified range. For example the term “C₁-C₇ alkyl” means linear or branched chain alkyl groups, including all possible isomers, having 1, 2, 3, 4, 5, 6, or 7 carbon atoms. Furthermore, alkyl groups allow for substituents to be located on any of the carbon atoms. For example, a substituted C₃ alkyl group allows for the substituent to be located on any one, or more, of the three carbon atoms.

The term “alkoxy” or “alkoxyl” as used herein refers to an —O-alkyl group. For example, the term “C₁-C₄ alkoxyl” means —O—C₁-C₄ alkyl. In certain examples, alkoxy may be represented by “OR” wherein R is an optionally substituted alkyl group. Examples of alkoxyl include methoxyl, ethoxyl, propoxyl (e.g., n-propoxyl and isopropoxyl), and the like.

The term “halogen” or “halo” as used herein refers any of the six nonmetallic elements that constitute Group 17 (Group VIIa) of the periodic table. The halogen elements include, but are not limited to, fluorine or fluoro (F), chlorine or chloro (Cl), bromine or bromo (Br), and iodine or iodo (I).

The term “cycloalkyl” as used herein refers to a completely saturated or partially unsaturated cyclic group that includes monocyclic or multicyclic ring system. In certain examples, cycloalkyl groups contain 3 to 14 carbon atoms in the ring system (“C₄-C₁₄ cycloalkyl”). In certain examples, each of the cycloalkyl rings may contain one or more double or triple bonds. Examples of cycloalkyl include, without limitation, cyclopropyl, cyclobutyl, cyclopentyl, cyclohexyl, bicyclo[2.2.0]hexane, octahydro-1H-indene, decahydronaphthalene, and tetradecahydroanthracene.

The term “heterocyclyl” refers to a cycloalkyl group, as described above, in which at least one or more heteroatoms are present in a ring that includes, but is not limited to, nitrogen (N), oxygen (O), and sulfur (S) atoms, wherein the heteroatom may be oxidized, e.g., NO, SO, SO₂, SO₄, and the N may be quaternized. In certain examples, heterocyclyl groups contain 3 to 14 atoms in the ring system (“3- to 14-membered heterocyclyl”). Heterocyclyl can be attached to the core structure via a ring carbon atom or a ring heteroatom. Typical monocyclic heterocyclyl rings include, but are not limited to, azetidinyl, pyrrolidyl, oxetanyl, pyrazolinyl, imidazolinyl, imidazolidinyl, oxazolidinyl, isoxazolidinyl, thiazolidinyl, isothiazolidinyl, tetrahydrofuryl, piperidyl, piperazinyl, hexahydroazepinyl, 4-piperidonyl, tetrahydropyranyl, morpholinyl, thiomorpholinyl, 1,3-dioxanyl, and tetrahydro-1,1-dioxythienyl.

The term “aryl” as used herein refers to a monocyclic or multicyclic ring system having the characteristics of a conjugated pi-electron system. In certain examples, aryl groups contain 4 to 14 carbon atoms in the ring system (“C₄-C₁₄ aryl”). Fused aryl groups may include an aryl ring (e.g., a phenyl ring) fused to another aryl ring. Examples, without limitation, of aryl groups include phenyl, biphenyl, naphthalenyl, anthracenyl, and phenantherenyl.

The term “heteroaryl” as used herein refers to an aryl group, as described above, containing one or more heteroatoms that includes, but is not limited to, N, O, and S atoms, wherein the heteroatom may be oxidized, e.g., NO, SO, SO₂, SO₄, and the N may be quaternized. In certain examples, heteroaryl groups contain 4 to 14 atoms in the ring system (“4- to 14-membered heteroaryl”). Examples, without limitation, include pyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., 1,2,3-, 1,2,4-, 1,2,5-(furazanyl), or 1,3,4-isomers), oxatriazolyl, thiazolyl, isothiazolyl, thiadiazolyl, benzofuran, benzothiazolyl, benzimidazolyl, benzoxazolyl, benzisoxazolyl, benzothiazolyl, benzoxadiazolyl, benzpyrazinyl, indolyl, indazolyl, indolizinyl, pteridinyl, purinyl, phthalazinyl, quinazolinyl, quinolinyl, and quinolizinyl.

The term “aryl-cycloalkyl” as used herein refers to an aryl group, as defined above, that is fused to a cycloalkyl group, as defined above, by any two or more atoms in the ring. In certain examples, aryl-cycloalkyl groups contain 6 to 14 atoms in the ring system (“6to 14-membered aryl-cycloalkyl”). Aryl-cycloalkyl can be attached to the core structure via a ring carbon atom on the aryl ring or on the cycloalkyl ring. Examples of aryl-cycloalkyl include, but are not limited to, indane, indene, tetrahydronaphthalene, 1,4-dihydronaphthalene, and bicyclo[4.2.0]octa-1,3,5-triene.

The term “heteroaryl-cycloalkyl” as used herein refers to a heteroaryl group, as defined above, which is fused to a cycloalkyl group, as defined above, by any two or more atoms in the ring. In certain examples, heteroaryl-cycloalkyl groups contain 6 to 14 atoms in the ring system (“6- to 14-membered heteroaryl-cycloalkyl”). Heteroaryl-cycloalkyl can be attached to the core structure via a ring carbon atom or heteroatom on the aryl ring or via a carbon atom on the cycloalkyl ring. Examples of heteroaryl-cycloalkyl include, but are not limited to, 1,4,5,6-tetrahydrocyclopenta[b]pyrrole, 4,5,6,7-tetrahydro-1H-indole, 2,3-cyclopentenopyridine, and 5,6,7,8-tetrahydroquinoline.

The term “aryl-heterocyclyl” as used herein refers to an aryl group, as defined above, which is fused to a heterocyclyl group, as defined above, by any two or more atoms in the ring. In certain examples, aryl-heterocyclyl groups contain 6 to 14 atoms in the ring system (“6- to 14-membered aryl-heterocyclyl”). Aryl-heterocyclyl can be attached to the core structure via a ring carbon atom on the aryl ring or a ring carbon atom or heteroatom on the heterocyclyl ring. Examples of aryl-heterocyclyl include, but are not limited to, benzopyran, chromane, 4H-chromene, isochromane, thiochromane, isothiochromane, 1,2,3,4-tetrahydroquinoline, 1,2,3,4-tetrahydroisoquinoline, 2,3-dihydrobenzofuran, 1,3-dihydroisobenzofuran, 2,3-dihydrobenzo[b]thiophene, 1,3-dihydrobenzo[c]thiophene, indoline, isoindoline, and benzdioxane.

The term “heteroaryl-heterocyclyl” as used herein refers to a heteroaryl group, as defined above, which is fused to a heterocyclyl group, as defined above, by any two or more atoms in the ring. In certain examples, heteroaryl-heterocyclyl groups contain 6 to 14 atoms in the ring system (“6- to 14-membered heteroaryl-heterocyclyl”). Heteroaryl-heterocyclyl can be attached to the core structure via a ring carbon atom or heteroatom on the aryl ring or a ring carbon atom or heteroatom on the heterocyclyl ring. Examples of aryl-heterocyclyl include, but are not limited to 2,3-dihydro-1λ²,6λ²-pyrrolo[2,3-b]pyrrole, 4,7-dihydro-5H-1λ²,6λ²-pyrrolo[2,3-c]pyridine, 5,7-dihydro-6λ²-pyrrolo[3,4-b]pyridine, and 3,4-dihydro-2H-1λ²-1,8-naphthyridine.

The term “optionally substituted” or “optional substituents” as used herein means that the groups are either unsubstituted or substituted with one or more of the substituents specified. When the groups are substituted with more than one substituent, the substituents may be the same or different. Furthermore, when using the terms “independently,” “independently are,” and “independently selected from” means that the groups may be the same or different.

The term “deuterium” as used herein refers to an isotope of hydrogen that has one proton and one neutron in its nucleus and that has twice the mass of ordinary hydrogen. Deuterium herein is represented by the symbol “D”.

The term “deuterated” by itself or used to modify a compound or group as used herein refers to the presence of at least one deuterium atom attached to carbon. For example, the term “deuterated compound” refers to a compound which contains one or more carbon-bound deuterium(s). In a deuterated compound of the present invention, when a particular position is designated as having deuterium, it is understood that the abundance of deuterium at that position is substantially greater than the natural abundance of deuterium, which is about 0.015%.

The term “undeuterated” or “non-deuterated” as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as “H” or “hydrogen”, the position is understood to have hydrogen at its natural abundance isotopic composition.

The term “isotopic enrichment factor” as used herein refers to the ratio between the isotope abundance and the natural abundance of a specified isotope.

The term “isotopologue” as used herein refers to a species in which the chemical structure differs from a specific compound of the invention only in the isotopic composition thereof.

The term “substantially free of other stereoisomers” as used herein means less than 10% of other stereoisomers, preferably less than 5% of other stereoisomers, more preferably less than 2% of other stereoisomers and most preferably less than 1% of other stereoisomers are present.

The term “pharmaceutically acceptable salt” as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to, salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with, but not limited to, alkali metal salts, alkaline earth metal salts or ammonium salts. Examples of such salts include, but are not limited to, sodium salts, potassium salts, calcium salts, magnesium salts, or salts with ammonia or organic amines such as, for example, ethylamine, ethanolamine, triethanolamine, or amino acids. Additional examples of such salts can be found in Stahl, P. H. et al., Handbook of Pharmaceutical Salts: Properties, Selection, and Use, 2^{nd} Revised Edition, Wiley, 2011.

The term “prodrug” as used herein refers to derivatives of compounds of the invention which may have reduced pharmacological activity, but can, when administered to a patient, be converted into the inventive compounds. Design and use of prodrugs may be found in “Pro-drugs as Novel Delivery Systems,” Vol. 14, ACS Symposium Series (Higuchi, T., and Stella, W.) and “Bioreversible Carriers in Drug Design,” Pergamon Press, 1987 (ed. Roche E. B., American Pharmaceutical Association), the disclosures of which are incorporated herein by reference in their entireties. Prodrugs in accordance with the invention can, for example, be produced by replacing appropriate functionalities present in the inventive compounds with certain moieties known to those skilled in the art as ‘pro-moieties’ as described, for example, in “Design of Prodrugs” by Bundgaard H., (Elsevier, 1985), the disclosure of which is incorporated herein by reference in its entirety. Some non-limiting examples of prodrugs in accordance with the invention include: (i) where the compound contains a carboxylic acid functionality —(COOH), an ester thereof, for example, replacement of the hydrogen with (C₁-C₇)alkyl; (ii) where the compound contains an alcohol functionality (—OH), an ether thereof, for example, replacement of the hydrogen with (C₁-C₇)alkanoyloxymethyl, or with a phosphate ether group; and (iii) where the compound contains a primary or secondary amino functionality (—NH₂ or —NHR, where R is not H), an amide thereof, for example, replacement of one or both hydrogens with C₁-C₇ alkanoyl. Further examples of replacement groups in accordance with the foregoing examples and examples of other prodrug types may be found in the aforementioned references.

The terms “treatment”, “treating” and “treat” as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term “therapeutically effective amount” as used herein refers to that amount of compound of the invention that will elicit the biological or medical response of a tissue, system, animal, or human that is being sought by a researcher, veterinarian, medical doctor, or other. As will be recognized by a person of ordinary skill in the art, a therapeutically effective amount of the compounds of the invention will vary and will depend on the diseases treated, the severity of the disease, the route of administration, and the gender, age, and general health condition of the subject to whom the compound is being administered. The therapeutically effective amount may be administered as a single dose once a day, or as split doses administered multiple (e.g., two, three, or four) times a day. The therapeutically effective amount may also be administered through continuous dosing, such as through infusion or with an implant.

### Compounds

The present invention is directed to a compound, a prodrug thereof, or a pharmaceutically acceptable salt thereof, represented by formula (I):

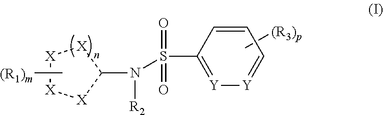

wherein:
dashed lines are independently single or double bonds;
R¹ is selected from the group consisting of:
   C₁-C₄ alkyl,
   halogen,
   C(O)-phenyl,
   C(O)—NR⁴R⁵,
   C(O)-pyrrolidine optionally substituted with one or more C₁-C₄ alkyl,
   tetrahydropyran optionally substituted with one or more C₁-C₄ alkyl,
   phenyl optionally substituted with one or more C₁-C₄ alkyl or —OR⁶,
   pyridine optionally substituted with one or more C₁-C₄ alkyl,
   thiazole optionally substituted with a group selected from the group consisting of:
      C₁-C₄ alkyl,
      C₃-C₆ cycloalkyl,
      R⁶—OH,
      R⁶—NR⁶R⁶,
      CN,
      C(O)—OH,
      C(O)—OR⁶,
      C(O)—NH₂,
      C(O)—NHR⁶,
      C(O)—NR⁶R⁶, and
      tetrazole optionally substituted with one or more C₁-C₄ alkyl,
   oxazole optionally substituted with one or more C₁-C₄ alkyl or C₃-C₇ cycloalkyl,
   pyrazole optionally substituted with one or more C₁-C₄ alkyl or C₃-C₇ cycloalkyl,
   imidazole optionally substituted with one or more C₁-C₄ alkyl or C₃-C₇ cycloalkyl, wherein the C₁-C₄ alkyl is optionally substituted with phenyl, and
   piperidine optionally substituted with one or more of C₁-C₄ alkyl or C(O)—R⁶,
or two R are joined together to form a 6-membered aryl ring, a 5-membered saturated heterocyclic ring, or a 5-membered unsaturated heterocyclic ring, wherein the aryl ring is optionally substituted with one or more of C₁-C₄ alkyl or one or more halogen, and wherein either heterocyclic ring optionally contains a second heteroatom selected from O and S, and either heterocyclic ring is optionally substituted with one or more groups selected from the group consisting of C₁-C₄ alkyl, phenyl, and oxo,
or two R are joined together to form a phenyl group that is optionally substituted with one or more of C₁-C₄ alkyl or halogen;
R² is hydrogen or C₁-C₄ alkyl;
R³ is selected from the group consisting of C₁-C₄ alkyl, C₁-C₄ alkoxy, C₁-C₄ trihaloalkyl, CN, and halogen,
or two R³ are joined together to form a 5- to 6-membered unsaturated heterocyclic ring that optionally contains a second heteroatom selected from N and S;
R⁴ and R⁵ are each independently selected from the group consisting of H, C₁-C₄ alkyl, 4- to 6-membered heterocyclic ring containing O, 4- to 6-membered cycloalkyl, and 4to 6-membered heteroaryl, wherein the C₁-C₄ alkyl is optionally substituted with a group selected from the group consisting of C₃-C₇ cycloalkyl, OH, OR⁶, phenyl, C(O)—NH₂, and C(O)—NR⁶R⁶,
or R⁴ and R⁵ together with the nitrogen atom to which they are joined form a 5- to 7-membered heterocyclic ring optionally containing a second heteroatom selected from N, O, and S, wherein the heterocyclic ring is optionally substituted with one or more groups independently selected from the group consisting of C₁-C₄ alkyl, C₃-C₇ cycloalkyl, C₁-C₄ alkoxy, halogen, hydroxy, NH₂, NHR⁶, NR⁶R⁶, cyano, C(O)—R⁶, C(O)NH₂, wherein the C₁-C₄ alkyl is optionally substituted with OH or OR⁶,
or R⁴ and R⁵ together with the nitrogen atom to which they are joined form a 7- to 9-membered heterobicyclic ring optionally containing one or two additional N atoms, and optionally substituted with C₁-C₄ alkyl;
R⁶ is C₁-C₄ alkyl;
X is selected from the group consisting of C, CH, CH₂, O, N, NR, and S, as allowed by valency;
Y is C, CH or N, as allowed by valency;
m is 1-2;
n is 1-2; and
p is 0-3.

In certain examples, a compound of formula (I), a prodrug thereof, or a pharmaceutically acceptable salt thereof, is provided:

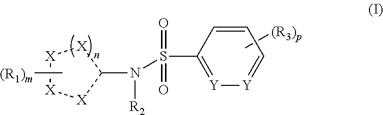

wherein:
dashed lines are independently single or double bonds;
R¹ is selected from the group consisting of:
   C₁ alkyl,
   F,
   C(O)-phenyl,
   C(O)—NR⁴R⁵,
   C(O)-pyrrolidine optionally substituted with C₁ alkyl,
   tetrahydropyran,
   phenyl optionally substituted with methoxy,
   pyridine,
   thiazole optionally substituted with a group selected from the group consisting of:
      C₁ alkyl,
      C₃ cycloalkyl,
      R⁶—OH,
      R⁶—NR⁶R⁶,
      CN,
      C(O)—OH,
      C(O)—OR⁶,
      C(O)—NH₂,
      C(O)—NHR⁶,
      C(O)—NR⁶R⁶, and
      tetrazole,
   oxazole optionally substituted with C₃ cycloalkyl,
   pyrazole optionally substituted with C₁ alkyl or C₃ cycloalkyl,
   imidazole optionally substituted with one or more of C₁ alkyl or C₃ cycloalkyl,
   wherein the C₁ alkyl is optionally substituted with phenyl, and
   piperidine optionally substituted with C(O)—R⁶,
or two R are joined together to form a 6-membered aryl ring, a 5-membered saturated heterocyclic ring, or a 5-membered unsaturated heterocyclic ring, wherein the aryl ring is optionally substituted with chloro, and wherein either heterocyclic ring optionally contains a second heteroatom selected from O and S, and either heterocyclic ring is optionally substituted with one or more groups selected from the group consisting of C₁-C₃ alkyl, phenyl, and oxo,
or two R are joined together to form a phenyl group that is optionally substituted with Cl;
R² is hydrogen;
R³ is selected from the group consisting of C₁ alkyl, C₁ alkoxy, trifluoromethyl, chloro, fluoro,
or two R³ are joined together to form a 5- to 6-membered unsaturated heterocyclic ring that optionally contains a second heteroatom selected from N and S;
R⁴ and R⁵ are each independently selected from the group consisting of H, C₁-C₃ alkyl, 4-membered heterocyclic ring containing O, 5- or 6-membered cycloalkyl, and 6-membered heteroaryl, wherein the C₁-C₃ alkyl is optionally substituted with a group selected from the group consisting of C₃-cycloalkyl, OH, OR⁶, phenyl, and C(O)—NH₂,
or R⁴ and R⁵ together with the nitrogen atom to which they are joined form a 5- to 7-membered heterocyclic ring optionally containing a second heteroatom selected from N, O, and S, wherein the heterocyclic ring is optionally substituted with one or more groups independently selected from the group consisting of C₁-C₃ alkyl, C₃ cycloalkyl, halogen, NH₂, NHR⁶, NR⁶R⁶, cyano, C(O)—C₁-C₃ alkyl, C(O)NH₂, wherein the C₁-C₃ alkyl is optionally substituted with OH,
or R⁴ and R⁵ together with the nitrogen atom to which they are joined form a 7- to 9-membered heterobicyclic ring optionally containing one or two additional N atoms, and optionally substituted with C₁ alkyl;
R⁶ is C₁ alkyl;
X is selected from the group consisting of C, CH, CH₂, O, N, NR, and S, as allowed by valency;
Y is C, CH or N, as allowed by valency;
m is 1-2;
n is 1-2; and
p is 0-3.

In certain examples of formula (I), the dashed lines are single bonds. In certain examples of formula (I), the dashed lines are independently single and double bonds.

In certain examples of formula (I), R¹ is C₁-C₄ alkyl. In certain examples of formula (I), R¹ is halogen. In certain examples of formula (I), R¹ is F. In certain examples of formula (I), R¹ is Cl. In certain examples of formula (I), R¹ is Br. In certain examples of formula (I), R¹ is C(O)-phenyl. In certain examples of formula (I), R¹ is C(O)—NR⁴R⁵. In certain examples of formula (I), R¹ is C(O)-pyrrolidine optionally substituted with one or more C₁-C₄ alkyl. In certain examples of formula (I), R¹ is tetrahydropyran optionally substituted with one or more C₁-C₄ alkyl. In certain examples of formula (I), R¹ is phenyl optionally substituted with one or more C₁-C₄ alkyl or —OR⁶. In certain examples of formula (I), R¹ is pyridine optionally substituted with one or more C₁-C₄ alkyl.

In certain examples of formula (I), R¹ is thiazole. In certain examples of formula (I), R¹ is thiazole substituted with C₁-C₄ alkyl. In certain examples of formula (I), R¹ is thiazole substituted with C₃-C₆ cycloalkyl. In certain examples of formula (I), R¹ is thiazole substituted with R⁶—OH. In certain examples of formula (I), R¹ is thiazole substituted with R⁶—NR⁶R⁶. In certain examples of formula (I), R¹ is thiazole substituted with CN. In certain examples of formula (I), R¹ is thiazole substituted with C(O)—OH. In certain examples of formula (I), R¹ is thiazole substituted with C(O)—OR⁶. In certain examples of formula (I), R¹ is thiazole substituted with C(O)—NH₂. In certain examples of formula (I), R¹ is thiazole substituted with C(O)—NHR⁶. In certain examples of formula (I), R¹ is thiazole substituted with C(O)—NR⁶R⁶. In certain examples of formula (I), R¹ is thiazole substituted with tetrazole optionally substituted with one or more C₁-C₄ alkyl.

In certain examples of formula (I), R¹ is oxazole optionally substituted with one or more C₁-C₄ alkyl or C₃-C₇ cycloalkyl. In certain examples of formula (I), R¹ is pyrazole optionally substituted with one or more C₁-C₄ alkyl or C₃-C₇ cycloalkyl. In certain examples of formula (I), R¹ is imidazole optionally substituted with one or more C₁-C₄ alkyl or C₃-C₇ cycloalkyl, wherein the C₁-C₄ alkyl is optionally substituted with phenyl. In certain examples of formula (I), R¹ is piperidine optionally substituted with one or more of C₁-C₄ alkyl or C(O)—R⁶.

In certain examples of formula (I), two R are joined together to form a 6-membered aryl ring. In certain examples of formula (I), two R are joined together to form a 5-membered saturated heterocyclic ring. In certain examples of formula (I), two R¹ are joined together to form a 5-membered unsaturated heterocyclic ring. In certain examples of formula (I), the aryl ring is optionally substituted with one or more of C₁-C₄ alkyl or one or more halogen. In certain examples of formula (I), the heterocyclic ring optionally contains a second heteroatom selected from O and S. In certain examples of formula (I), the heterocyclic ring is optionally substituted with one or more groups selected from the group consisting of C₁-C₄ alkyl, phenyl, and oxo.

In certain examples of formula (I), two R are joined together to form a phenyl group that is optionally substituted with one or more of C₁-C₄ alkyl or halogen.

In certain examples of formula (I), R² is hydrogen. In certain examples of formula (I), R² is C₁-C₄ alkyl.

In certain examples of formula (I), R³ is C₁-C₄ alkyl. In certain examples of formula (I), R³ is C₁-C₄ alkoxy. In certain examples of formula (I), R³ is C₁-C₄ trihaloalkyl. In certain examples of formula C₁ trihaloalkyl is trichloromethyl. In certain examples of formula C₁ trihaloalkyl is trifluoromethyl. In certain examples of formula (I), R³ is CN. In certain examples of formula (I), R³ is halogen. In certain examples of formula (I), halogen is F. In certain examples of formula (I), halogen is Cl. In certain examples of formula (I), halogen is Br.

In certain examples of formula (I), two R³ are joined together to form a 5-membered unsaturated heterocyclic ring that optionally contains a second heteroatom selected from N and S. In certain examples of formula (I), two R³ are joined together to form a 6-membered unsaturated heterocyclic ring that optionally contains a second heteroatom selected from N and S.

In certain examples of formula (I), R⁴ and R⁵ are each independently selected from H. In certain examples of formula (I), R⁴ and R⁵ are each independently selected from C₁-C₄ alkyl. In certain examples of formula (I), R⁴ and R⁵ are each independently selected from 4- to 6-membered heterocyclic ring containing O. In certain examples of formula (I), R⁴ and R⁵ are each independently selected from 4- to 6-membered cycloalkyl. In certain examples of formula (I), R⁴ and R⁵ are each independently selected from 4- to 6-membered heteroaryl. In certain examples of formula (I), C₁-C₄ alkyl is optionally substituted with C₃-C₇ cycloalkyl. In certain examples of formula (I), C₁-C₄ alkyl is optionally substituted with OH. In certain examples of formula (I), C₁-C₄ alkyl is optionally substituted with OR⁶. In certain examples of formula (I), C₁-C₄ alkyl is optionally substituted with phenyl In certain examples of formula (I), C₁-C₄ alkyl is optionally substituted with C(O)—NH₂. In certain examples of formula (I), C₁-C₄ alkyl is optionally substituted with C(O)—NR⁶R⁶.

In certain examples of formula (I), R⁴ and R⁵ together with the nitrogen atom to which they are joined form a 5-membered heterocyclic ring optionally containing a second heteroatom selected from N, O, and S. In certain examples of formula (I), R⁴ and R⁵ together with the nitrogen atom to which they are joined form a 6-membered heterocyclic ring optionally containing a second heteroatom selected from N, O, and S. In certain examples of formula (I), R⁴ and R⁵ together with the nitrogen atom to which they are joined form a 6-membered heterocyclic ring optionally containing a second heteroatom selected from N, O, and S.

In certain examples of formula (I), the heterocyclic ring is optionally substituted with C₁-C₄ alkyl. In certain examples of formula (I), the heterocyclic ring is optionally substituted with C₃-C₇ cycloalkyl. In certain examples of formula (I), the heterocyclic ring is optionally substituted with C₁-C₄ alkoxy. In certain examples of formula (I), the heterocyclic ring is optionally substituted with halogen. In certain examples of formula (I), the heterocyclic ring is optionally substituted with hydroxy. In certain examples of formula (I), the heterocyclic ring is optionally substituted with NH₂. In certain examples of formula (I), the heterocyclic ring is optionally substituted with NHR⁶. In certain examples of formula (I), the heterocyclic ring is optionally substituted with NR⁶R⁶. In certain examples of formula (I), the heterocyclic ring is optionally substituted with cyano. In certain examples of formula (I), the heterocyclic ring is optionally substituted with C(O)—R⁶. In certain examples of formula (I), the heterocyclic ring is optionally substituted with C(O)NH₂. In certain examples of formula (I), the C₁-C₄ alkyl is optionally substituted with OH. In certain examples of formula (I), the C₁-C₄ alkyl is optionally substituted with OR⁶.

In certain examples of formula (I), R⁴ and R⁵ together with the nitrogen atom to which they are joined form a 7-membered heterobicyclic ring optionally containing one or two additional N atoms, and optionally substituted with C₁-C₄ alkyl. In certain examples of formula (I), R⁴ and R⁵ together with the nitrogen atom to which they are joined form a 8-membered heterobicyclic ring optionally containing one or two additional N atoms, and optionally substituted with C₁-C₄ alkyl. In certain examples of formula (I), R⁴ and R⁵ together with the nitrogen atom to which they are joined form a 9-membered heterobicyclic ring optionally containing one or two additional N atoms, and optionally substituted with C₁-C₄ alkyl.

In certain examples of formula (I), R⁶ is C₁-C₄ alkyl.

In certain examples of formula (I), X is C, as allowed by valency. In certain examples of formula (I), X is CH, as allowed by valency. In certain examples of formula (I), X is CH₂, as allowed by valency. In certain examples of formula (I), X is O, as allowed by valency. In certain examples of formula (I), X is N, as allowed by valency. In certain examples of formula (I), X is NR, as allowed by valency. In certain examples of formula (I), X is S, as allowed by valency.

In certain examples of formula (I), Y is C, as allowed by valency. In certain examples of formula (I), Y is CH, as allowed by valency. In certain examples of formula (I), Y is N, as allowed by valency.

In certain examples of formula (I), m is 1. In certain examples of formula (I), m is 2.

In certain examples of formula (I), n is 1. In certain examples of formula (I), n is 2.

In certain examples of formula (I), p is 0. In certain examples of formula (I), p is 1. In certain examples of formula (I), p is 2. In certain examples of formula (I), p is 3.

In certain examples of formula (I), the group

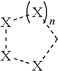

is selected from the group consisting of:

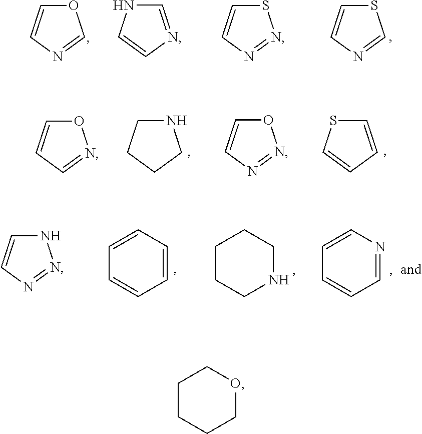

In certain examples of formula (I), the group:

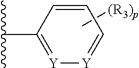

is selected from the group consisting of:

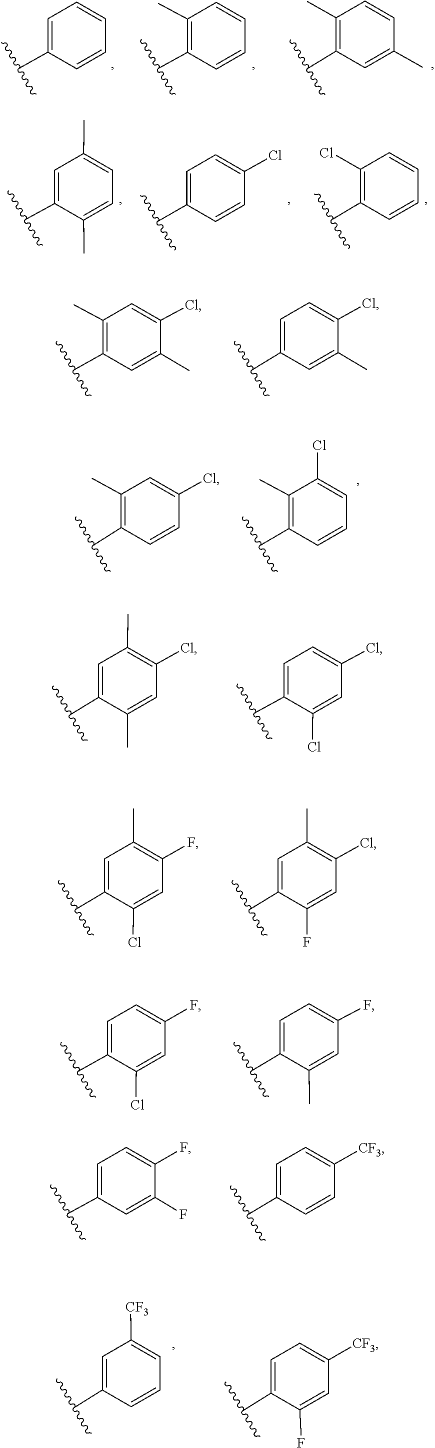

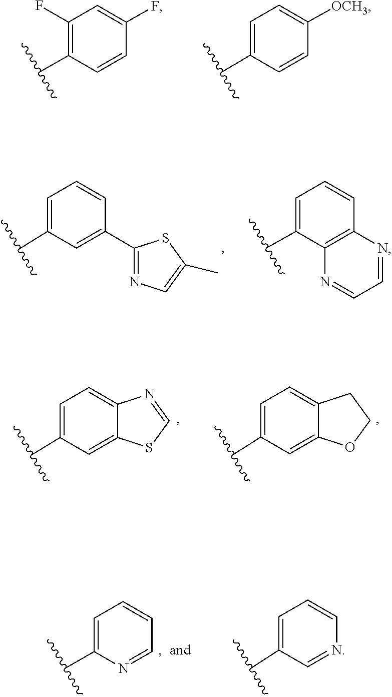

Exemplary Compounds of the present invention

| Example | | |
|---|---|---|
| No. | Structure | Chemical Name |
| 1 | 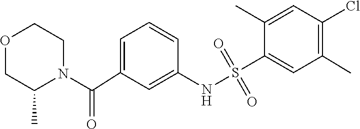 [SMILES: Cc1cc(S(=O)(=O)Nc2cccc(C(=O)N3CCOC[C@H]3C)c2)c(C)cc1Cl] | (R)-4-chloro-2,5-dimethyl-N-(3-(3- methylmorpholine-4- carbonyl)phenyl)benzenesulfonamide |
| 2 | 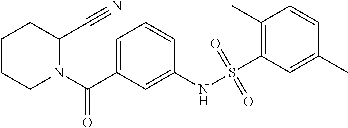 [SMILES: Cc1ccc(C)c(S(=O)(=O)Nc2cccc(C(=O)N3CCCCC3C#N)c2)c1] | N-(3-(2-cyanopiperidine-1- carbonyl)phenyl)-2,5- dimethylbenzenesulfonamide |
| 3 | 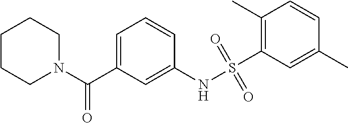 [SMILES: Cc1ccc(C)c(S(=O)(=O)Nc2cccc(C(=O)N3CCCCC3)c2)c1] | 2-methyl-N-(3-(piperidine-1- carbonyl)phenyl)benzenesulfonamide |
| 4 | 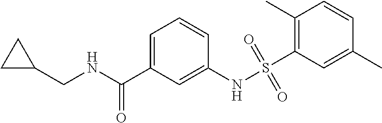 [SMILES: Cc1ccc(C)c(S(=O)(=O)Nc2cccc(C(=O)NCC3CC3)c2)c1] | N-(cyclopropylmethyl)-3-((2,5- dimethylphenyl)sulfonamido)benzamide |
| 5 | 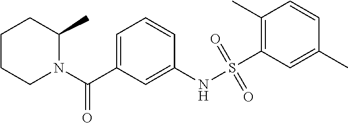 [SMILES: Cc1ccc(C)c(S(=O)(=O)Nc2cccc(C(=O)N3CCCC[C@H]3C)c2)c1] | (R)-2,5-dimethyl-N-(3-(2- methylpiperidine-1- carbonyl)phenyl)benzenesulfonamide |
| 6 | 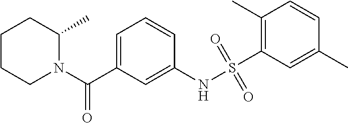 [SMILES: Cc1ccc(C)c(S(=O)(=O)Nc2cccc(C(=O)N3CCCC[C@@H]3C)c2)c1] | (S)-2,5-dimethyl-N-(3-(2- methylpiperidine-1- carbonyl)phenyl)benzenesulfonamide |
| 7 | 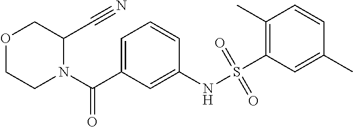 [SMILES: Cc1ccc(C)c(S(=O)(=O)Nc2cccc(C(=O)N3CCOCC3C#N)c2)c1] | N-(3-(3-cyanomorpholine-4- carbonyl)phenyl)-2,5- dimethylbenzenesulfonamide |
| 8 | 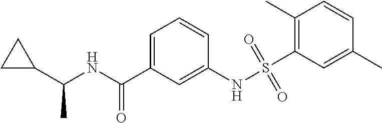 [SMILES: Cc1ccc(C)c(S(=O)(=O)Nc2cccc(C(=O)N[C@@H](C)C3CC3)c2)c1] | (S)-N-(1-cyclopropylethyl)-3-((2,5- dimethylphenyl)sulfonamido)benzamide |
| 9 | 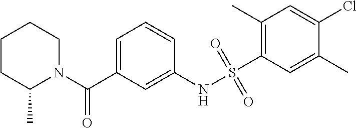 [SMILES: Cc1cc(S(=O)(=O)Nc2cccc(C(=O)N3CCCC[C@H]3C)c2)c(C)cc1Cl] | (R)-4-chloro-2,5-dimethyl-N-(3-(2- methylpiperidine-1- carbonyl)phenyl)benzenesulfonamide |
| 10 | 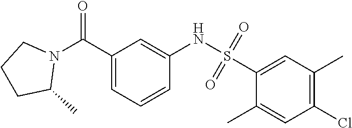 [SMILES: Cc1cc(S(=O)(=O)Nc2cccc(C(=O)N3CCC[C@H]3C)c2)c(C)cc1Cl] | (R)-4-chloro-2,5-dimethyl-N-(3-(2- methylpyrrolidine-1- carbonyl)phenyl)benzenesulfonamide |
| 11 | 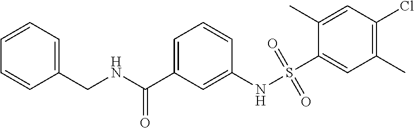 [SMILES: Cc1cc(S(=O)(=O)Nc2cccc(C(=O)NCc3ccccc3)c2)c(C)cc1Cl] | N-benzyl-3-((4-chloro-2,5- dimethylphenyl)sulfonamido)benzamide |
| 12 | 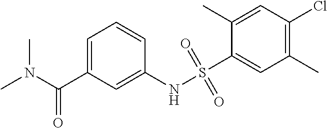 [SMILES: Cc1cc(S(=O)(=O)Nc2cccc(C(=O)N(C)C)c2)c(C)cc1Cl] | 3-((4-chloro-2,5- dimethylphenyl)sulfonamido)-N,N- dimethylbenzamide |
| 13 | 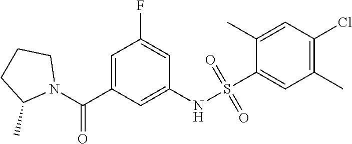 [SMILES: Cc1cc(S(=O)(=O)Nc2cc(F)cc(C(=O)N3CCC[C@H]3C)c2)c(C)cc1Cl] | (R)-4-chloro-N-(3-fluoro-5-(2- methylpyrrolidine-1-carbonyl)phenyl)- 2,5-dimethylbenzenesulfonamide |
| 14 | 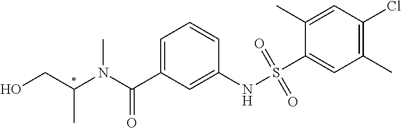 [SMILES: Cc1cc(S(=O)(=O)Nc2cccc(C(=O)N(C)C(C)CO)c2)c(C)cc1Cl] | 3-((4-chloro-2,5- dimethylphenyl)sulfonamido)-N-(1- hydroxypropan-2-yl)-N- methylbenzamide |
| 15 | 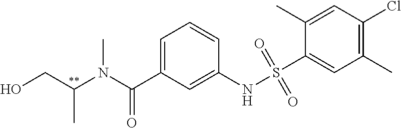 [SMILES: Cc1cc(S(=O)(=O)Nc2cccc(C(=O)N(C)C(C)CO)c2)c(C)cc1Cl] | 3-((4-chloro-2,5- dimethylphenyl)sulfonamido)-N-(1- hydroxypropan-2-yl)-N- methylbenzamide |
| 16 | 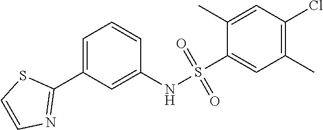 [SMILES: Cc1cc(S(=O)(=O)Nc2cccc(-c3nccs3)c2)c(C)cc1Cl] | 4-chloro-2,5-dimethyl-N-(3-(thiazol-2- yl)phenyl)benzenesulfonamide |
| 17 | 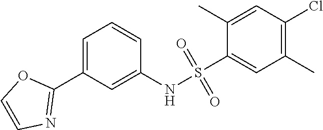 [SMILES: Cc1cc(S(=O)(=O)Nc2cccc(-c3ncco3)c2)c(C)cc1Cl] | 4-chloro-2,5-dimethyl-N-(3-(oxazol-2- yl)phenyl)benzenesulfonamide |
| 18 | 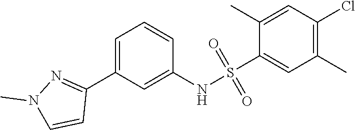 [SMILES: Cc1cc(S(=O)(=O)Nc2cccc(-c3ccn(C)n3)c2)c(C)cc1Cl] | 4-chloro-2,5-dimethyl-N-(3-(1-methyl- 1H-pyrazol-3- yl)phenyl)benzenesulfonamide |
| 19 | 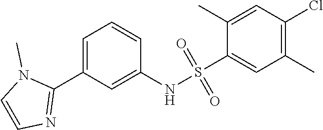 [SMILES: Cc1cc(S(=O)(=O)Nc2cccc(-c3nccn3C)c2)c(C)cc1Cl] | 4-chloro-2,5-dimethyl-N-(3-(1-methyl- 1H-imidazol-2- yl)phenyl)benzenesulfonamide |
| 20 | 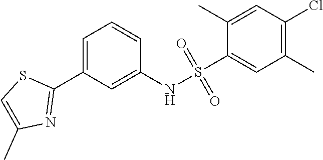 [SMILES: Cc1csc(-c2cccc(NS(=O)(=O)c3cc(C)c(Cl)cc3C)c2)n1] | 4-chloro-2,5-dimethyl-N-(3-(4- methylthiazol-2- yl)phenyl)benzenesulfonamide |
| 21 | 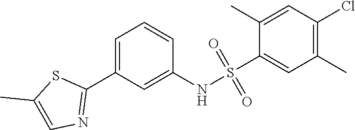 [SMILES: Cc1cnc(-c2cccc(NS(=O)(=O)c3cc(C)c(Cl)cc3C)c2)s1] | 4-chloro-2,5-dimethyl-N-(3-(5- methylthiazol-2- yl)phenyl)benzenesulfonamide |
| 22 | 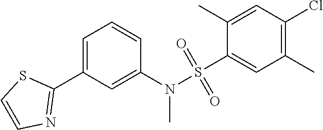 [SMILES: Cc1cc(S(=O)(=O)N(C)c2cccc(-c3nccs3)c2)c(C)cc1Cl] | 4-chloro-N,2,5-trimethyl-N-(3-(thiazol- 2-yl)phenyl)benzenesulfonamide |
| 23 | 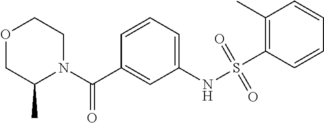 [SMILES: Cc1ccccc1S(=O)(=O)Nc1cccc(C(=O)N2CCOC[C@@H]2C)c1] | (S)-2-methyl-N-(3-(3- methylmorpholine-4- carbonyl)phenyl)benzenesulfonamide |
| 24 | 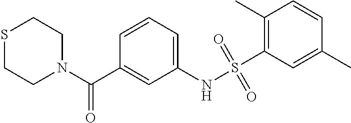 [SMILES: Cc1ccc(C)c(S(=O)(=O)Nc2cccc(C(=O)N3CCSCC3)c2)c1] | 2,5-dimethyl-N-(3-(thiomorpholine-4- carbonyl)phenyl)benzenesulfonamide |
| 25 | 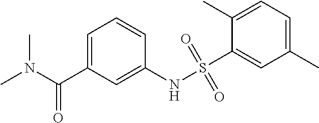 | 3-((2,5-dimethylphenyl)sulfonamido)- N,N-dimethylbenzamide |
| 26 | 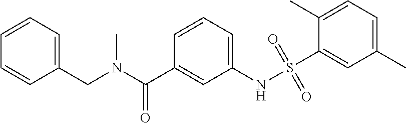 [SMILES: Cc1ccc(C)c(S(=O)(=O)Nc2cccc(C(=O)N(C)Cc3ccccc3)c2)c1] | N-benzyl-3-((2,5- dimethylphenyl)sulfonamido)-N- methylbenzamide |
| 27 | 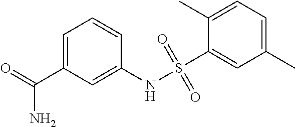 [SMILES: Cc1ccc(C)c(S(=O)(=O)Nc2cccc(C(N)=O)c2)c1] | 3-((2,5- dimethylphenyl)sulfonamido)benzamide |
| 28 | 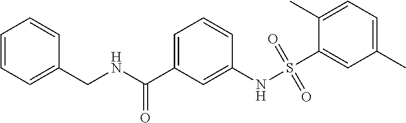 [SMILES: Cc1ccc(C)c(S(=O)(=O)Nc2cccc(C(=O)NCc3ccccc3)c2)c1] | N-benzyl-3-((2,5- dimethylphenyl)sulfonamido)benzamide |
| 29 | 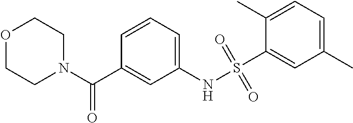 [SMILES: Cc1ccc(C)c(S(=O)(=O)Nc2cccc(C(=O)N3CCOCC3)c2)c1] | 2,5-dimethyl-N-(3-(morpholine-4- carbonyl)phenyl)benzenesulfonamide |
| 30 | 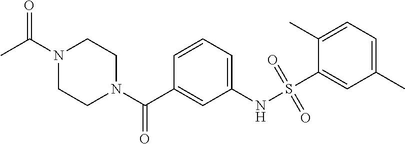 [SMILES: CC(=O)N1CCN(C(=O)c2cccc(NS(=O)(=O)c3cc(C)ccc3C)c2)CC1] | N-(3-(4-acetylpiperazine-1- carbonyl)phenyl)-2,5- dimethylbenzenesulfonamide |
| 31 | 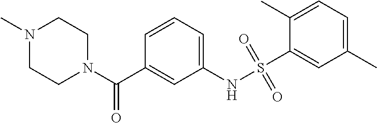 | 2,5-dimethyl-N-(3-(4-methylpiperazine- 1-carbonyl)phenyl)benzenesulfonamide |
| 32 | 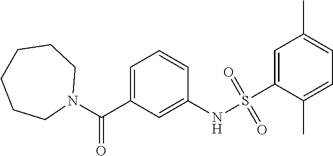 [SMILES: Cc1ccc(C)c(S(=O)(=O)Nc2cccc(C(=O)N3CCCCCC3)c2)c1] | N-(3-(azepane-1-carbonyl)phenyl)-2,5- dimethylbenzenesulfonamide |
| 33 | 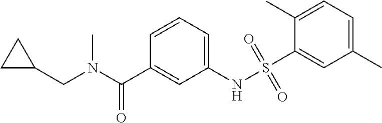 [SMILES: Cc1ccc(C)c(S(=O)(=O)Nc2cccc(C(=O)N(C)CC3CC3)c2)c1] | N-(cyclopropylmethyl)-3-((2,5- dimethylphenyl)sulfonamido)-N- methylbenzamide |
| 34 | 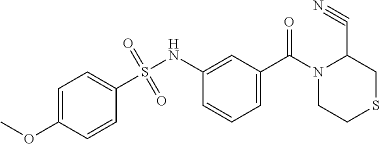 [SMILES: COc1ccc(S(=O)(=O)Nc2cccc(C(=O)N3CCSCC3C#N)c2)cc1] | N-(3-(3-cyanothiomorpholine-4- carbonyl)phenyl)-4- methoxybenzenesulfonamide |
| 35 | 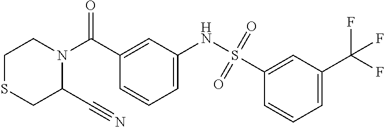 [SMILES: N#CC1CSCCN1C(=O)c1cccc(NS(=O)(=O)c2cccc(C(F)(F)F)c2)c1] | N-(3-(3-cyanothiomorpholine-4- carbonyl)phenyl)-3- (trifluoromethyl)benzenesulfonamide |
| 36 | 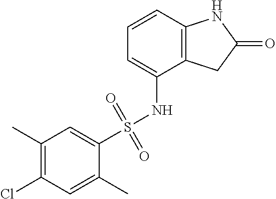 [SMILES: Cc1cc(S(=O)(=O)Nc2cccc3c2CC(=O)N3)c(C)cc1Cl] | 4-chloro-2,5-dimethyl-N-(2-oxoindolin- 4-yl)benzenesulfonamide |
| 37 | 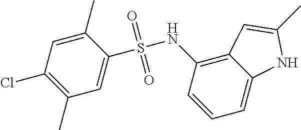 [SMILES: Cc1cc2c(NS(=O)(=O)c3cc(C)c(Cl)cc3C)cccc2[nH]1] | 4-chloro-2,5-dimethyl-N-(2-methyl-1H- indol-4-yl)benzenesulfonamide |
| 38 | 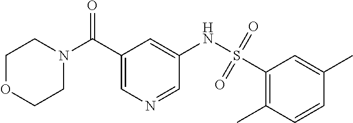 [SMILES: Cc1ccc(C)c(S(=O)(=O)Nc2cncc(C(=O)N3CCOCC3)c2)c1] | 2,5-dimethyl-N-(5-(morpholine-4- carbonyl)pyridin-3- yl)benzenesulfonamide |
| 39 | 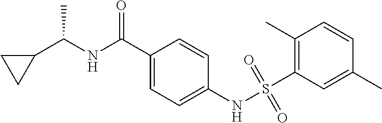 [SMILES: Cc1ccc(C)c(S(=O)(=O)Nc2ccc(C(=O)N[C@@H](C)C3CC3)cc2)c1] | (S)-N-(1-cyclopropylethyl)-4-((2,5- dimethylphenyl)sulfonamido)benzamide |
| 40 | 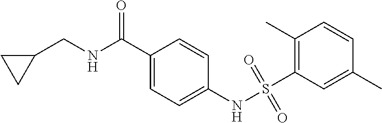 [SMILES: Cc1ccc(C)c(S(=O)(=O)Nc2ccc(C(=O)NCC3CC3)cc2)c1] | N-(cyclopropylmethyl)-4-((2,5- dimethylphenyl)sulfonamido)benzamide |
| 41 | 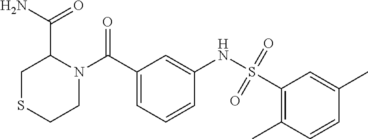 [SMILES: Cc1ccc(C)c(S(=O)(=O)Nc2cccc(C(=O)N3CCSCC3C(N)=O)c2)c1] | 4-(3-((2,5- dimethylphenyl)sulfonamido)benzoyl) thiomorpholine-3-carboxamide |
| 42 | 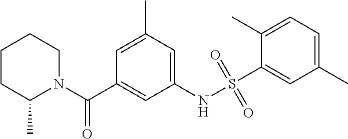 [SMILES: Cc1cc(NS(=O)(=O)c2cc(C)ccc2C)cc(C(=O)N2CCCC[C@H]2C)c1] | (R)-2,5-dimethyl-N-(3-methyl-5-(2- methylpiperidine-1- carbonyl)phenyl)benzenesulfonamide |
| 43 | 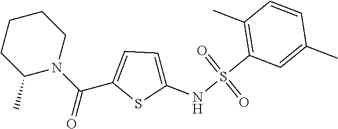 [SMILES: Cc1ccc(C)c(S(=O)(=O)Nc2ccc(C(=O)N3CCCC[C@H]3C)s2)c1] | (R)-2,5-dimethyl-N-(5-(2- methylpiperidine-1-carbonyl)thiopheN- 2-yl)benzenesulfonamide |
| 44 | 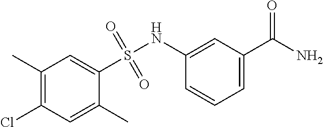 [SMILES: Cc1cc(S(=O)(=O)Nc2cccc(C(N)=O)c2)c(C)cc1Cl] | 3-((4-chloro-2,5- dimethylphenyl)sulfonamido)benzamide |
| 45 | 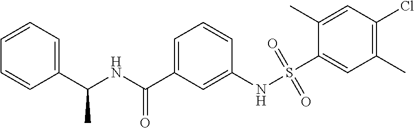 [SMILES: Cc1cc(S(=O)(=O)Nc2cccc(C(=O)N[C@@H](C)c3ccccc3)c2)c(C)cc1Cl] | (S)-3-((4-chloro-2,5- dimethylphenyl)sulfonamido)-N-(1- phenylethyl)benzamide |
| 46 | 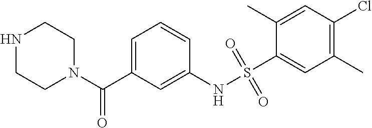 [SMILES: Cc1cc(S(=O)(=O)Nc2cccc(C(=O)N3CCNCC3)c2)c(C)cc1Cl] | 4-chloro-2,5-dimethyl-N-(3-(piperazine- 1-carbonyl)phenyl)benzenesulfonamide |
| 47 | 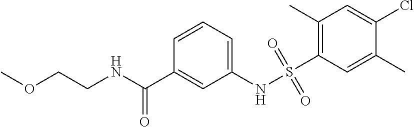 [SMILES: COCCNC(=O)c1cccc(NS(=O)(=O)c2cc(C)c(Cl)cc2C)c1] | 3-((4-chloro-2,5- dimethylphenyl)sulfonamido)-N-(2- methoxyethyl)benzamide |
| 48 | 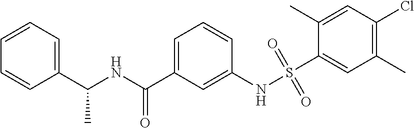 [SMILES: Cc1cc(S(=O)(=O)Nc2cccc(C(=O)N[C@H](C)c3ccccc3)c2)c(C)cc1Cl] | (R)-3-((4-chloro-2,5- dimethylphenyl)sulfonamido)-N-(1- phenylethyl)benzamide |
| 49 | 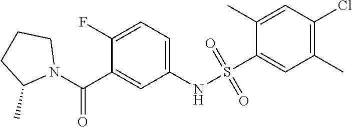 [SMILES: Cc1cc(S(=O)(=O)Nc2ccc(F)c(C(=O)N3CCC[C@H]3C)c2)c(C)cc1Cl] | (R)-4-chloro-N-(4-fluoro-3-(2- methylpyrrolidine-1-carbonyl)phenyl)- 2,5-dimethylbenzenesulfonamide |
| 50 | 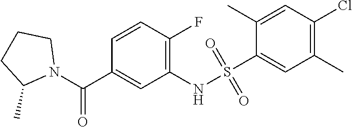 [SMILES: Cc1cc(S(=O)(=O)Nc2cc(C(=O)N3CCC[C@H]3C)ccc2F)c(C)cc1Cl] | (R)-4-chloro-N-(2-fluoro-5-(2- methylpyrrolidine-1-carbonyl)phenyl)- 2,5-dimethylbenzenesulfonamide |
| 51 | 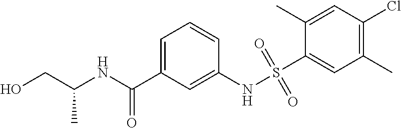 [SMILES: Cc1cc(S(=O)(=O)Nc2cccc(C(=O)N[C@H](C)CO)c2)c(C)cc1Cl] | (R)-3-((4-chloro-2,5- dimethylphenyl)sulfonamido)-N-(1- hydroxypropan-2-yl)benzamide |
| 52 | 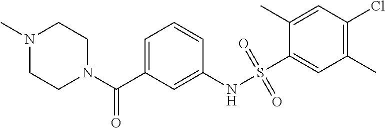 [SMILES: Cc1cc(S(=O)(=O)Nc2cccc(C(=O)N3CCN(C)CC3)c2)c(C)cc1Cl] | 4-chloro-2,5-dimethyl-N-(3-(4- methylpiperazine-1- carbonyl)phenyl)benzenesulfonamide |
| 53 | 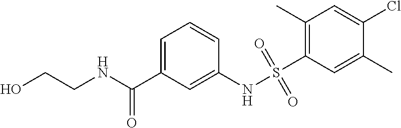 [SMILES: Cc1cc(S(=O)(=O)Nc2cccc(C(=O)NCCO)c2)c(C)cc1Cl] | 3-((4-chloro-2,5- dimethylphenyl)sulfonamido)-N-(2- hydroxyethyl)benzamide |
| 54 | 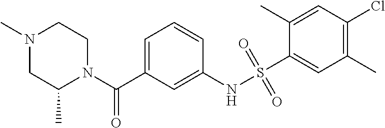 [SMILES: Cc1cc(S(=O)(=O)Nc2cccc(C(=O)N3CCN(C)C[C@H]3C)c2)c(C)cc1Cl] | (R)-4-chloro-N-(3-(2,4- dimethylpiperazine-1-carbonyl)phenyl)- 2,5-dimethylbenzenesulfonamide |
| 55 | 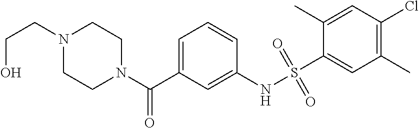 [SMILES: Cc1cc(S(=O)(=O)Nc2cccc(C(=O)N3CCN(CCO)CC3)c2)c(C)cc1Cl] | 4-chloro-N-(3-(4-(2- hydroxyethyl)piperazine-1- carbonyl)phenyl)-2,5- dimethylbenzenesulfonamide |
| 56 | 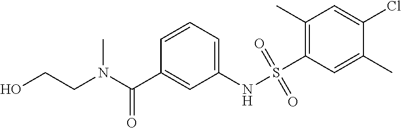 [SMILES: Cc1cc(S(=O)(=O)Nc2cccc(C(=O)N(C)CCO)c2)c(C)cc1Cl] | 3-((4-chloro-2,5- dimethylphenyl)sulfonamido)-N-(2- hydoxyethyl)-N-methylbenzamide |
| 57 | 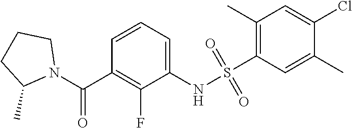 [SMILES: Cc1cc(S(=O)(=O)Nc2cccc(C(=O)N3CCC[C@H]3C)c2F)c(C)cc1Cl] | (R)-4-chloro-N-(2-fluoro-3-(2- methylpyrrolidine-1-carbonyl)phenyl)- 2,5-dimethylbenzenesulfonamide |
| 58 | 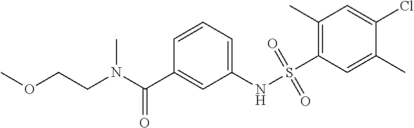 [SMILES: COCCN(C)C(=O)c1cccc(NS(=O)(=O)c2cc(C)c(Cl)cc2C)c1] | 3-((4-chloro-2,5- dimethylphenyl)sulfonamido)-N-(2- methoxyethyl)-N-methylbenzamide |
| 59 | 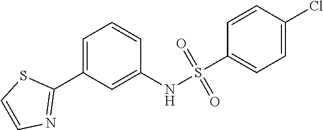 [SMILES: O=S(=O)(Nc1cccc(-c2nccs2)c1)c1ccc(Cl)cc1] | 4-chloro-N-(3-(thiazol-2- yl)phenyl)benzenesulfonamide |
| 60 | 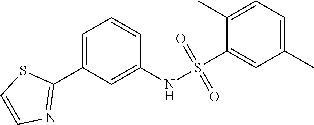 [SMILES: Cc1ccc(C)c(S(=O)(=O)Nc2cccc(-c3nccs3)c2)c1] | 2,5-dimethyl-N-(3-thiazol-2- yl)phenyl)benzenesulfonamide |
| 61 | 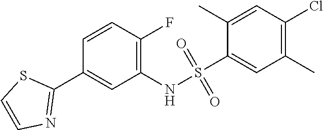 [SMILES: Cc1cc(S(=O)(=O)Nc2cc(-c3nccs3)ccc2F)c(C)cc1Cl] | 4-chloro-N-(2-fluoro-5-(thiazol-2- yl)phenyl)-2,5- dimethylbenzenesulfonamide |
| 62 | 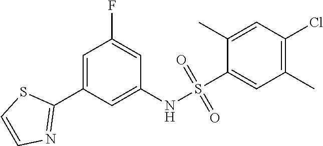 [SMILES: Cc1cc(S(=O)(=O)Nc2cc(F)cc(-c3nccs3)c2)c(C)cc1Cl] | 4-chloro-N-(3-fluoro-5-(thiazol-2- yl)phenyl)-2,5- dimethylbenzenesulfonamide |
| 63 | 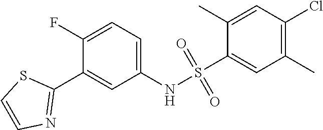 [SMILES: Cc1cc(S(=O)(=O)Nc2ccc(F)c(-c3nccs3)c2)c(C)cc1Cl] | 4-chloro-N-(4-fluoro-3-(thiazol-2- yl)phenyl)-2,5- dimethylbenzenesulfonamide |
| 64 | 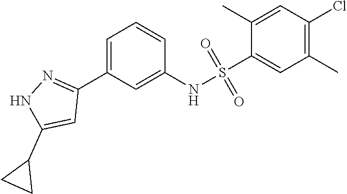 [SMILES: Cc1cc(S(=O)(=O)Nc2cccc(-c3cc(C4CC4)[nH]n3)c2)c(C)cc1Cl] | 4-chloro-N-(3-(5-cyclopropyl-1H- pyrazol-3-yl)phenyl)-2,5- dimethylbenzenesulfonamide |
| 65 | 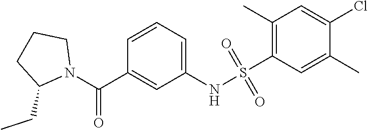 [SMILES: CC[C@@H]1CCCN1C(=O)c1cccc(NS(=O)(=O)c2cc(C)c(Cl)cc2C)c1] | (R)-4-chloro-N-(3-(2- ethylpyrrolidine-1- carbonyl)phenyl)-2,5- dimethylbenzenesulfonamide |
| 66 | 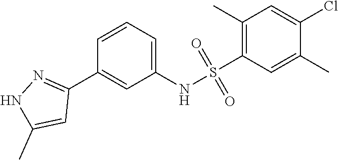 [SMILES: Cc1cc(-c2cccc(NS(=O)(=O)c3cc(C)c(Cl)cc3C)c2)n[nH]1] | 4-chloro-2,5-dimethyl-N-(3-(5-methyl- 1H-pyrazol-3- yl)phenyl)benzenesulfonamide |
| 67 | 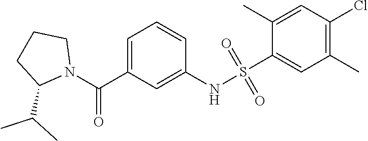 [SMILES: Cc1cc(S(=O)(=O)Nc2cccc(C(=O)N3CCC[C@H]3C(C)C)c2)c(C)cc1Cl] | (S)-4-chloro-N-(3-(2- isopropylpyrrolidine-1- carbonyl)phenyl)-2,5- dimethylbenzenesulfonamide |
| 68 | 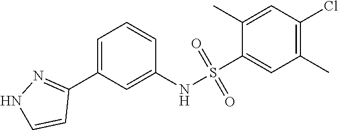 [SMILES: Cc1cc(S(=O)(=O)Nc2cccc(-c3cc[nH]n3)c2)c(C)cc1Cl] | N-(3-(1H-pyrazol-3-yl)phenyl)-4- chloro-2,5-dimethylbenzenesulfonamide |
| 69 | 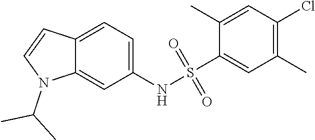 [SMILES: Cc1cc(S(=O)(=O)Nc2ccc3ccn(C(C)C)c3c2)c(C)cc1Cl] | 4-chloro-N-(1-isopropyl-1H-indol-6-yl)- 2,5-dimethylbenzenesulfonamide |
| 70 | 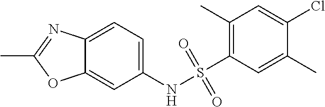 | 4-chloro-2,5-dimethyl-N-(2- methylbenzo[d]oxazol-6- yl)benzenesulfonamide |
| 71 | 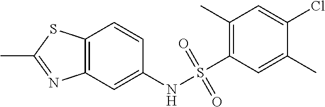 | 4-chloro-2,5-dimethyl-N-(2- methylbenzo[d]thiazol-5- yl)benzenesulfonamide |
| 72 | 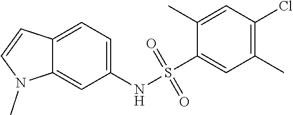 [SMILES: Cc1cc(S(=O)(=O)Nc2ccc3ccn(C)c3c2)c(C)cc1Cl] | 4-chloro-2,5-dimethyl-N-(1-methyl-1H- indol-6-yl)benzenesulfonamide |
| 73 | 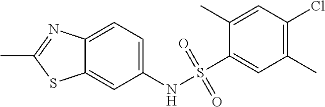 | 4-chloro-2,5-dimethyl-N-(2- methylbenzo[d]thiazol-6- yl)benzenesulfonamide |
| 74 | 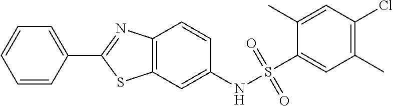 [SMILES: Cc1cc(S(=O)(=O)Nc2ccc3nc(-c4ccccc4)sc3c2)c(C)cc1Cl] | 4-chloro-2,5-dimethyl-N-(2- phenylbenzo[d]thiazol-6- yl)benzenesulfonamide |
| 75 | 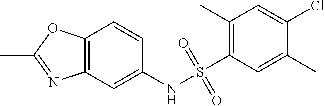 | 4-chloro-2,5-dimethyl-N-(2- methylbenzo[d]oxazol-5- yl)benzenesulfonamide |
| 76 | 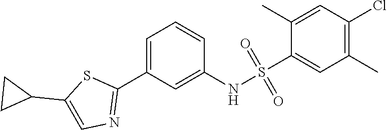 [SMILES: Cc1cc(S(=O)(=O)Nc2cccc(-c3ncc(C4CC4)s3)c2)c(C)cc1Cl] | 4-chloro-N-(3-(5-cyclopropylthiazol-2- yl)phenyl)-2,5- dimethylbenzenesulfonamide |
| 77 | 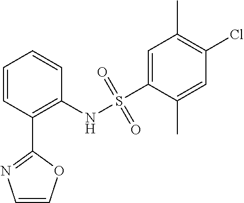 [SMILES: Cc1cc(S(=O)(=O)Nc2ccccc2-c2ncco2)c(C)cc1Cl] | 4-chloro-2,5-dimethyl-N-(2-(oxazol-2- yl)phenyl)benzenesulfonamide |
| 78 | 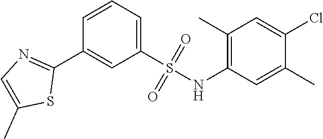 [SMILES: Cc1cnc(-c2cccc(S(=O)(=O)Nc3cc(C)c(Cl)cc3C)c2)s1] | N-(4-chloro-2,5-dimethylphenyl)-3-(5- methylthiazol-2-yl)benzenesulfonamide |
| 79 | 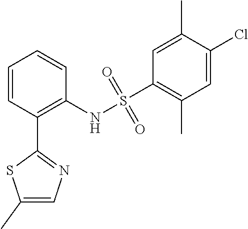 [SMILES: Cc1cnc(-c2ccccc2NS(=O)(=O)c2cc(C)c(Cl)cc2C)s1] | 4-chloro-2,5-dimethyl-N-(2-(5- methylthiazol-2- yl)phenyl)benzenesulfonamide |
| 80 | 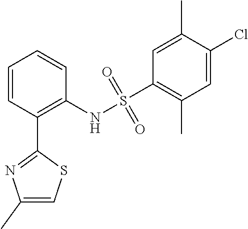 [SMILES: Cc1csc(-c2ccccc2NS(=O)(=O)c2cc(C)c(Cl)cc2C)n1] | 4-chloro-2,5-dimethyl-N-(2-(4- methylthiazol-2- yl)phenyl)benzenesulfonamide |
| 81 | 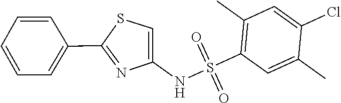 [SMILES: Cc1cc(S(=O)(=O)Nc2csc(-c3ccccc3)n2)c(C)cc1Cl] | 4-chloro-2,5-dimethyl-N-(2- phenylthiazol-4-yl)benzenesulfonamide |
| 82 | 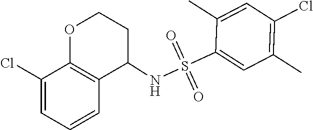 [SMILES: Cc1cc(S(=O)(=O)NC2CCOc3c(Cl)cccc32)c(C)cc1Cl] | 4-chloro-N-(8-chlorochroman-4-yl)-2,5- dimethylbenzenesulfonamide |
| 83 | 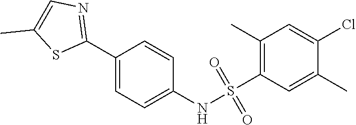 | 4-chloro-2,5-dimethyl-N-(4-(5- methylthiazol-2- yl)phenyl)benzenesulfonamide |
| 84 | 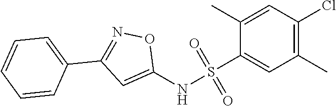 [SMILES: Cc1cc(S(=O)(=O)Nc2cc(-c3ccccc3)no2)c(C)cc1Cl] | 4-chloro-2,5-dimethyl-N-(3- phenylisoxazol-5- yl)benzenesulfonamide |
| 85 | 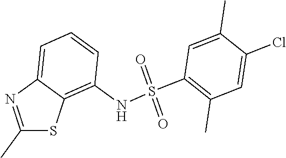 [SMILES: Cc1nc2cccc(NS(=O)(=O)c3cc(C)c(Cl)cc3C)c2s1] | 4-chloro-2,5-dimethyl-N-(2- methylbenzo[d]thiazol-7- yl)benzenesulfonamide |
| 86 | 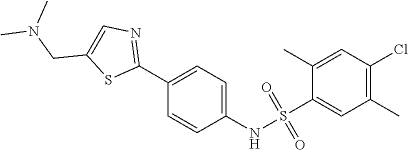 | 4-chloro-N-(4-(5- ((dimethylamino)methyl)thiazol-2- yl)phenyl)-2,5- dimethylbenzenesulfonamide |
| 87 | 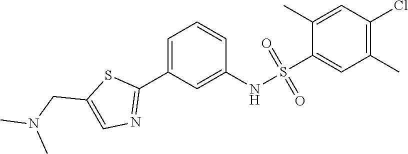 | 4-chloro-N-(3-(5- ((dimethylamino)methyl)thiazol-2- yl)phenyl)-2,5- dimethylbenzenesulfonamide |
| 88 | 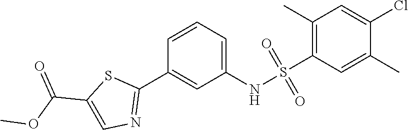 | methyl 2-(3-((4-chloro-2,5- dimethylphenyl)sulfonamido)phenyl) thiazole-5-carboxylate |
| 89 | 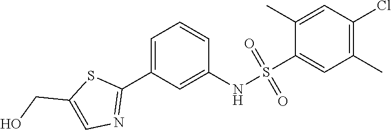 [SMILES: Cc1cc(S(=O)(=O)Nc2cccc(-c3ncc(CO)s3)c2)c(C)cc1Cl] | 4-chloro-N-(3-(5- (hydroxymethyl)thiazol-2-yl)phenyl)- 2,5-dimethylbenzenesulfonamide |
| 90 | 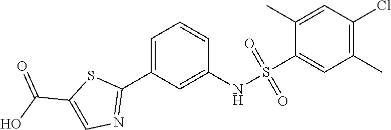 [SMILES: Cc1cc(S(=O)(=O)Nc2cccc(-c3ncc(C(=O)O)s3)c2)c(C)cc1Cl] | 2-(3-((4-chloro-2,5- dimethylphenyl)sulfonamido)phenyl) thiazole-5-carboxylic acid |
| 91 | 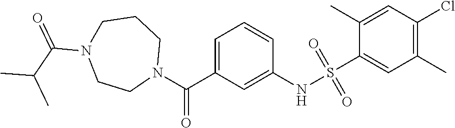 [SMILES: Cc1cc(S(=O)(=O)Nc2cccc(C(=O)N3CCCN(C(=O)C(C)C)CC3)c2)c(C)cc1Cl] | 4-chloro-N-(3-(4-isobutyryl-1,4- diazepane-1-carbonyl)phenyl)-2,5- dimethylbenzenesulfonamide |
| 92 | 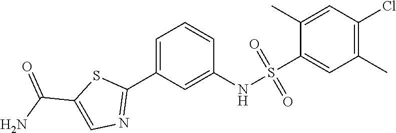 [SMILES: Cc1cc(S(=O)(=O)Nc2cccc(-c3ncc(C(N)=O)s3)c2)c(C)cc1Cl] | 2-(3-((4-chloro-2,5- dimethylphenyl)sulfonamido)phenyl) thiazole-5-carboxamide |
| 93 | 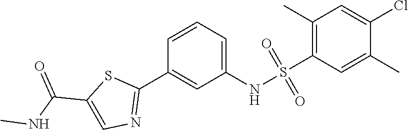 | 2-(3-((4-chloro-2,5- dimethylphenyl)sulfonamido)phenyl)- N-methylthiazole-5-carboxamide |
| 94 | 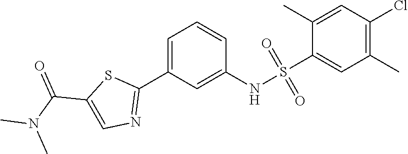 | 2-(3-((4-chloro-2,5- dimethylphenyl)sulfonamido)phenyl)- N,N-dimethylthiazole-5-carboxamide |
| 95 | 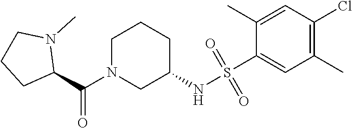 [SMILES: Cc1cc(S(=O)(=O)N[C@H]2CCCN(C(=O)[C@H]3CCCN3C)C2)c(C)cc1Cl] | 4-chloro-2,5-dimethyl-N-((S)-1- (methyl-D-prolyl)piperidin-3- yl)benzenesulfonamide |
| 96 | 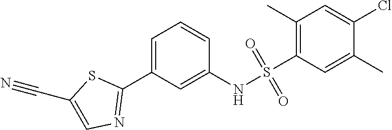 [SMILES: Cc1cc(S(=O)(=O)Nc2cccc(-c3ncc(C#N)s3)c2)c(C)cc1Cl] | 4-chloro-N-(3-(5-cyanothiazol-2- yl)phenyl)-2,5- dimethylbenzenesulfonamide |
| 97 | 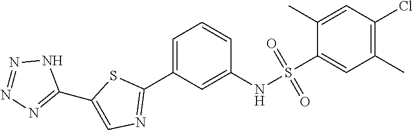 [SMILES: Cc1cc(S(=O)(=O)Nc2cccc(-c3ncc(-c4nnn[nH]4)s3)c2)c(C)cc1Cl] | N-(3-(5-(1H-tetrazol-5-yl)thiazol-2- yl)phenyl)-4-chloro-2,5- dimethylbenzenesulfonamide |
| 98 | 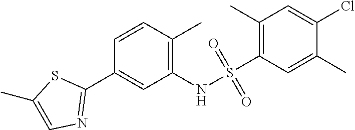 | 4-chloro-2,5-dimethyl-N-(2-methyl-5- (5-methylthiazol-2- yl)phenyl)benzenesulfonamide |
| 99 | 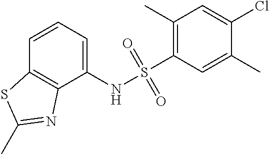 [SMILES: Cc1nc2c(NS(=O)(=O)c3cc(C)c(Cl)cc3C)cccc2s1] | 4-chloro-2,5-dimethyl-N-(2- methylbenzo[d]thiazol-4- yl)benzenesulfonamide |
| 100 | 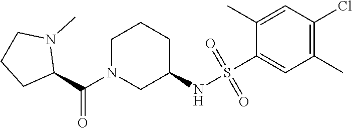 [SMILES: Cc1cc(S(=O)(=O)N[C@@H]2CCCN(C(=O)[C@H]3CCCN3C)C2)c(C)cc1Cl] | 4-chloro-2,5-dimethyl-N-((R)-1- (methyl-D-prolyl)piperidin-3- yl)benzenesulfonamide |
| 101 | 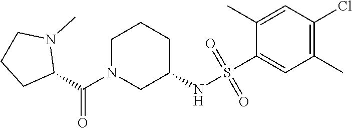 [SMILES: Cc1cc(S(=O)(=O)N[C@H]2CCCN(C(=O)[C@@H]3CCCN3C)C2)c(C)cc1Cl] | 4-chloro-2,5-dimethyl-N-((S)-1- (methyl-L-prolyl)piperidin-3- yl)benzenesulfonamide |
| 102 | 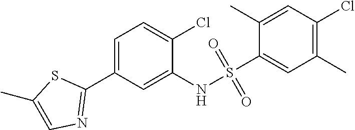 | 4-chloro-N-(2-chloro-5-(5- methylthiazol-2-yl)phenyl)-2,5- dimethylbenzenesulfonamide |
| 103 | 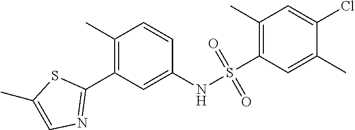 | 4-chloro-2,5-dimethyl-N-(4-methyl-3- (5-methylthiazol-2- yl)phenyl)benzenesulfonamide |
| 104 | 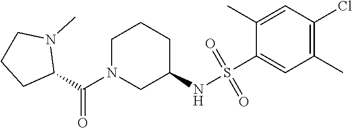 [SMILES: Cc1cc(S(=O)(=O)N[C@@H]2CCCN(C(=O)[C@@H]3CCCN3C)C2)c(C)cc1Cl] | 4-chloro-2,5-dimethyl-N-((R)-1- (methyl-L-prolyl)piperidin-3- yl)benzenesulfonamide |
| 105 | 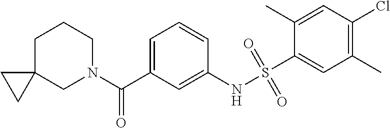 [SMILES: Cc1cc(S(=O)(=O)Nc2cccc(C(=O)N3CCCC4(CC4)C3)c2)c(C)cc1Cl] | N-(3-(5-azaspiro[2.5]octane-5- carbonyl)phenyl)-4-chloro-2,5- dimethylbenzenesulfonamide |
| 106 | 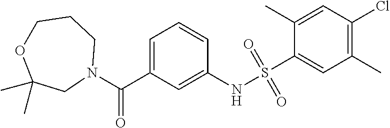 [SMILES: Cc1cc(S(=O)(=O)Nc2cccc(C(=O)N3CCCOC(C)(C)C3)c2)c(C)cc1Cl] | 4-chloro-N-(3-(2,2-dimethyl-1,4- oxazepane-4-carbonyl)phenyl)-2,5- dimethylbenzenesulfonamide |
| 107 | 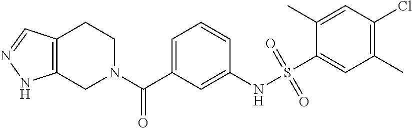 [SMILES: Cc1cc(S(=O)(=O)Nc2cccc(C(=O)N3CCc4cn[nH]c4C3)c2)c(C)cc1Cl] | 4-chloro-2,5-dimethyl-N-(3-(4,5,6,7- tetrahydro-1H-pyrazolo[3,4-c]pyridine- 6-carbonyl)phenyl)benzenesulfonamide |
| 108 | 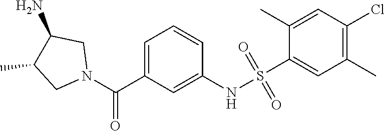 [SMILES: Cc1cc(S(=O)(=O)Nc2cccc(C(=O)N3C[C@H](C)[C@@H](N)C3)c2)c(C)cc1Cl] | N-(3-((3R,4S)-3-amino-4- methylpyrrolidine-1-carbonyl)phenyl)- 4-chloro-2,5- dimethylbenzenesulfonamide |
| 109 | 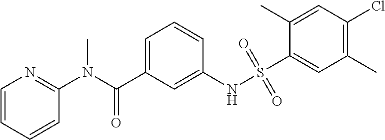 [SMILES: Cc1cc(S(=O)(=O)Nc2cccc(C(=O)N(C)c3ccccn3)c2)c(C)cc1Cl] | 3-((4-chloro-2,5- dimethylphenyl)sulfonamido)-N- methyl-N-(pyridin-2-yl)benzamide |
| 110 | 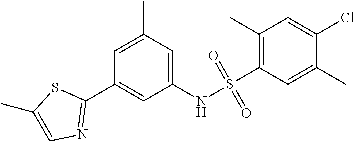 | 4-chloro-2,5-dimethyl-N-(3-methyl-5- (5-methylthiazol-2- yl)phenyl)benzenesulfonamide |
| 111 | 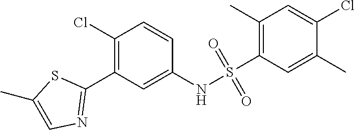 | 4-chloro-N-(4-chloro-3-(5- methylthiazol-2-yl)phenyl)-2,5- dimethylbenzenesulfonamide |
| 112 | 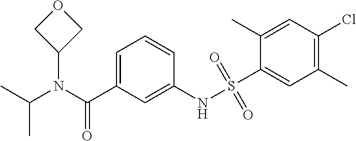 [SMILES: Cc1cc(S(=O)(=O)Nc2cccc(C(=O)N(C(C)C)C3COC3)c2)c(C)cc1Cl] | 3-((4-chloro-2,5- dimethylphenyl)sulfonamido)-N- isopropyl-N-(oxetan-3-yl)benzamide |
| 113 | 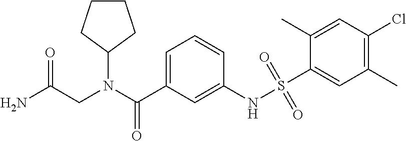 [SMILES: Cc1cc(S(=O)(=O)Nc2cccc(C(=O)N(CC(N)=O)C3CCCC3)c2)c(C)cc1Cl] | N-(2-amino-2-oxoethyl)-3-((4-chloro- 2,5-dimethylphenyl)sulfonamido)-N- cyclopentylbenzamide |
| 114 | 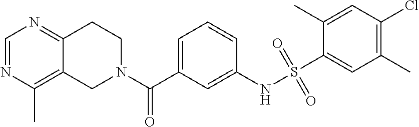 [SMILES: Cc1cc(S(=O)(=O)Nc2cccc(C(=O)N3CCc4ncnc(C)c4C3)c2)c(C)cc1Cl] | 4-chloro-2,5-dimethyl-N-(3-(4- methyl-5,6,7,8-tetrahydropyrido [4,3-d]pyrimidine-6-carbonyl) phenyl)benzenesulfonamide |
| 115 | 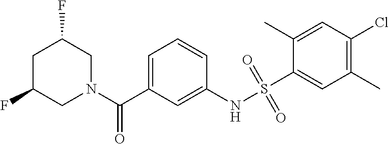 [SMILES: Cc1cc(S(=O)(=O)Nc2cccc(C(=O)N3C[C@@H](F)C[C@H](F)C3)c2)c(C)cc1Cl] | 4-chloro-N-(3-((3S,5S)-3,5- difluoropiperidine-1-carbonyl)phenyl)- 2,5-dimethylbenzenesulfonamide |
| 116 | 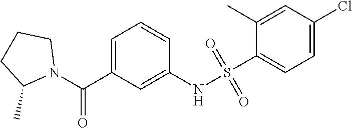 [SMILES: Cc1cc(Cl)ccc1S(=O)(=O)Nc1cccc(C(=O)N2CCC[C@H]2C)c1] | (R)-4-chloro-2-methyl-N-(3-(2- methylpyrrolidine-1- carbonyl)phenyl)benzenesulfonamide |
| 117 | 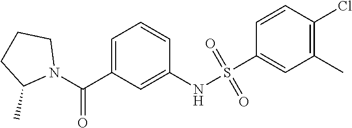 [SMILES: Cc1cc(S(=O)(=O)Nc2cccc(C(=O)N3CCC[C@H]3C)c2)ccc1Cl] | (R)-4-chloro-3-methyl-N-(3-(2- methylpyrrolidine-1- carbonyl)phenyl)benzenesulfonamide |
| 118 | 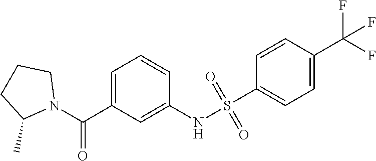 [SMILES: C[C@@H]1CCCN1C(=O)c1cccc(NS(=O)(=O)c2ccc(C(F)(F)F)cc2)c1] | (R)-N-(3-(2-methylpyrrolidine-1- carbonyl)phenyl)-4- (trifluoromethyl)benzenesulfonamide |
| 119 | 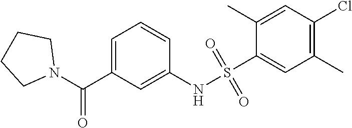 [SMILES: Cc1cc(S(=O)(=O)Nc2cccc(C(=O)N3CCCC3)c2)c(C)cc1Cl] | 4-chloro-2,5-dimethyl-N-(3- (pyrrolidine-1-carbonyl)phenyl) benzenesulfonamide |
| 120 | 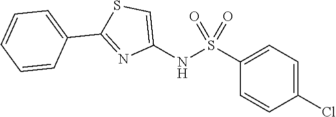 [SMILES: O=S(=O)(Nc1csc(-c2ccccc2)n1)c1ccc(Cl)cc1] | 4-chloro-N-(2-phenylthiazol-4- yl)benzenesulfonamide |
| 121 | 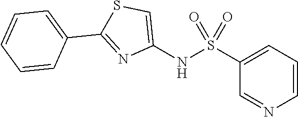 [SMILES: O=S(=O)(Nc1csc(-c2ccccc2)n1)c1cccnc1] | N-(2-phenylthiazol-4-yl)pyridine-3- sulfonamide |
| 122 | 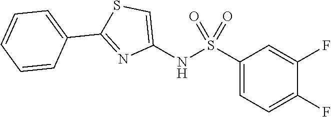 [SMILES: O=S(=O)(Nc1csc(-c2ccccc2)n1)c1ccc(F)c(F)c1] | 3,4-difluoro-N-(2-phenylthiazol-4- yl)benzenesulfonamide |
| 123 | 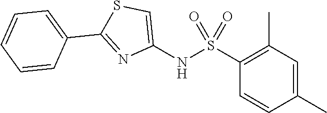 [SMILES: Cc1ccc(S(=O)(=O)Nc2csc(-c3ccccc3)n2)c(C)c1] | 2,4-dimethyl-N-(2-phenylthiazol-4- yl)benzenesulfonamide |
| 124 | 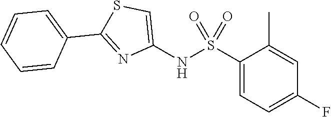 [SMILES: Cc1cc(F)ccc1S(=O)(=O)Nc1csc(-c2ccccc2)n1] | 4-fluoro-2-methyl-N-(2-phenylthiazol- 4-yl)benzenesulfonamide |
| 125 | 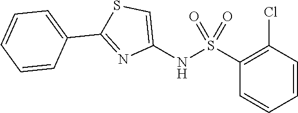 [SMILES: O=S(=O)(Nc1csc(-c2ccccc2)n1)c1ccccc1Cl] | 2-chloro-N-(2-phenylthiazol-4- yl)benzenesulfonamide |
| 126 | 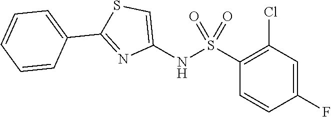 [SMILES: O=S(=O)(Nc1csc(-c2ccccc2)n1)c1ccc(F)cc1Cl] | 2-chloro-4-fluoro-N-(2-phenylthiazol- 4-yl)benzenesulfonamide |
| 127 | 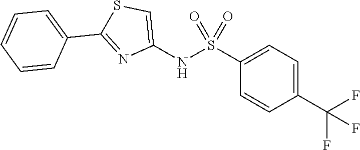 [SMILES: O=S(=O)(Nc1csc(-c2ccccc2)n1)c1ccc(C(F)(F)F)cc1] | N-(2-phenylthiazol-4-yl)-4- (trifluoromethyl)benzenesulfonamide |
| 128 | 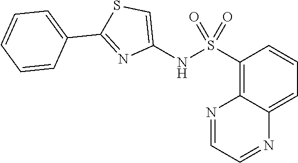 [SMILES: O=S(=O)(Nc1csc(-c2ccccc2)n1)c1cccc2nccnc12] | N-(2-phenylthiazol-4-yl)quinoxaline- 5-sulfonamide |
| 129 | 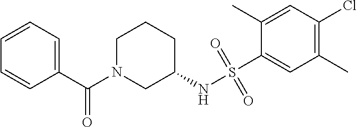 [SMILES: Cc1cc(S(=O)(=O)N[C@H]2CCCN(C(=O)c3ccccc3)C2)c(C)cc1Cl] | (S)-N-(1-benzoylpiperidin-3-yl)-4- chloro-2,5-dimethylbenzenesulfonamide |
| 130 | 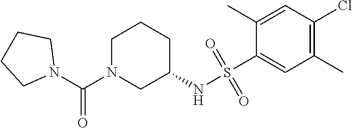 [SMILES: Cc1cc(S(=O)(=O)N[C@H]2CCCN(C(=O)N3CCCC3)C2)c(C)cc1Cl] | (S)-4-chloro-2,5-dimethyl-N-(1- (pyrrolidinee-1-carbonyl)piperidin-3- yl)benzenesulfonamide |
| 131 | 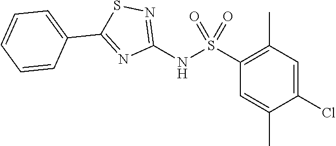 [SMILES: Cc1cc(S(=O)(=O)Nc2nsc(-c3ccccc3)n2)c(C)cc1Cl] | 4-chloro-2,5-dimethyl-N-(5-phenyl- 1,2,4-thiadiazol-3- yl)benzenesulfonamide |
| 132 | 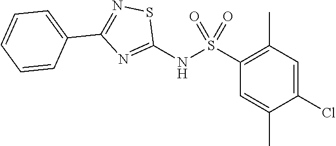 [SMILES: Cc1cc(S(=O)(=O)Nc2nc(-c3ccccc3)ns2)c(C)cc1Cl] | 4-chloro-2,5-dimethyl-N-(3-phenyl- 1,2,4-thiadiazol-5- yl)benzenesulfonamide |
| 133 | 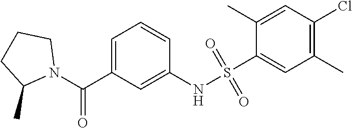 [SMILES: Cc1cc(S(=O)(=O)Nc2cccc(C(=O)N3CCC[C@@H]3C)c2)c(C)cc1Cl] | (S)-4-chloro-2,5-dimethyl-N-(3-(2- methylpyrrolidine-1- carbonyl)phenyl)benzenesulfonamide |
| 134 | 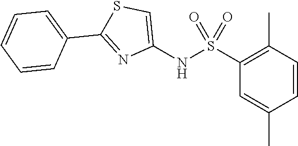 [SMILES: Cc1ccc(C)c(S(=O)(=O)Nc2csc(-c3ccccc3)n2)c1] | 2,5-dimethyl-N-(2-phenylthiazol-4- yl)benzenesulfonamide |
| 135 | 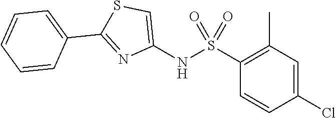 [SMILES: Cc1cc(Cl)ccc1S(=O)(=O)Nc1csc(-c2ccccc2)n1] | 4-chloro-2-methyl-N-(2-phenylthiazol- 4-yl)benzenesulfonamide |
| 136 | 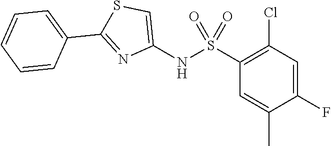 [SMILES: Cc1cc(S(=O)(=O)Nc2csc(-c3ccccc3)n2)c(Cl)cc1F] | 2-chloro-4-fluoro-5-methyl-N-(2- phenylthiazol-4-yl)benzenesulfonamide |
| 137 | 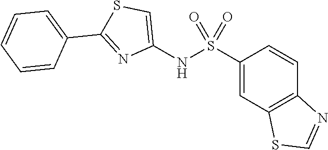 [SMILES: O=S(=O)(Nc1csc(-c2ccccc2)n1)c1ccc2ncsc2c1] | N-(2-phenylthiazol-4- yl)benzo[d]thiazole-6-sulfonamide |
| 138 | 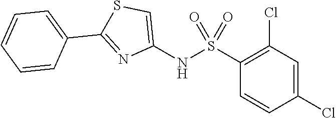 [SMILES: O=S(=O)(Nc1csc(-c2ccccc2)n1)c1ccc(Cl)cc1Cl] | 2,4-dichloro-N-(2-phenylthiazol-4- yl)benzenesulfonamide |
| 139 | 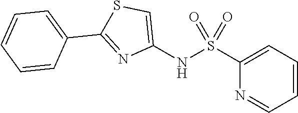 [SMILES: O=S(=O)(Nc1csc(-c2ccccc2)n1)c1ccccn1] | N-(2-phenylthiazol-4-yl)pyridine-2- sulfonamide |
| 140 | 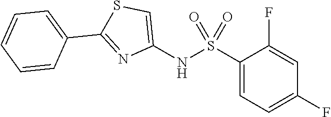 [SMILES: O=S(=O)(Nc1csc(-c2ccccc2)n1)c1ccc(F)cc1F] | 2,4-difluoro-N-(2-phenylthiazol-4- yl)benzenesulfonamide |
| 141 | 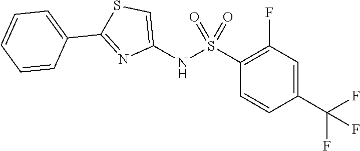 [SMILES: O=S(=O)(Nc1csc(-c2ccccc2)n1)c1ccc(C(F)(F)F)cc1F] | 2-fluoro-N-(2-phenylthiazol-4-yl)-4- (trifluoromethyl)benzenesulfonamide |
| 142 | 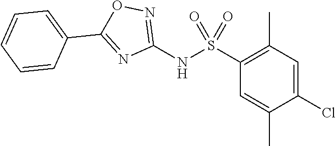 [SMILES: Cc1cc(S(=O)(=O)Nc2noc(-c3ccccc3)n2)c(C)cc1Cl] | 4-chloro-2,5-dimethyl-N-(5-phenyl- 1,2,4-oxadiazol-3- yl)benzenesulfonamide |
| 143 | 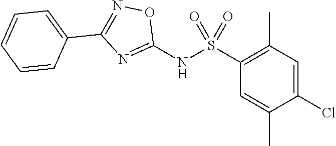 [SMILES: Cc1cc(S(=O)(=O)Nc2nc(-c3ccccc3)no2)c(C)cc1Cl] | 4-chloro-2,5-dimethyl-N-(3-phenyl- 1,2,4-oxadiazol-5- yl)benzenesulfonamide |
| 144 | 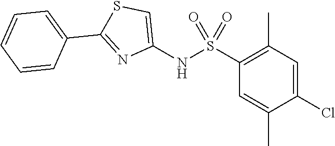 [SMILES: Cc1cc(S(=O)(=O)Nc2csc(-c3ccccc3)n2)c(C)cc1Cl] | 4-chloro-2,5-dimethyl-N-(4- phenylthiazol-2-yl)benzenesulfonamide |
| 145 | 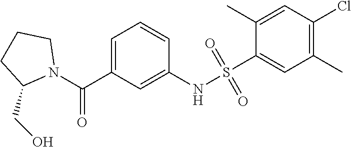 [SMILES: Cc1cc(S(=O)(=O)Nc2cccc(C(=O)N3CCC[C@H]3CO)c2)c(C)cc1Cl] | (S)-4-chloro-N-(3-(2- (hydroxymethyl)pyrrolidine-1- carbonyl)phenyl)-2,5- dimethylbenzenesulfonamide |
| 146 | 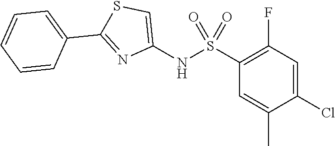 [SMILES: Cc1cc(S(=O)(=O)Nc2csc(-c3ccccc3)n2)c(F)cc1Cl] | 4-chloro-2-fluoro-5-methyl-N-(2- phenylthiazol-4-yl)benzenesulfonamide |
| 147 | 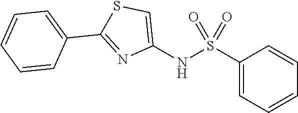 [SMILES: O=S(=O)(Nc1csc(-c2ccccc2)n1)c1ccccc1] | N-(2-phenylthiazol-4- yl)benzenesulfonamide |
| 148 | 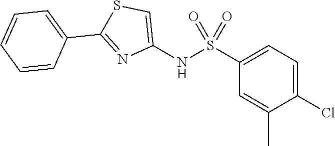 [SMILES: Cc1cc(S(=O)(=O)Nc2csc(-c3ccccc3)n2)ccc1Cl] | 4-chloro-3-methyl-N-(2-phenylthiazol- 4-yl)benzenesulfonamide |
| 149 | 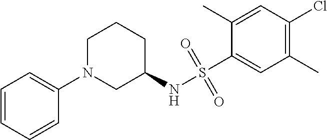 [SMILES: Cc1cc(S(=O)(=O)N[C@@H]2CCCN(c3ccccc3)C2)c(C)cc1Cl] | (R)-4-chloro-2,5-dimethyl-N-(1- phenylpiperidin-3- yl)benzenesulfonamide |
| 150 | 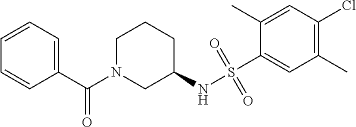 [SMILES: Cc1cc(S(=O)(=O)N[C@@H]2CCCN(C(=O)c3ccccc3)C2)c(C)cc1Cl] | (R)-N-(1-benzoylpiperidin-3-yl)-4- chloro-2,5-dimethylbenzenesulfonamide |
| 151 | 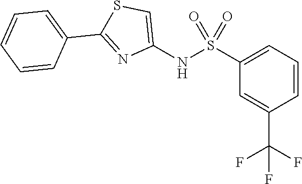 [SMILES: O=S(=O)(Nc1csc(-c2ccccc2)n1)c1cccc(C(F)(F)F)c1] | N-(2-phenylthiazol-4-yl)-3- (trifluoromethyl)benzenesulfonamide |
| 152 | 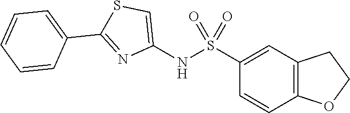 [SMILES: O=S(=O)(Nc1csc(-c2ccccc2)n1)c1ccc2c(c1)CCO2] | N-(2-phenylthiazol-4-yl)-2,3- dihydrobenzofuran-5-sulfonamide |
| 153 | 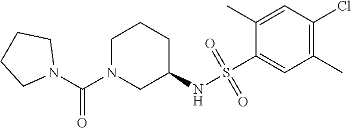 [SMILES: Cc1cc(S(=O)(=O)N[C@@H]2CCCN(C(=O)N3CCCC3)C2)c(C)cc1Cl] | (R)-4-chloro-2,5-dimethyl-N-(1- (pyrrolidine-1-carbonyl)piperidin-3- yl)benzenesulfonamide |
| 154 | 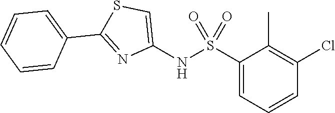 [SMILES: Cc1c(Cl)cccc1S(=O)(=O)Nc1csc(-c2ccccc2)n1] | 3-chloro-2-methyl-N-(2-phenylthiazol- 4-yl)benzenesulfonamide |
| 155 | 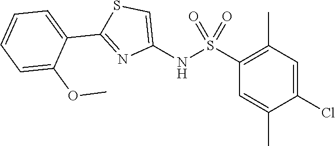 [SMILES: COc1ccccc1-c1nc(NS(=O)(=O)c2cc(C)c(Cl)cc2C)cs1] | 4-chloro-N-(2-(2- methoxyphenyl)thiazol-4-yl)-2,5- dimethylbenzenesulfonamide |
| 156 | 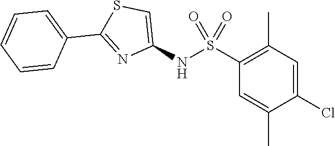 | (R)-4-chloro-2,5-dimethyl-N-(1- phenylpyrrolidin-3- yl)benzenesulfonamide |
| 157 | 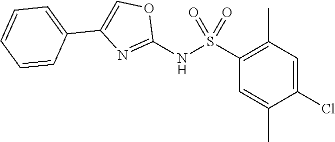 [SMILES: Cc1cc(S(=O)(=O)Nc2nc(-c3ccccc3)co2)c(C)cc1Cl] | 4-chloro-2,5-dimethyl-N-(4- phenyloxazol-2-yl)benzenesulfonamide |
| 158 | 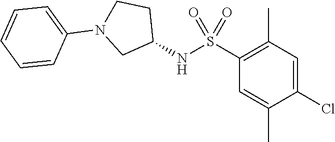 [SMILES: Cc1cc(S(=O)(=O)N[C@H]2CCN(c3ccccc3)C2)c(C)cc1Cl] | (S)-4-chloro-2,5-dimethyl-N-(1- phenylpyrrolidin-3- yl)benzenesulfonamide |
| 159 | 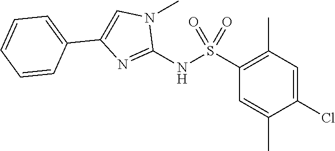 [SMILES: Cc1cc(S(=O)(=O)Nc2nc(-c3ccccc3)cn2C)c(C)cc1Cl] | 4-chloro-2,5-dimethyl-N-(1-methyl-4- phenyl-1H-imidazol-2- yl)benzenesulfonamide |
| 160 | 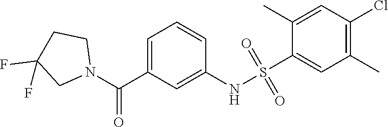 [SMILES: Cc1cc(S(=O)(=O)Nc2cccc(C(=O)N3CCC(F)(F)C3)c2)c(C)cc1Cl] | 4-chloro-N-(3-(3,3-difluoropyrrolidine- 1-carbonyl)phenyl)-2,5- dimethylbenzenesulfonamide |
| 161 | 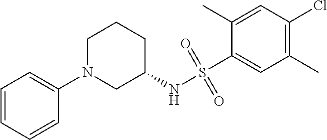 [SMILES: Cc1cc(S(=O)(=O)N[C@H]2CCCN(c3ccccc3)C2)c(C)cc1Cl] | (S)-4-chloro-2,5-dimethyl-N-(1- phenylpiperidin-3- yl)benzenesulfonamide |
| 162 | 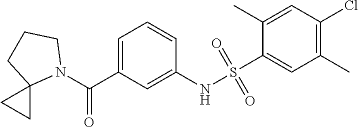 [SMILES: Cc1cc(S(=O)(=O)Nc2cccc(C(=O)N3CCCC34CC4)c2)c(C)cc1Cl] | N-(3-(4-azaspiro[2.4]heptane-4- carbonyl)phenyl)-4-chloro-2,5- dimethylbenzenesulfonamide |
| 163 | 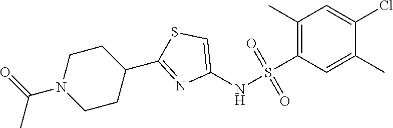 [SMILES: CC(=O)N1CCC(c2nc(NS(=O)(=O)c3cc(C)c(Cl)cc3C)cs2)CC1] | N-(2-(1-acetylpiperidin-4-yl)thiazol-4- yl)-4-chloro-2,5- dimethylbenzenesulfonamide |
| 164 | 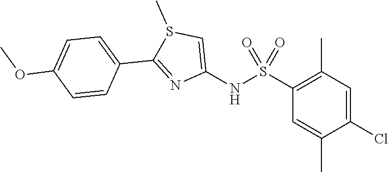 | 4-chloro-N-(2-(4- methoxyphenyl)thiazol-4-yl)-2,5- dimethylbenzenesulfonamide |
| 165 | 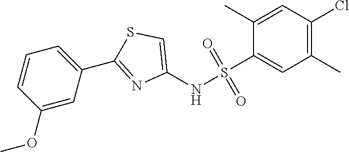 [SMILES: COc1cccc(-c2nc(NS(=O)(=O)c3cc(C)c(Cl)cc3C)cs2)c1] | 4-chloro-N-(2-(3- methoxyphenyl)thiazol-4-yl)-2,5- dimethylbenzenesulfonamide |
| 166 | 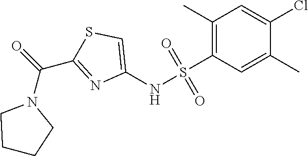 [SMILES: Cc1cc(S(=O)(=O)Nc2csc(C(=O)N3CCCC3)n2)c(C)cc1Cl] | 4-chloro-2,5-dimethyl-N-(2- (pyrrolidine-1-carbonyl)thiazol-4- yl)benzenesulfonamide |
| 167 | 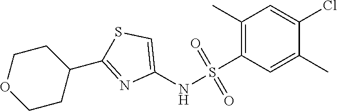 [SMILES: Cc1cc(S(=O)(=O)Nc2csc(C3CCOCC3)n2)c(C)cc1Cl] | 4-chloro-2,5-dimethyl-N-(2-(tetrahydro- 2H-pyran-4-yl)thiazol-4- yl)benzenesulfonamide |
| 168 | 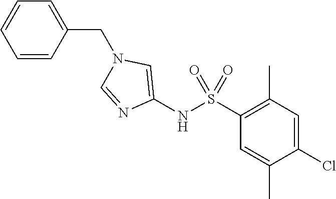 [SMILES: Cc1cc(S(=O)(=O)Nc2cn(Cc3ccccc3)cn2)c(C)cc1Cl] | N-(1-benzyl-1H-imidazol-4-yl)-4- chloro-2,5-dimethylbenzenesulfonamide |
| 169 | 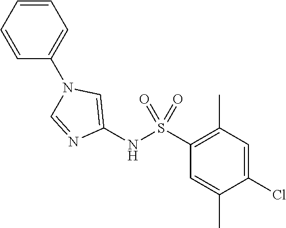 [SMILES: Cc1cc(S(=O)(=O)Nc2cn(-c3ccccc3)cn2)c(C)cc1Cl] | 4-chloro-2,5-dimethyl-N-(1-phenyl-1H- imidazol-4-yl)benzenesulfonamide |
| 170 | 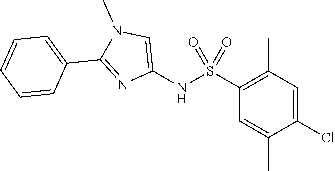 [SMILES: Cc1cc(S(=O)(=O)Nc2cn(C)c(-c3ccccc3)n2)c(C)cc1Cl] | 4-chloro-2,5-dimethyl-N-(1-methyl-2- phenyl-1H-imidazol-4- yl)benzenesulfonamide |
| 171 | 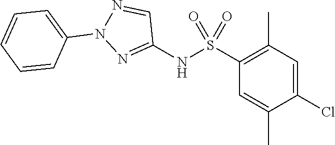 [SMILES: Cc1cc(S(=O)(=O)Nc2cnn(-c3ccccc3)n2)c(C)cc1Cl] | 4-chloro-2,5-dimethyl-N-(2-phenyl-2H- 1,2,3-triazol-4-yl)benzenesulfonamide |
| 172 | 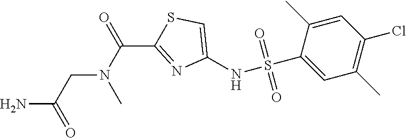 [SMILES: Cc1cc(S(=O)(=O)Nc2csc(C(=O)N(C)CC(N)=O)n2)c(C)cc1Cl] | N-(2-amino-2-oxoethyl)-4-((4-chloro- 2,5-dimethylphenyl)sulfonamido)-N- methylthiazole-2-carboxamide |
| 173 | 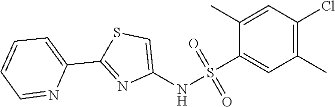 [SMILES: Cc1cc(S(=O)(=O)Nc2csc(-c3ccccn3)n2)c(C)cc1Cl] | 4-chloro-2,5-dimethyl-N-(2-(pyridin-2- yl)thiazol-4-yl)benzenesulfonamide |
| 174 | 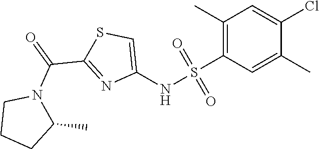 [SMILES: Cc1cc(S(=O)(=O)Nc2csc(C(=O)N3CCC[C@H]3C)n2)c(C)cc1Cl] | (R)-4-chloro-2,5-dimethyl-N-(2-(2- methylpyrrolidine-1-carbonyl)thiazol-4- yl)benzenesulfonamide |
| 175 | 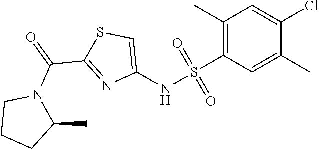 [SMILES: Cc1cc(S(=O)(=O)Nc2csc(C(=O)N3CCC[C@@H]3C)n2)c(C)cc1Cl] | (S)-4-chloro-2,5-dimethyl-N-(2-(2- methylpyrrolidine-1-carbonyl)thiazol-4- yl)benzenesulfonamide |
| 176 | 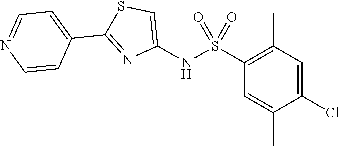 [SMILES: Cc1cc(S(=O)(=O)Nc2csc(-c3ccncc3)n2)c(C)cc1Cl] | 4-chloro-2,5-dimethyl-N-(2-(pyridin-4- yl)thiazol-4-yl)benzenesulfonamide |
| 177 | 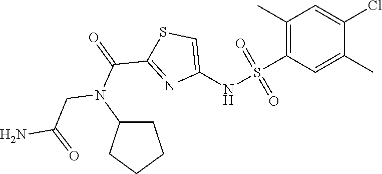 [SMILES: Cc1cc(S(=O)(=O)Nc2csc(C(=O)N(CC(N)=O)C3CCCC3)n2)c(C)cc1Cl] | N-(2-amino-2-oxoethyl)-4-((4-chloro- 2,5-dimethylphenyl)sulfonamido)-N- cyclopentylthiazole-2-carboxamide |
| 178 | 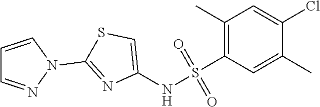 [SMILES: Cc1cc(S(=O)(=O)Nc2csc(-n3cccn3)n2)c(C)cc1Cl] | N-(2-(1H-pyrazol-1-yl)thiazol-4-yl)-4- chloro-2,5-dimethylbenzenesulfonamide |
| 179 | 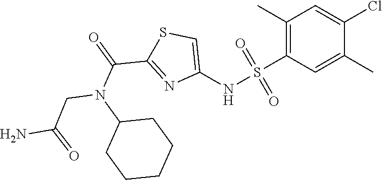 [SMILES: Cc1cc(S(=O)(=O)Nc2csc(C(=O)N(CC(N)=O)C3CCCCC3)n2)c(C)cc1Cl] | N-(2-amino-2-oxoethyl)-4-((4-chloro- 2,5-dimethylphenyl)sulfonamido)-N- cyclohexylthiazole-2-carboxamide |
| 180 | 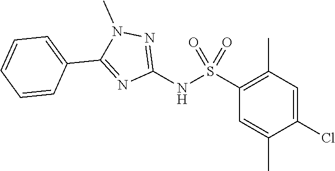 [SMILES: Cc1cc(S(=O)(=O)Nc2nc(-c3ccccc3)n(C)n2)c(C)cc1Cl] | 4-chloro-2,5-dimethyl-N-(1-methyl-5- phenyl-1H-1,2,4-triazol-3- yl)benzenesulfonamide |
| 181 | 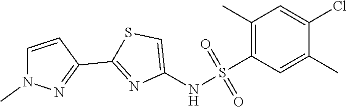 [SMILES: Cc1cc(S(=O)(=O)Nc2csc(-c3ccn(C)n3)n2)c(C)cc1Cl] | 4-chloro-2,5-dimethyl-N-(2-(1-methyl- 1H-pyrazol-3-yl)thiazol-4- yl)benzenesulfonamide |
| 182 | 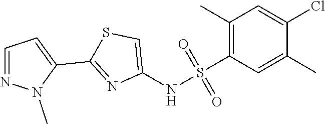 [SMILES: Cc1cc(S(=O)(=O)Nc2csc(-c3ccnn3C)n2)c(C)cc1Cl] | 4-chloro-2,5-dimethyl-N-(2-(1-methyl- 1H-pyrazol-5-yl)thiazol-4- yl)benzenesulfonamide |
| 183 | 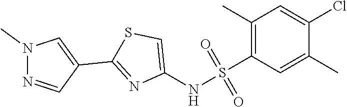 [SMILES: Cc1cc(S(=O)(=O)Nc2csc(-c3cnn(C)c3)n2)c(C)cc1Cl] | 4-chloro-2,5-dimethyl-N-(2-(1-methyl- 1H-pyrazol-4-yl)thiazol-4- yl)benzenesulfonamide |
| 184 | 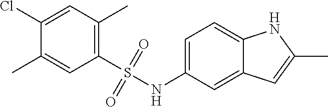 | 4-chloro-2,5-dimethyl-N-(2-methyl-1H- indol-5-yl)benzenesulfonamide |
| 185 | 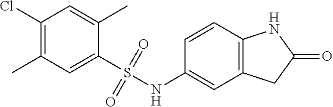 [SMILES: Cc1cc(S(=O)(=O)Nc2ccc3c(c2)CC(=O)N3)c(C)cc1Cl] | 4-chloro-2,5-dimethyl-N-(2-oxoindolin- 5-yl)benzenesulfonamide |
| 186 | 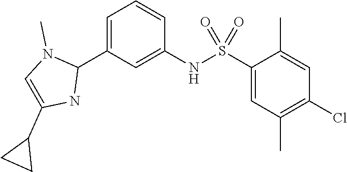 [SMILES: Cc1cc(S(=O)(=O)Nc2cccc(C3NC(C4CC4)=CN3C)c2)c(C)cc1Cl] | 4-chloro-N-(3-(4-cyclopropyl-1-methyl- 1H-imidazol-2-yl)phenyl)-2,5- dimethylbenzenesulfonamide |
| 187 | 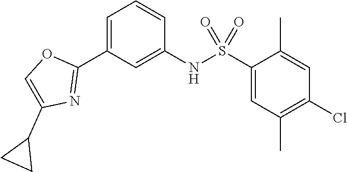 [SMILES: Cc1cc(S(=O)(=O)Nc2cccc(-c3nc(C4CC4)co3)c2)c(C)cc1Cl] | 4-chloro-N-(3-(4-cyclopropyloxazol-2- yl)phenyl)-2,5- dimethylbenzenesulfonamide |
| 188 | 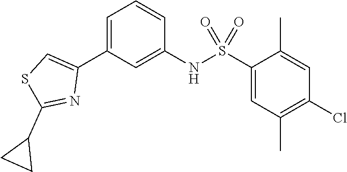 [SMILES: Cc1cc(S(=O)(=O)Nc2cccc(-c3csc(C4CC4)n3)c2)c(C)cc1Cl] | 4-chloro-N-(3-(2-cyclopropylthiazol-4- yl)phenyl)-2,5- dimethylbenzenesulfonamide |
| 189 | 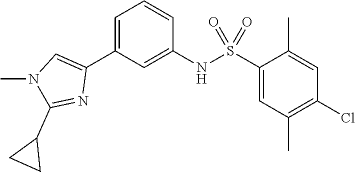 [SMILES: Cc1cc(S(=O)(=O)Nc2cccc(-c3cn(C)c(C4CC4)n3)c2)c(C)cc1Cl] | 4-chloro-N-(3-(2-cyclopropyl-1-methyl- 1H-imidazol-4-yl)phenyl)-2,5- dimethylbenzenesulfonamide |
| 190 | 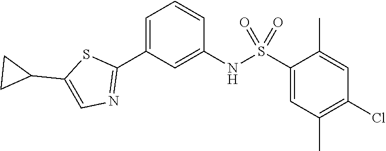 [SMILES: Cc1cc(S(=O)(=O)Nc2cccc(-c3ncc(C4CC4)s3)c2)c(C)cc1Cl] | 4-chloro-N-(3-(5-cyclopropylthiazol-2- yl)phenyl)-2,5- dimethylbenzenesulfonamide, or a pharmaceutically acceptable salt thereof: |
| 191 | 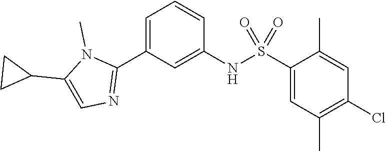 [SMILES: Cc1cc(S(=O)(=O)Nc2cccc(-c3ncc(C4CC4)n3C)c2)c(C)cc1Cl] | 4-chloro-N-(3-(5-cyclopropyl-1-methyl- 1H-imidazol-2-yl)phenyl)-2,5- dimethylbenzenesulfonamide |
| 192 | 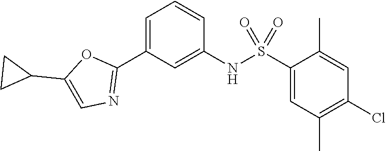 [SMILES: Cc1cc(S(=O)(=O)Nc2cccc(-c3ncc(C4CC4)o3)c2)c(C)cc1Cl] | 4-chloro-N-(3-(5-cyclopropyloxazol-2- yl)phenyl)-2,5- dimethylbenzenesulfonamide |
| 193 | 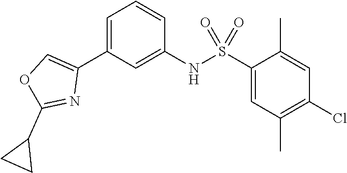 [SMILES: Cc1cc(S(=O)(=O)Nc2cccc(-c3coc(C4CC4)n3)c2)c(C)cc1Cl] | 4-chloro-N-(3-(2-cyclopropyloxazol-4- yl)phenyl)-2,5- dimethylbenzenesulfonamide |

In certain examples of formula (I), the compound, or a pharmaceutically acceptable salt thereof, is selected from the group consisting of:

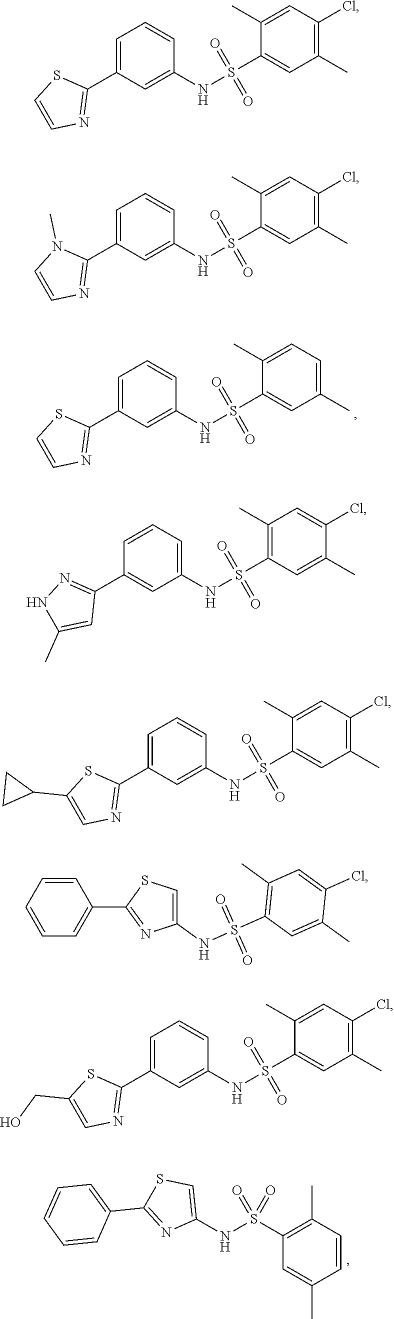

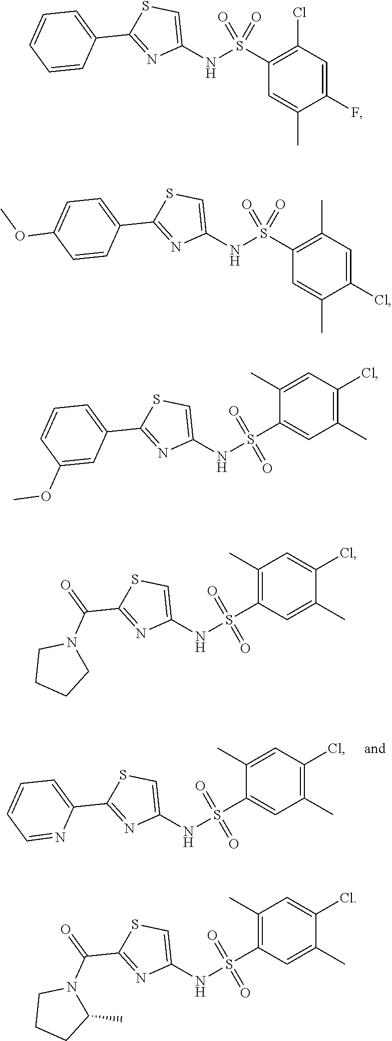

In certain examples, the compounds modulate POLγ.

In certain examples, the compounds inhibit POLγ.

In certain examples, the compounds promote POLγ.

The compounds of the present invention may contain asymmetric carbon atoms (sometimes as the result of a deuterium atom) and thereby can exist as either individual stereoisomers or mixtures of the enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. Carbon atoms labelled with * or ** refer to a compound that is chiral but the absolute stereochemistry has not been determined.

The compounds of the present invention may contain double bonds that may exist in more than one geometric isomer. Examples of such double bonds are carbon-carbon double bonds which form alkenes. In the case of carbon-carbon double bonds, the geometric isomers may be E or Z isomers.

Unless otherwise indicated, when a disclosed compound is named or depicted by a structure without specifying the geometric isomerism and has one or more possible geometric isomers, it is understood to represent all possible geometric isomers of the compound.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and ketoenol tautomerism is possible, it is understood that the substituent might in fact be present, in whole or in part, in the oxo (═O) form.

Compounds of the present invention may exist in amorphous form and/or one or more crystalline forms. As such all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable Isotopic substitution of the deuterated compounds of the invention. See, e.g., Gannes, L. Z., et al., Comp Biochem. Physiol. Mol. Integr Physiol., 119:725 (1998). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium. See Pirali, T. et al., J. Med. Chem., 62:5276-97 (2019). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart. See Fukuto, J. M., et al., J. Med. Chem., 34:2871-76 (1991). Because some drugs have multiple sites of metabolism or more than one active sites for binding to a target, it is unpredictable as to which sites may benefit by deuterium replacement or to what extent isotope enrichment is necessary to produce a beneficial effect.

### Preparation of the Compounds

The starting materials and reagents used in each step in the preparation are known and can be readily prepared or purchased from commercial sources.

The compound obtained in each step can also be used for the next reaction as a reaction mixture thereof or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min to 48 h, preferably 10 min to 8 h.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz, F. T., Theophil E., Ulf D., Andreas S., Schutzenmeister, N.) Wiley, 2015; “Organic Syntheses Collective Volumes 1-12” (John Wiley & Sons Inc.); “Comprehensive Organic Transformations, Third Edition” (Larock R. C.) Wiley, 2018, and the like.

In each step, protection or deprotection of a functional group is performed by a known method, for example, the methods described in “Protective Groups in Organic Synthesis, 4^{th} Ed.” (Greene, T. W., Wuts, P. G. M.) Wiley-Interscience, 2007; “Protecting Groups 3rd Ed.” (Kocienski, P. J.) Thieme, 2004, and the like.

Deuterated POLγ modulators of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

### Compositions

The present invention further includes pharmaceutical compositions of the compounds, a pharmaceutically acceptable salt of said compounds, or prodrugs of said compounds. In addition to the compound of the invention, a salt thereof, or a prodrug thereof, the pharmaceutical compositions comprise one or more pharmaceutically acceptable excipients, such excipients being compatible with other ingredients in the composition and also being not toxic or otherwise harmful. Examples of excipients include carriers, lubricants, binders, disintegrants, solvents, solubilizing agents, suspending agents, isotonic agents, buffers, soothing agents, preservatives, antioxidants, colorants, taste-modifying agents, absorbents, and/or wetting agents.

The pharmaceutical compositions of the invention include those suitable for oral, rectal, nasal, topical, buccal, sublingual, vaginal or parenteral (including subcutaneous, intramuscular, intravenous and intradermal) administration. Such compositions may be prepared by any methods well known in the art of pharmaceutical formulations and pharmacy. See, e.g., Remington: The Science and Practice of Pharmacy, Elsevier Science, 23rd ed. (2020).

A pharmaceutically acceptable excipients can contain physiologically acceptable agents that act, for example, to stabilize, increase solubility or to increase the absorption of a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation of composition can be a self-emulsifying drug delivery system or a self-microemulsifying drug delivery system. The composition (preparation) also can be a liposome or other polymer matrix, which can have incorporated therein, for example, the compound of the present disclosure. Liposomes, for example, which comprise phospholipids or other lipids, are nontoxic, physiologically acceptable and metabolizable carriers that are relatively simple to make and administer.

Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, hydrogels, gelatin, hydrogenated naphthalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to a person of ordinary skill in the art for the manufacture of pharmaceutical compositions. Solid dosage forms for oral administration include capsules (both soft and hard gelatin capsules), tablets, powders, and granules. Generally, these pharmaceutical preparations contain active ingredients admixed with pharmaceutically acceptable excipients. These excipients include, for example, inert diluents, such as calcium carbonate, sodium carbonate, lactose, sucrose, glucose, mannitol, cellulose, starch, calcium phosphate, sodium phosphate, kaolin and the like; binding agents, buffering agents, and/or lubricating agents (e.g., magnesium stearate) may also be used. Tablets and capsules can additionally be prepared with release-controlling coatings such as enteric coatings. The compositions may optionally contain sweetening, flavoring, coloring, perfuming, and preserving agents in order to provide a more palatable preparation.

### Examples

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by mass spectrometry and/or NMR, where peaks assigned to the characteristic protons in the title compound are presented where appropriate. ¹H NMR shift (6) are given in parts per million (ppm) down field from an internal reference standard.

The abbreviations used herein are known to a person of ordinary skill in the art. A partial list of abbreviations that may be used herein include: acetonitrile (CH₃CN), calculated (Calcd.), cesium carbonate (Cs₂CO₃), copper(I) oxide (Cu₂O), degrees Celsius (° C.), deuterium (d), dichloromethane (DCM, CH₂Cl₂), dichloroethane (DCE), N,N-diisopropylethylamine (DIPEA), dimethylformamide (DMF), dimethyl sulfoxide (DMSO), ethyl acetate (EtOAc), ethanol (EtOH), hour (h), N-[(dimethylamino)-1H-1,2,3-triazolo-[4,5-b]pyridin-1-ylmethylene]-N-methylmethanaminium hexafluorophosphate N-oxide (HATU), high performance liquid chromatography (HPLC), lithium hydroxide monohydrate (LiOH·H2O), megahertz (MHz), methanol (MeOH), minutes (min), nuclear magnetic resonance (NMR), potassium carbonate (K₂CO₃), potassium hydroxide (KOH), liquid chromatography-mass spectrometry (LCMS), methanesulfonyl chloride (MsCl), room/ambient temperature (RT), sodium bicarbonate (NaHCO₃), sodium borohydride (NaBH₄), sodium hydride (NaH), sodium sulfate (Na₂SO₃), supercritical fluid chromatography (SFC), tetrahydrofuran (THF), triethylamine (Et₃N, TEA), palladium-tetrakis(triphenylphosphine) (Pd(PPh₃)₄), and water (H₂O).

### Example 1: Synthesis of (R)-2,5-dimethyl-N-(3-(3-methylmorpholine-4-carbonyl)phenyl)benzenesulfonamide

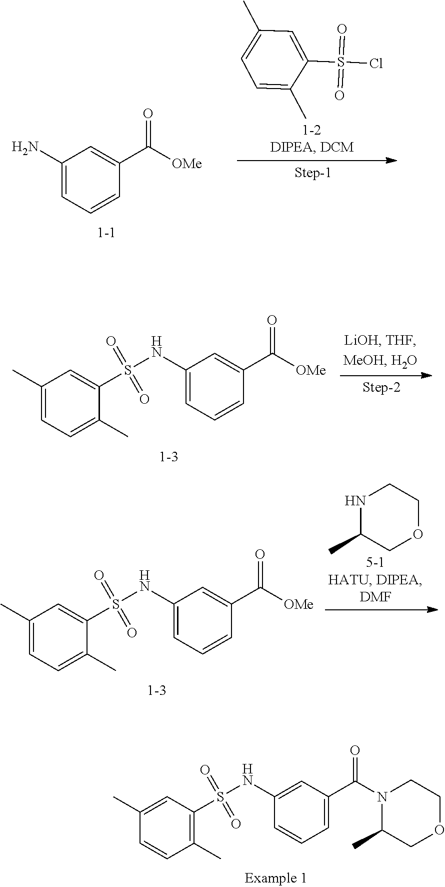

Step-1: methyl 3-((2,5-dimethylphenyl)sulfonamido)benzoate (1-3): To a stirred solution of methyl 3-aminobenzoate 1-1 (0.50 g, 3.31 mmol) in DCM (10 mL), DIPEA (1.2 mL, 6.62 mmol) was added followed by 2,5-dimethylbenzenesulfonyl chloride 1-2 (1.00 g, 4.96 mmol) at 0° C. and the reaction mixture was stirred at RT for 4 h. After completion of the reaction, the reaction mixture was diluted with water, the aqueous layer was extracted with ethyl acetate. The organic layer was dried over anhydrous Na₂SO₄ and concentrated under reduced pressure. The compound was purified by silica gel column chromatography (50% EtOAc/n-hexane) to afford 1-3 (0.62 g). ¹HNMR (400 MHz, DMSO-d₆) δ=10.65 (s, 1H), 7.77-7.65 (m, 2H), 7.55 (d, 1H), 7.40-7.28 (m, 3H), 7.26-7.20 (m, 1H), 3.81 (s, 3H), 2.52 (br s, 3H), 2.30 (s, 3H).

Step-2: 3-((2,5-dimethylphenyl)sulfonamido)benzoic acid (1-4): To a stirred solution of 1-3 (0.62 g, 1.93 mmol) in MeOH (3 mL), THF (5 mL) and water (2 mL), LiOH·H₂O (0.24 g, 5.78 mmol) was added and the reaction mixture was stirred at RT for 8 h. After completion of the reaction, the reaction mixture was concentrated under reduced pressure. The product material was dissolved in water and acidified with 2N HCl to pH 3. The precipitated solid was filtered off, washed with water and dried under reduced pressure to afford 1-4 (0.31 g). ¹H NMR (400 MHz, DMSO-d₆) δ=12.98 (br s, 1H), 10.57 (br s, 1H), 7.76-7.62 (m, 2H), 7.54 (d, 1H), 7.40-7.16 (m, 4H), 2.52 (s, 3H), 2.29 (s, 3H).

Step-3:(R)-2,5-dimethyl-N-(3-(3-methylmorpholine-4-carbonyl)phenyl)benzenesulfonamide (Example 1): To a stirred solution of 1-4 (0.15 g, 0.49 mmol) in DMF (2.5 mL), DIPEA (0.26 mL, 1.47 mmol) was added followed by HATU (0.28 g, 0.73 mmol) at 0° C. and the reaction mixture was stirred at RT for 15 min. To the resulting reaction mixture, (3R)-3-methylmorpholine 1-5 (0.06 g, 0.58 mmol) was added and the reaction mixture was stirred at RT for 16 h. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The compound was purified by silica gel column chromatography (10% MeOH/DCM) to afford Example 1 (0.08 g). ¹H NMR (400 MHz, DMSO-d₆) δ=10.50 (s, 1H), 7.70-7.66 (m, 1H), 7.34-7.22 (m, 3H), 7.16-7.09 (m, 1H), 7.03-6.91 (m, 2H), 4.01-3.65 (m, 2H), 3.56 (d, 1H), 3.48-3.40 (m, 1H), 3.30-3.23 (m, 2H), 3.20-3.07 (m, 1H), 2.29 (s, 3H), 1.15 (d, 3H); LCMS: found [M+H]+=389.

### Example 2: Synthesis of N-(3-(2-cyanopiperidine-1-carbonyl)phenyl)-2,5-dimethylbenzenesulfonamide

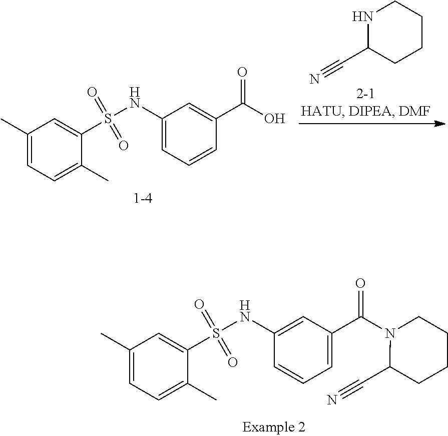 [SMILES: Cc1ccc(C)c(S(=O)(=O)Nc2cccc(C(=O)O)c2)c1 ; N#CC1CCCCN1 ; Cc1ccc(C)c(S(=O)(=O)Nc2cccc(C(=O)N3CCCCC3C#N)c2)c1]

To a stirred solution of 3-[(2,5-dimethylphenyl)sulfonylamino]benzoic acid 1-4 (0.15 g, 0.49 mmol) in DMF (2.5 mL), DIPEA (0.22 mL, 1.47 mmol) was added followed by HATU (0.28 g, 0.73 mmol) at 0° C. and the reaction mixture was stirred at RT for 15 min. To the resulting reaction mixture, piperidine-2-carbonitrile 2-1 (0.07 g, 0.65 mmol) was added and the reaction mixture was stirred at RT for 16 h. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The compound was purified by silica gel column chromatography (10% MeOH/DCM) to afford Example 2 (0.07 g). ¹H NMR (400 MHz, DMSO-d₆) δ=10.59 (s, 1H), 7.72-7.67 (m, 1H), 7.37-7.29 (m, 2H), 7.28-7.22 (m, 1H), 7.19-7.15 (m, 1H), 7.10-7.01 (m, 2H), 5.70-5.43 (m, 1H), 3.68-3.35 (m, 1H), 2.93 (t, 1H), 2.62-2.52 (m, 3H), 2.31-2.25 (m, 3H), 2.00-1.89 (m, 1H), 1.87-1.67 (m, 2H), 1.63-1.48 (m, 2H), 1.45-1.27 (m, 1H); LCMS: found [M+H]⁺=398.

### Example 3: Synthesis of 2,5-dimethyl-N-(3-(piperidine-1-carbonyl)phenyl)benzene sulfonamide

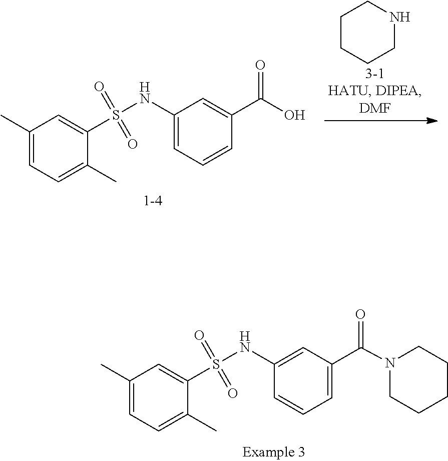 [SMILES: C1CCNCC1 ; Cc1ccc(C)c(S(=O)(=O)Nc2cccc(C(=O)O)c2)c1 ; Cc1ccc(C)c(S(=O)(=O)Nc2cccc(C(=O)N3CCCCC3)c2)c1]

To a stirred solution of 3-[(2,5-dimethylphenyl)sulfonylamino]benzoic acid 1-4 (0.15 g, 0.49 mmol) in DMF (2.5 mL), DIPEA (0.25 mL, 1.47 mmol) was added followed by HATU (0.28 g, 0.73 mmol) at 0° C. and the reaction mixture was stirred at RT for 15 min. To the resulting reaction mixture, piperidine 3-1 (0.05 g, 0.58 mmol) was added and the reaction mixture was stirred at RT for 16 h. After completion of reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The compound was purified by silica gel column chromatography (10% MeOH/DCM) to afford Example 3 (0.09 g). ¹H NMR (400 MHz, DMSO-d₆) δ=10.49 (s, 1H), 7.74-7.63 (m, 1H), 7.40-7.18 (m, 3H), 7.15-7.07 (m, 1H), 7.01-6.81 (m, 2H), 3.51 (br s, 2H), 3.05 (br s, 2H), 2.28 (s, 3H), 1.65-1.56 (m, 2H), 1.50 (br s, 2H), 1.33 (br s, 2H); LCMS: found [M+H]⁺=373.

### Example 4: Synthesis of N-(cyclopropylmethyl)-3-((2,5-dimethylphenyl)sulfonamido)benzamide

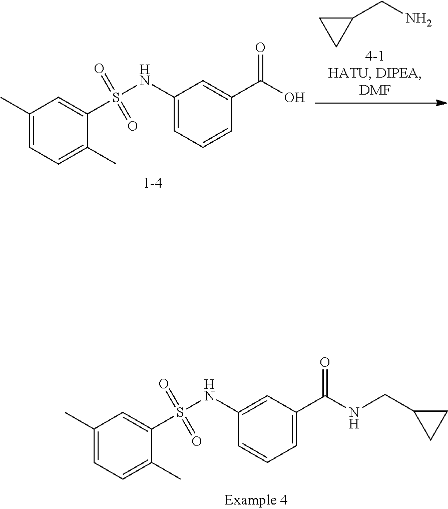 [SMILES: NCC1CC1 ; Cc1ccc(C)c(S(=O)(=O)Nc2cccc(C(=O)O)c2)c1 ; Cc1ccc(C)c(S(=O)(=O)Nc2cccc(C(=O)NCC3CC3)c2)c1]

To a stirred solution of 3-[(2,5-dimethylphenyl)sulfonylamino]benzoic acid 1-4 (0.15 g, 0.49 mmol) in DMF (2 mL), DIPEA (0.26 mL, 1.47 mmol) was added followed by HATU (0.28 g, 0.74 mmol) at 0° C. and the reaction mixture was stirred at RT for 15 min. To the resulting reaction mixture, cyclopropylmethanamine 4-1 (0.04 g, 0.49 mmol) was added and the reaction mixture was stirred at RT for 16 h. After completion of reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The compound was purified by prep-HPLC to afford Example 4 (0.09 g). ¹H NMR (400 MHz, DMSO-d₆) δ=10.46 (br s, 1H), 8.46 (t, 1H), 7.75-7.67 (m, 1H), 7.55 (t, 1H), 7.44 (d, 1H), 7.33-7.22 (m, 3H), 7.20-7.15 (m, 1H), 3.09 (t, 2H), 2.52 (s, 3H), 2.29 (s, 3H), 1.05-0.93 (m, 1H), 0.44-0.37 (m, 2H), 0.23-0.15 (m, 2H); LCMS: found [M+H]⁺=357.

### Example 5: Synthesis of (R)-2,5-dimethyl-N-(3-(2-methylpiperidine-1-carbonyl)phenyl)benzenesulfonamide

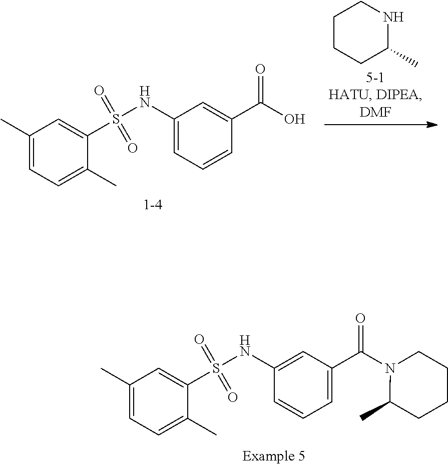

To a stirred solution of 3-[(2,5-dimethylphenyl)sulfonylamino]benzoic acid 1-4 (0.16 g, 0.51 mmol) in DMF (5 mL), DIPEA (0.21 mL, 1.56 mmol) was added followed by HATU (0.30 g, 0.77 mmol) at 0° C. and the reaction mixture was stirred at RT for 15 min. To the resulting reaction mixture, methylpiperidine 5-1 (0.06 g, 0.62 mmol) was added and the reaction mixture was stirred at RT for 16 h. After completion of the reaction, the reaction mixture was diluted with water, the aqueous layer was extracted with ethyl acetate. The organic layer was washed with saturated NaHCO₃ solution and brine, dried over anhydrous Na₂SO₄ and concentrated under reduced pressure. The compound was purified by silica gel column chromatography (10% MeOH/DCM) to afford Example 5 (0.03 g). ¹H NMR (400 MHz, DMSO-d₆) δ=10.46 (br s, 1H), 7.68 (s, 1H), 7.39-7.17 (m, 3H), 7.14-7.06 (m, 1H), 7.02-6.87 (m, 2H), 3.31 (s, 3H), 2.96-2.77 (m, 1H), 2.52 (br s, 3H), 2.28 (s, 3H), 1.69-1.41 (m, 4H), 1.37-1.18 (m, 1H), 1.10 (d, 3H); LCMS: found [M+H]⁺=387.

### Example 6: Synthesis of (S)-2,5-dimethyl-N-(3-(2-methylpiperidine-1-carbonyl)phenyl)benzenesulfonamide

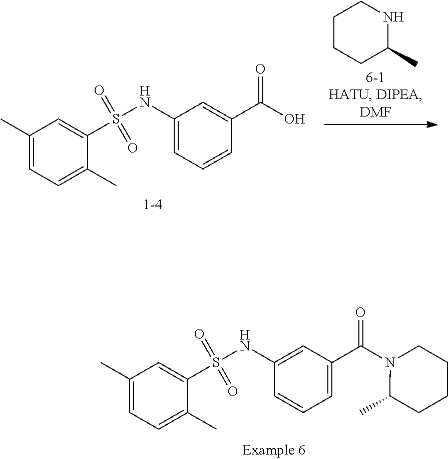

To a stirred solution of 3-[(2,5-dimethylphenyl)sulfonylamino]benzoic acid 1-4 (0.15 g, 0.49 mmol) in DMF (5 mL), DIPEA (0.20 mL, 1.47 mmol) was added followed by HATU (0.28 g, 0.74 mmol) at 0° C. and the reaction mixture was stirred at RT for 15 min. To the resulting reaction mixture, (2S)-2-methylpiperidine 6-1 (0.06 g, 0.59 mmol) was added and the reaction mixture was stirred at RT for 16 h. After completion of reaction, the reaction mixture was diluted with water, the aqueous layer was extracted with ethyl acetate. The organic layer was washed with saturated NaHCO₃ solution and brine, dried over anhydrous Na₂SO₄ and concentrated under reduced pressure. The compound was purified by silica gel column chromatography (10% MeOH/DCM) to afford Example 6 (0.07 g). ¹H NMR (400 MHz, DMSO-d₆) δ=10.48 (s, 1H), 7.68 (s, 1H), 7.34-7.22 (m, 3H), 7.15-7.06 (m, 1H), 6.98-6.91 (m, 2H), 3.31 (s, 3H), 2.86 (t, 1H), 2.52 (s, 3H), 2.28 (s, 3H), 1.67-1.41 (m, 4H), 1.25 (d, 1H), 1.10 (d, 3H); LCMS: found [M+H]⁺=387.

### Example 7: Synthesis of N-(3-(3-cyanomorpholine-4-carbonyl)phenyl)-2,5-dimethyl benzenesulfonamide

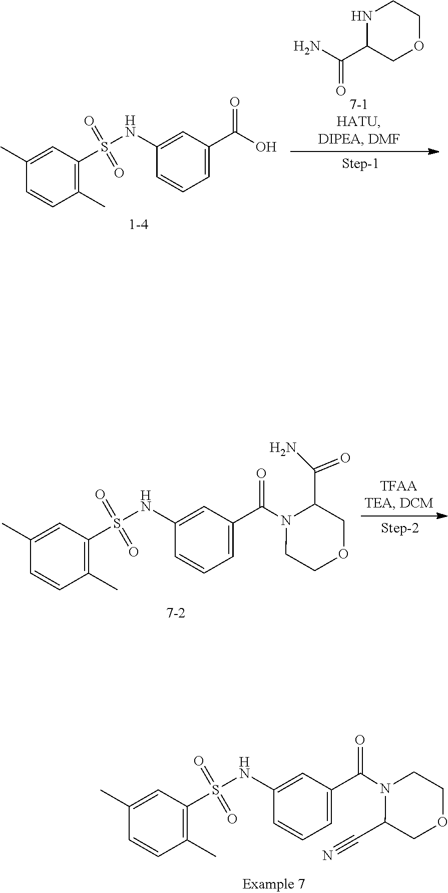 [SMILES: NC(=O)C1COCCN1 ; Cc1ccc(C)c(S(=O)(=O)Nc2cccc(C(=O)O)c2)c1 ; Cc1ccc(C)c(S(=O)(=O)Nc2cccc(C(=O)N3CCOCC3C(N)=O)c2)c1 ; Cc1ccc(C)c(S(=O)(=O)Nc2cccc(C(=O)N3CCOCC3C#N)c2)c1]

Step-1: 4-(3-((2,5-dimethylphenyl)sulfonamido)benzoyl)morpholine-3-carboxamide (7-2): To a stirred solution of 3-[(2,5-dimethylphenyl)sulfonylamino]benzoic acid 1-4 (0.24 g, 0.77 mmol) in DMF (2.5 mL), DIPEA (0.20 mL, 1.15 mmol) was added followed by HATU (0.35 g, 0.92 mmol) at 0° C. and the reaction mixture was stirred at RT for 15 min. To the resulting reaction mixture, morpholine-3-carboxamide 7-1 (0.10 g, 0.77 mmol) was added and the reaction mixture was stirred at RT for 16 h. After completion of the reaction, the reaction mixture was diluted with water. The aqueous layer was extracted with ethyl acetate. The organic layer was washed with ice cold water and brine, dried over anhydrous Na₂SO₄ and concentrated under reduced pressure. The compound was purified by combi flash (50% EtOAc/hexane) to afford 7-2 (0.07 g). ¹HNMR (400 MHz, DMSO-d₆) δ=10.52 (s, 1H), 7.72 (br s, 1H), 7.57-7.39 (m, 1H), 7.35-7.18 (m, 4H), 7.16-6.97 (m, 3H), 4.80-4.69 (m, 1H), 4.29 (d, 1H), 4.20-4.06 (m, 1H), 3.93-3.73 (m, 1H), 3.60 (br s, 1H), 3.40 (d, 1H), 2.98 (d, 1H), 2.29 (s, 3H); LCMS: found [M+H]⁺=418.

Step-2: N-(3-(3-cyanomorpholine-4-carbonyl)phenyl)-2,5-dimethylbenzenesulfonamide (Example 7): To a stirred solution of 7-2 (0.07 g, 0.17 mmol) in DCM (2.5 mL), TEA (0.09 mL, 0.67 mmol) was added at 0° C. and stirred for 15 min. To the resulting reaction mixture, TFAA (0.04 mL, 0.25 mmol) was added and stirred at RT for 1 h. After completion of the reaction, the reaction mixture was diluted with DCM. The organic layer was washed with water and brine, dried over anhydrous Na₂SO₄ and concentrated under reduced pressure. The compound was purified by prep HPLC to afford Example 7 (0.03 g). ¹H NMR (400 MHz, DMSO-d₆) δ=10.61 (br s, 1H), 7.71 (s, 1H), 7.37-7.29 (m, 2H), 7.27-7.23 (m, 1H), 7.21-7.17 (m, 1H), 7.10-7.04 (m, 2H), 5.45-5.27 (m, 1H), 4.07-3.97 (m, 1H), 3.80 (d, 1H), 3.65 (dd, 1H), 3.48-3.39 (m, 1H), 3.20-3.09 (m, 1H), 2.52 (s, 3H), 2.30 (s, 3H); LCMS: found [M+H]⁺=400.

### Example 8: Synthesis of (S)-N-(1-cyclopropylethyl)-3-((2,5-dimethylphenyl) sulfonamido)benzamide

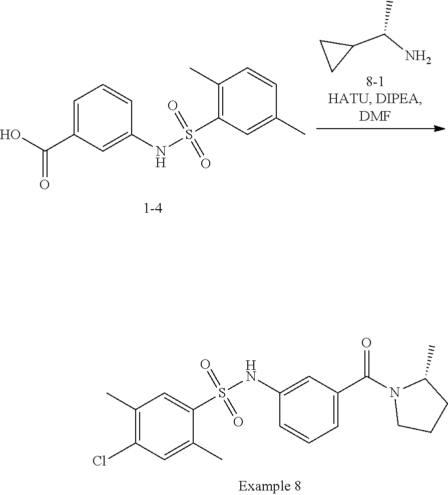

To a stirred solution of 3-[(2,5-dimethylphenyl)sulfonylamino]benzoic acid 1-4 (0.20 g, 0.66 mmol) in DMF (2.5 mL), DIPEA (0.34 mL, 1.96 mmol) was added followed by HATU (0.30 g, 0.79 mmol) at 0° C. and the reaction mixture was stirred at RT for 15 min. To the resulting reaction mixture, (S)-1-cyclopropylethan-1-amine.hydrochloride 8-1 (0.12 g, 0.98 mmol) was added and the reaction mixture was stirred at RT for 2 h. After completion of the reaction, the reaction mixture was diluted with water. The precipitated solid was filtered and washed with water. The compound was triturated with acetonitrile and n-pentane and dried under reduced pressure to afford Example 8 (0.13 g). ¹H NMR (400 MHz, DMSO-d₆) δ=9.27 (br s, 1H), 7.99 (s, 1H), 7.89 (d, 2H), 7.53 (t, 2H), 7.42-7.27 (m, 1H), 6.31 (d, 1H), 3.27-3.17 (m, 1H), 1.16 (d, 3H), 0.88 (br s, 1H), 0.41 (br s, 2H), 0.32-0.16 (m, 2H); LCMS: found [M+H]⁺=373.

### Example 9: Synthesis of (R)-4-chloro-2,5-dimethyl-N-(3-(2-methylpiperidine-1-carbonyl)phenyl)benzenesulfonamide

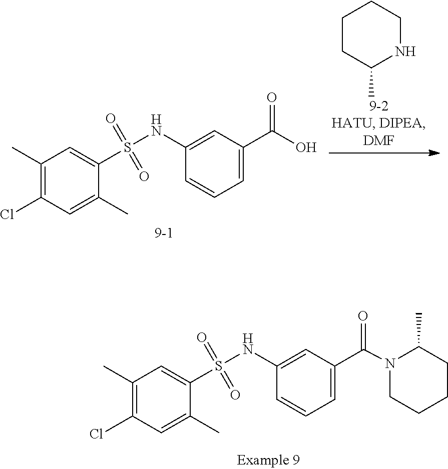

To a stirred solution of 9-1 (0.20 g, 0.59 mmol) in DMF (2.5 mL), DIPEA (0.31 mL, 1.77 mmol) was added followed by HATU (0.27 g, 0.71 mmol) at 0° C. and the reaction mixture was stirred at RT for 15 min. To the resulting reaction mixture, (2R)-2-methylpiperidine.hydrochloride 9-2 (0.12 g, 0.88 mmol) was added and the reaction mixture was stirred at RT for 2 h. After completion of the reaction, the reaction mixture was diluted with water. The precipitated solid was filtered, washed with water, n-pentane and dried under reduced pressure to afford Example 9 (0.19 g). ¹H NMR (400 MHz, DMSO-d₆) δ=7.53-7.47 (m, 1H), 7.37-7.24 (m, 1H), 7.11-7.02 (m, 1H), 4.71 (br s, 2H), 3.83 (br s, 2H), 3.22 (br s, 2H), 2.14 (s, 4H), 1.85-1.76 (m, 2H), 1.06 (d, 2H), 0.98-0.89 (m, 2H); LCMS: found [M+H]⁺=421.

### Example 10: Synthesis of (R)-4-chloro-2,5-dimethyl-N-(3-(2-methylpyrrolidine-1-carbonyl)phenyl)benzenesulfonamide

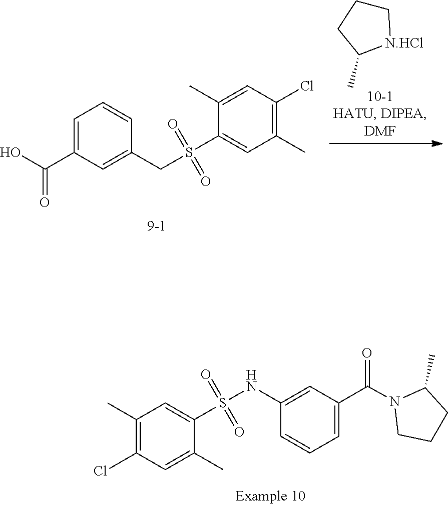

To a stirred solution of 3-((4-chloro-2,5-dimethylphenyl)sulfonamido)benzoic acid 9-1 (0.20 g, 0.59 mmol) in DMF (2.5 mL), DIPEA (0.31 mL, 1.77 mmol) was added followed by HATU (0.27 g, 0.71 mmol) at 0° C. and the reaction mixture was stirred at RT for 15 min. To the resulting reaction mixture, (2R)-2-methylpyrrolidine.hydrochloride 10-1 (0.11 g, 0.88 mmol) was added and the reaction mixture was stirred at RT for 2 h. After completion of the reaction, the reaction mixture was diluted with water. The precipitated solid was filtered, washed with water, n-pentane and dried under reduced pressure to afford Example 10 (0.02 g). ¹H NMR (400 MHz, DMSO-d₆) δ=10.25 (br s, 1H), 7.79 (br s, 1H), 7.43 (br s, 1H), 7.28 (d, 1H), 7.19-7.04 (m, 3H), 4.13-3.81 (m, 1H), 3.37-3.14 (m, 2H), 3.04 (br s, 3H), 2.53 (br s, 3H), 2.31 (br s, 3H), 2.02 (br s, 1H), 1.92-1.64 (m, 2H), 1.61-1.47 (m, 1H); LCMS: found [M+H]⁺=407.

### Example 11: Synthesis of N-benzyl-3-((4-chloro-2,5-dimethylphenyl)sulfonamido)benzamide

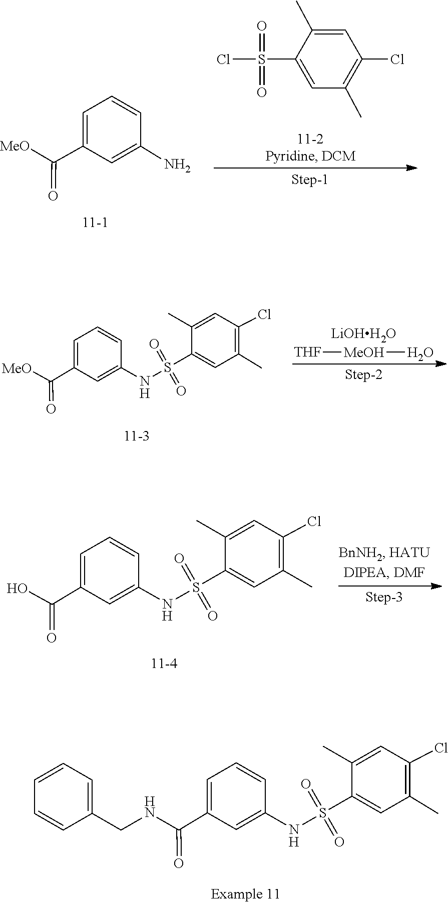

Step-1: methyl 3-((4-chloro-2,5-dimethylphenyl)sulfonamido)benzoate (11-3): To a stirred solution of methyl 3-aminobenzoate 11-1 (0.50 g, 3.31 mmol) in DCM (5 mL) was added pyridine (0.52 g, 6.62 mmol) at 0° C. followed by 4-chloro-2,5-dimethyl-benzenesulfonyl chloride 11-2 (0.79 g, 3.31 mmol) and the reaction mixture was stirred at RT for 2 h. After completion, the reaction mixture was quenched with water. The aqueous layer was extracted with 5% MeOH/DCM. The combined organic layer was dried over anhydrous Na₂SO₄ and concentrated under reduced pressure to afford compound as pale-yellow liquid. The product was purified by combiflash column (40% EtOAc/hexane) to afford 11-3 (0.80 g). ¹H NMR (400 MHz, DMSO-d₆) δ=10.70 (s, 1H), 7.87-7.85 (m, 1H), 7.65 (s, 1H), 7.58-7.54 (m, 1H), 7.46 (s, 1H), 7.39-7.30 (m, 2H), 3.82-3.79 (m, 3H), 2.51-2.49 (m, 3H), 2.31 (s, 3H).

Step-2: 3-((4-chloro-2,5-dimethylphenyl)sulfonamido)benzoic acid (11-4): To a stirred solution of 11-3 (0.60 g, 1.70 mmol) in THF (6 mL), methanol (3 mL) and water (2 mL) was added LiOH·H₂O (0.21 g, 5.09 mmol) at 0° C. and the reaction mixture was stirred at RT for 4 h. After completion, the reaction mixture was concentrated under reduced pressure and the residue was quenched with water. The aqueous layer was acidified with 6N HCl to pH-3 and extracted with 10% MeOH/DCM. The combined organic layer was dried over anhydrous Na₂SO₄ and concentrated under reduced pressure to afford 11-4 (0.40 g). ¹H NMR (400 MHz, DMSO-d₆) δ=7.83 (s, 1H), 7.63 (br s, 1H), 7.52 (br s, 1H), 7.45 (s, 1H), 7.27 (dd, 2H), 2.48-2.42 (m, 3H), 2.29 (s, 3H).

Step-3: N-benzyl-3-((4-chloro-2,5-dimethylphenyl)sulfonamido)benzamide (Example 11): To a stirred solution of 11-4 (0.10 g, 0.29 mmol) in DMF (2 mL) was added DIPEA (0.11 g, 0.88 mmol) and HATU (0.16 g, 0.41 mmol) at 0° C. followed by benzylamine (0.03 g, 0.29 mmol) and the reaction mixture was stirred at RT for 12 h. After completion, the reaction mixture was poured into ice cold water, the aqueous layer was extracted with DCM. The combined organic layer was washed with saturated NaHCO₃ solution and brine, dried over anhydrous Na₂SO₄ and concentrated under reduced pressure. The product was purified by prep HPLC to afford Example 11 (0.02 g, 16%) as a colorless oil. ¹H NMR (400 MHz, DMSO-d₆) δ=10.57 (s, 1H), 8.97-8.93 (m, 1H), 7.84 (s, 1H), 7.56 (s, 1H), 7.50 (d, 1H), 7.46-7.44 (m, 1H), 7.32-7.20 (m, 7H), 4.41 (d, 2H), 2.49-2.47 (m, 3H), 2.27 (s, 3H); LCMS: found [M+H]⁺=429.

### Example 12: Synthesis of 3-((4-chloro-2,5-dimethylphenyl)sulfonamido)-N,N-dimethylbenzamide

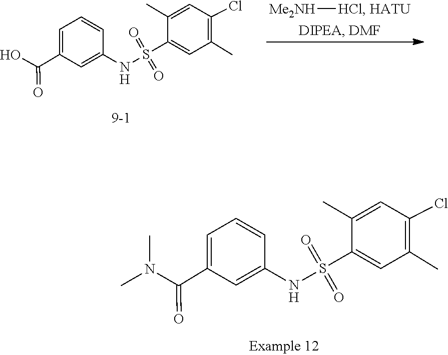

To a stirred solution of 9-1 (0.10 g, 0.29 mmol) in DMF (1.5 mL) was added DIPEA (0.16 mL, 0.88 mmol) and HATU (0.13 g, 0.35 mmol) at 0° C. followed by N,N-dimethylmethanamine hydrochloride (0.04 g, 0.44 mmol) and the reaction mixture was stirred at RT for 16 h. After completion, the reaction mixture was quenched with ice-cold water and the aqueous layer was extracted with ethyl acetate. The combined organic layer was washed with brine, dried over anhydrous Na₂SO₄ and concentrated under reduced pressure. The product was purified by prep HPLC to afford Example 12 (0.08 g). ¹H NMR (400 MHz, DMSO-d₆) δ=10.57-10.53 (m, 1H), 7.82 (s, 1H), 7.45 (s, 1H), 7.30-7.24 (m, 1H), 7.12-7.07 (m, 1H), 7.03-6.98 (m, 2H), 2.92 (br s, 3H), 2.70 (br s, 3H), 2.49-2.47 (m, 3H), 2.28 (s, 3H); LCMS: found [M+H]⁺=367.

### Example 13: Synthesis of 4-chloro-N-[3-fluoro-5-[(2R)-2-methylpyrrolidine-1-carbonyl]phenyl]-2,5-dimethyl-benzenesulfonamide

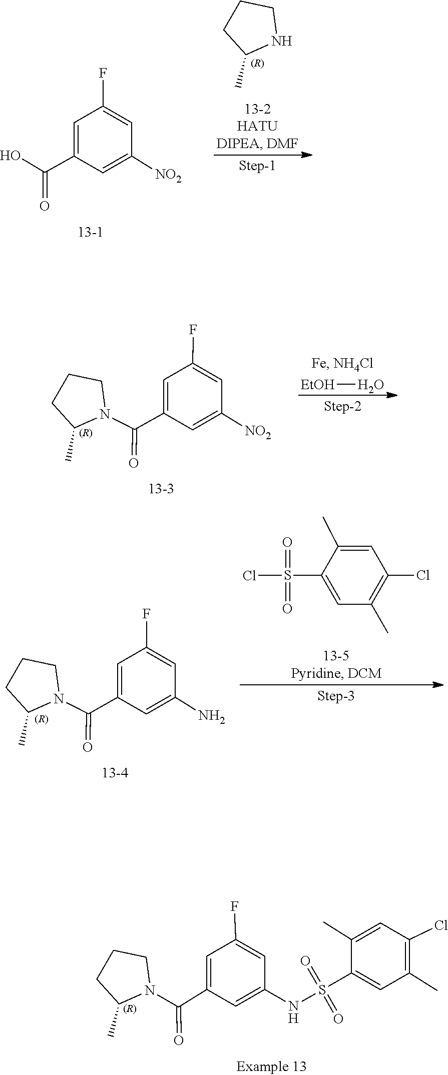

Step-1: (3-fluoro-5-nitro-phenyl)-[(2R)-2-methylpyrrolidin-1-yl]methanone (13-3): To a stirred solution of 13-1 (0.56 g, 3.00 mmol) in DMF (7.5 mL) was added DIPEA (1.2 mL, 9.00 mmol) and HATU (1.37 g, 3.60 mmol) at 0° C. followed by addition of (2R)-2-methylpyrrolidine 13-2 (0.38 g, 4.50 mmol) and the reaction mixture was stirred at RT for 3 h. After completion, the reaction was quenched with water and extracted with ethyl acetate. The organic layer was washed with brine, dried over anhydrous Na₂SO₄ and concentrated under reduced pressure. The product obtained was purified by combiflash column (30% EtOAc/n-heptane) to afford 13-3 (0.70 g). ¹H NMR (400 MHz, DMSO-d₆) δ=8.19 (d, 1H), 8.12 (s, 1H), 7.87 (d, 1H), 4.20-4.11 (m, 1H), 3.51 (d, 1H), 2.14-2.04 (m, 1H), 1.89 (dd, 1H), 1.77-1.68 (m, 1H), 1.62-1.54 (m, 1H), 1.27 (d, 3H), 0.88-0.82 (m, 1H); LCMS: found [M+H]⁺=253.

Step-2: (3-amino-5-fluoro-phenyl)-[(2R)-2-methylpyrrolidin-1-yl]methanone (13-4): To a stirred solution of 13-3 (0.38 g, 1.51 mmol) in ethanol (5 mL) and water (2.5 mL) was added iron powder (0.42 g, 7.53 mmol) followed by NH₄Cl (0.40 g, 7.53 mmol) and the reaction mixture was stirred at 80° C. for 2 h. After completion, the reaction mixture was concentrated under reduced pressure. The product was triturated with ethyl acetate and the suspension was filtered. The filtrate was concentrated under reduced pressure to afford 13-4 (0.38 g). ¹H NMR (400 MHz, DMSO-d₆) δ=6.47-6.28 (m, 3H), 5.56 (br s, 2H), 4.12-4.01 (m, 1H), 3.47-3.38 (m, 1H), 2.09-1.99 (m, 1H), 1.90-1.81 (m, 1H), 1.72-1.61 (m, 1H), 1.58-1.48 (m, 1H), 1.25-1.13 (m, 3H), 0.88 (d, 1H); LCMS: found [M+H]⁺=223.

Step-3: 4-chloro-N-[3-fluoro-5-[(2R)-2-methylpyrrolidine-1-carbonyl]phenyl]-2,5-dimethyl-benzenesulfonamide (Example 13): To a stirred solution of 13-4 (0.38 g, 1.71 mmol) in DCM (3 mL) was added pyridine (0.41 mL, 5.13 mmol) at 0° C. followed by 4-chloro-2,5-dimethyl-benzenesulfonyl chloride 13-5 (0.41 g, 1.71 mmol). The reaction mixture was stirred at RT for 2 h. After completion, the reaction mixture was quenched with water. The aqueous layer was extracted with DCM. The combined organic layer was washed with brine, dried over anhydrous Na₂SO₄ and concentrated under reduced pressure. Product was purified by combi-flash column (25% EtOAc/n-heptane) to afford Example 13 (0.10 g). ¹H NMR (VT NMR) (400 MHz, DMSO-d₆) δ=10.55 (br s, 1H), 7.84 (s, 1H), 7.46 (s, 1H), 6.96-6.90 (m, 3H), 4.10-3.92 (m, 1H), 3.27 (br s, 2H), 2.54 (s, 3H), 2.33 (s, 3H), 2.09-2.01 (m, 1H), 1.85 (dd, 1H), 1.73 (br s, 1H), 1.54 (dd, 1H), 1.21-0.91 (m, 3H); LCMS: found [M+H]⁺=425.

### Example 14-15: Synthesis of chiral analogs of 3-[(4-chloro-2,5-dimethyl-phenyl)sulfonylamino]-N-[2-hydroxy-1-methyl-ethyl]-N-methyl-benzamide

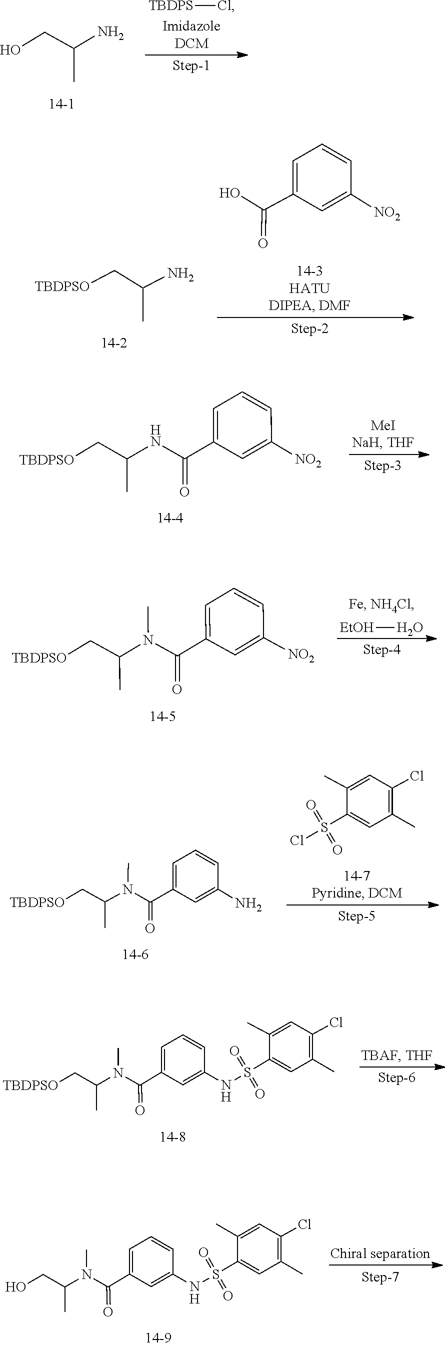

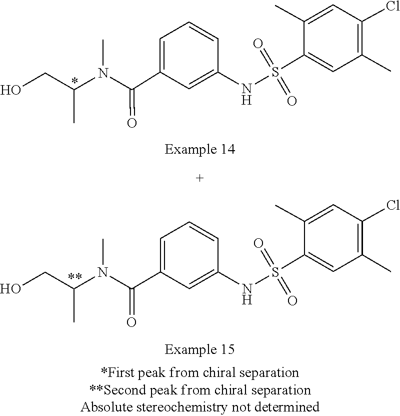 [SMILES: Cc1cc(S(=O)(=O)Nc2cccc(C(=O)N(C)C(C)CO)c2)c(C)cc1Cl ; Cc1cc(S(=O)(=O)Nc2cccc(C(=O)N(C)C(C)CO)c2)c(C)cc1Cl]

Step-1: 1-[tert-butyl(diphenyl)silyl]oxypropan-2-amine (14-2): To a stirred solution of 2-aminopropan-1-ol 14-1 (0.70 g, 9.32 mmol) in DCM (20 mL) was added imidazole (1.20 g, 18.60 mmol) at 0° C. followed by TBDPS-Cl (2.60 g, 9.79 mmol) and the reaction mixture was stirred at 40° C. for 3 h. After completion, the reaction mixture was quenched with ice cold water and extracted with DCM. The combined organic layers were washed with brine, dried over anhydrous Na₂SO₄ and evaporated under reduced pressure to obtain 14-2 (2.70 g) which was used as such for the next reaction. LCMS: found [M+H]⁺=314.

Step-2: N-[2-[tert-butyl(diphenyl)silyl]oxy-1-methyl-ethyl]-3-nitro-benzamide (14-4): To a stirred solution of 3-nitrobenzoic acid 14-3 (1.20 g, 7.18 mmol) in DMF (10 mL) was added DIPEA 2.78 g, 21.5 mmol) and HATU (3.28 g, 8.62 mmol) at 0° C. followed by 14-2 (2.93 g, 9.33 mmol). The reaction mixture was stirred at RT for 16 h. After completion, the reaction mixture was quenched with ice cold water and extracted with ethyl acetate. The organic layer was washed with brine, dried over anhydrous Na₂SO₄ and concentrated under reduced pressure. The product obtained was purified by combiflash (30% EtOAc/Hexane) to afford 14-4 (2.70 g). ¹H NMR (400 MHz, DMSO-d₆) δ=8.71-8.67 (m, 1H), 8.62 (d, 1H), 8.37 (d, 1H), 8.28 (d, 1H), 7.77 (t, 1H), 7.62 (br s, 4H), 7.47-7.37 (m, 6H), 4.30-4.23 (m, 1H), 3.76-3.69 (m, 1H), 3.68-3.62 (m, 1H), 1.23 (d, 3H), 0.98 (s, 9H); LCMS: found [M+H]⁺=463.

Step-3: N-[2-[tert-butyl(diphenyl)silyl]oxy-1-methyl-ethyl]-N-methyl-3-nitro-benzamide (14-5): To a stirred solution of 14-4 (0.50 g, 1.08 mmol) in THF (20 mL) was added NaH (60% in mineral oil, 0.13 g, 5.40 mmol) at 0° C. and the reaction mixture was stirred for 20 min. To the resulting reaction mixture was added methyl iodide (0.34 mL, 5.40 mmol) and stirred at 0° C. for 30 min, followed by 70° C. for 5 h. After completion, the reaction mixture was quenched with water and extracted with ethyl acetate. The organic layer was washed with brine, dried over anhydrous Na₂SO₄ and concentrated under reduced pressure. The product obtained was purified by combiflash column (30% EtOAc/n-hexanes) to afford 14-5 (0.25 g). LCMS: found [M+H]⁺=478.

Step-4: 3-amino-N-[2-[tert-butyl(diphenyl)silyl]oxy-1-methyl-ethyl]-N-methyl-benzamide (14-6): To a stirred solution of 14-5 (0.20 g, 0.42 mmol) in ethanol (15 mL) and water (1 mL), ammonium chloride (0.11 g, 2.09 mmol) and iron powder (0.11 g, 2.09 mmol) were added and the reaction mixture was stirred at 90° C. for 5 h. After completion, reaction mixture was filtered through celite and washed with EtOH. The filtrate was concentrated under reduced pressure. The product was suspended in ethyl acetate and stirred. It was filtered and the filtrate was concentrated under reduced pressure to afford 14-6 (0.14 g). LCMS [M+H]⁺=447.

Step-5: N-[2-[tert-butyl(diphenyl)silyl]oxy-1-methyl-ethyl]-3-[(4-chloro-2,5-dimethyl-phenyl)sulfonylamino]-N-methyl-benzamide (14-8): To a solution of 14-6 (0.14 g, 0.31 mmol) in DCM (4 mL) was added pyridine (0.05 mL, 0.62 mmol) and the reaction mixture was stirred at 0° C. for 15 min. To the resulting reaction mixture was added 4-chloro-2,5-dimethyl-benzenesulfonyl chloride 14-7 (0.07 g, 0.31 mmol) and was stirred at RT for 2 h. After completion, the reaction mixture was quenched with water and extracted with ethyl acetate. The organic layer was washed with brine, dried over anhydrous Na₂SO₄ and concentrated under reduced pressure. The product obtained was purified by combiflash (30% EtOAc/Hexanes) to afford 14-8 (0.10 g). LCMS: found [M+H]⁺=650.

Step-6: Chiral analogs of 3-[(4-chloro-2,5-dimethyl-phenyl)sulfonylamino]-N-[2-hydroxy-1-methyl-ethyl]-N-methyl-benzamide (Examples 14 and 15): To a stirred solution of 14-8 (1.00 g, 1.54 mmol) in THF (20 mL) was added TBAF (0.81 g, 3.08 mmol) at 0° C. and the reaction mixture was stirred at RT for 16 h. After completion, the reaction mixture was diluted with water and extracted with ethyl acetate. The organic layer was washed with brine, dried over anhydrous Na₂SO₄ and concentrated under reduced pressure. The product obtained was triturated with diethyl ether. Chiral prep HPLC purification of the product afforded Example 14 (0.05 g) and Example 15 (0.04 g). The absolute stereochemistry was not determined.

Example 14: ¹H NMR (400 MHz, DMSO-d₆) δ=10.60-10.39 (m, 1H), 7.86-7.81 (m, 1H), 7.47 (s, 1H), 7.29-7.23 (m, 1H), 7.10-7.07 (m, 1H), 7.02-6.97 (m, 2H), 4.77 (t, 1H), 4.55-4.49 (m, 1H), 3.54-3.38 (m, 2H), 3.18 (dd, 1H), 2.78-2.76 (m, 2H), 2.54 (br s, 3H), 2.31-2.30 (m, 3H), 0.91 (d, 3H); LCMS: found [M+H]⁺=411.

Example 15: ¹H NMR (400 MHz, DMSO-d₆) δ=10.55 (br s, 1H), 7.86-7.82 (m, 1H), 7.47 (s, 1H), 7.30-7.25 (m, 1H), 7.10-7.07 (m, 1H), 7.03-6.98 (m, 2H), 4.79-4.76 (m, 1H), 3.54-3.47 (m, 1H), 3.46-3.39 (m, 1H), 3.21-3.15 (m, 1H), 2.76 (s, 3H), 2.53-2.51 (m, 3H), 2.30 (s, 3H), 1.07-0.91 (m, 3H); LCMS: found [M+H]⁺=411.

### Example 16: Synthesis of4-chloro-2,5-dimethyl-N-(3-(thiazol-2-yl)phenyl)benzene sulfonamide

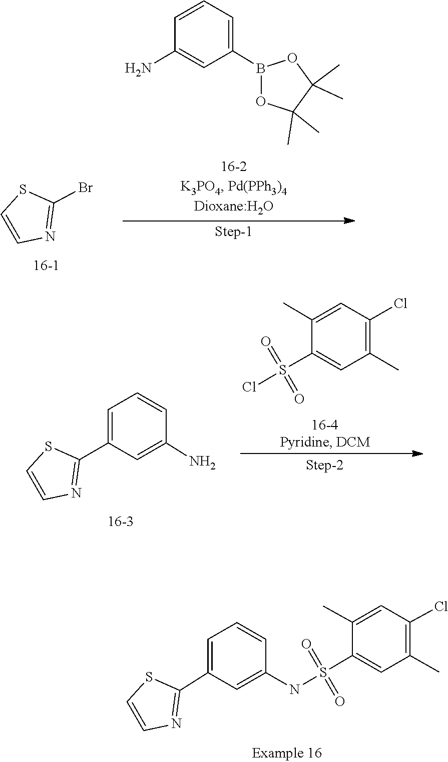

Step-1: 3-(thiazol-2-yl)aniline (16-3): To a stirred solution of 3-(4,4,5,5-tetramethyl-1,3,2-dioxaborolan-2-yl)aniline 16-2 (0.61 g, 2.77 mmol) and 2-bromothiazole 16-1 (0.35 g, 2.13 mmol) in 1,4-dioxane (25 mL) was added potassium phosphate (1.36 g, 6.40 mmol) dissolved in water (2 mL) and the reaction mixture was purged with argon for 30 min. To the resulting reaction mixture, tetrakis(triphenylphosphine)palladium(0) (0.12 g, 0.11 mmol) was added and the reaction mixture was again purged with argon for 20 min. The reaction mixture was stirred at 100° C. for 16 h. After completion of the reaction, the reaction mixture was filtered through celite and washed with ethyl acetate. The filtrate was diluted with cold water and extracted with ethyl acetate. Combined organic layer was washed with water and brine, dried over anhydrous Na₂SO₄ and concentrated under reduced pressure. The compound was purified by silica gel column chromatography (15% EtOAc/hexane) to afford 16-3 (0.17 g). LCMS: found [M+H]⁺=177.

Step-2: 4-chloro-2,5-dimethyl-N-(3-(thiazol-2-yl)phenyl)benzenesulfonamide (Example 16): To a stirred solution of 3-(thiazol-2-yl)aniline 16-3 (0.17 g, 0.97 mmol) in DCM (2.5 mL) was added pyridine (0.15 g, 1.93 mmol) followed by 4-chloro-2,5-dimethyl-benzenesulfonyl chloride 16-4 (0.23 g, 0.97 mmol) at 0° C. and the reaction was stirred at RT for 16 h. After completion, the reaction mixture was quenched with water and extracted with ethyl acetate. The organic layer was washed with brine, dried over anhydrous Na₂SO₄ and concentrated under reduced pressure. The product obtained was purified by combiflash (30% EtOAc/hexane) to afford Example 16 (0.10 g). ¹H NMR (400 MHz, DMSO-d₆) δ=10.73 (s, 1H), 7.92 (d, 2H), 7.79 (d, 1H), 7.69-7.68 (m, 1H), 7.57-7.54 (m, 1H), 7.48 (s, 1H), 7.38-7.33 (m, 1H), 7.19-7.12 (m, 1H), 2.54 (s, 3H), 2.32 (s, 3H); LCMS: found [M+H]⁺=379.

### Example 17: Synthesis of 4-chloro-2,5-dimethyl-N-(3-oxazol-2-ylphenyl)benzene sulfonamide

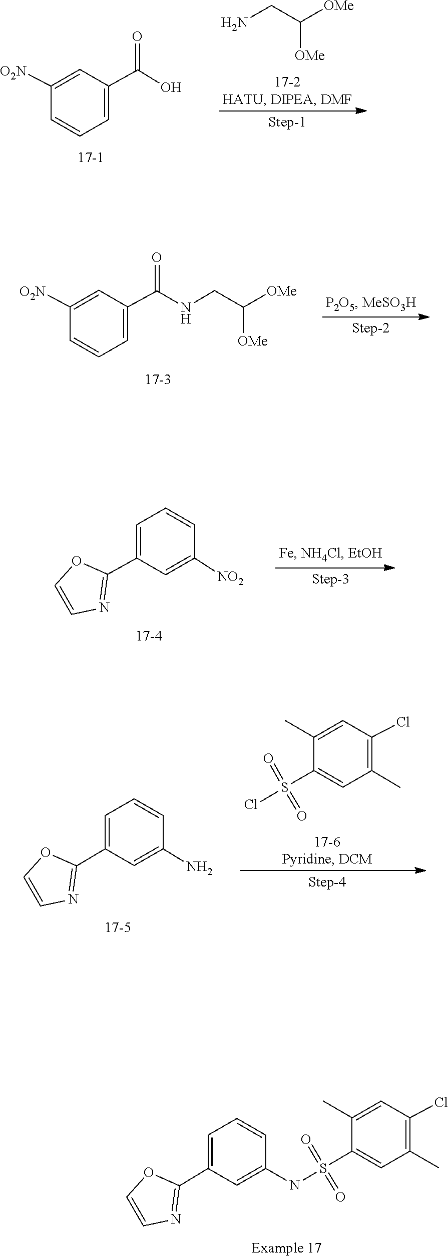

Step-1: N-(2-methoxypropyl)-3-nitro-benzamide (17-3): To a stirred solution of 3-nitrobenzoic acid 17-1 (1.00 g, 5.98 mmol) in DMF (5 mL) was added DIPEA (2.60 mL, 15.0 mmol) and HATU (2.73 g, 7.18 mmol) at 0° C. followed by 2,2-dimethoxyethanamine 17-2 (0.75 g, 7.18 mmol) and the reaction mixture was stirred at RT for 2 h. After completion, the reaction mixture was quenched with ice cold water, extracted with ethyl acetate. The organic layer was washed with brine, dried over anhydrous Na₂SO₄ and concentrated under reduced pressure. The product obtained was purified by combiflash (70% EtOAc/Heptane) to afford 17-3 (1.50 g) that was used directly in the next step.

Step-2: 2-(3-nitrophenyl)oxazole (17-4): To a stirred solution of 17-3 (1.00 g, 3.93 mmol) in methane sulphonic acid (2.50 mL, 39.30 mmol) was added phosphorous pentoxide (2.10 mL, 35.40 mmol) at 0° C. and the reaction mixture was stirred at 130° C. for 5 h. After completion, reaction mixture was quenched with saturated NaHCO₃, aqueous layer was extracted with ethyl acetate. Organic layer was washed with brine, dried over anhydrous Na₂SO₄, and concentrated under reduced pressure. Product obtained was purified by combi flash column (30% EtOAc/Hexane) to afford 17-4 (0.80 g). LCMS: found [M+H]⁺=191.

Step-3: 3-oxazol-2-ylaniline (17-5): To a stirred solution of 17-4 (0.20 g, 1.05 mmol) in ethanol (3 mL) was added iron powder (0.29 g, 5.26 mmol) followed by ammonium chloride (0.28 g, 5.26 mmol) dissolved in water (3 mL) and the reaction mixture was stirred at 80° C. for 2 h. After completion, the reaction mixture was filtered through Celite. The filtrate was concentrated under reduce pressure. The residue was diluted with water and extracted with ethyl acetate. The organic layer was dried over anhydrous Na₂SO₄ and concentrated under reduced pressure. The product obtained was purified by combiflash (60% EtOAc/Heptane) to afford 17-5 (0.12 g). LCMS: found [M+H]⁺=162.

Step-4: 4-chloro-2,5-dimethyl-N-(3-oxazol-2-ylphenyl)benzenesulfonamide (Example 17): To a stirred solution of 17-5 (0.10 g, 0.62 mmol) in DCM (3 mL) was added pyridine (0.14 mL, 1.25 mmol) at 0° C. followed by 4-chloro-2,5-dimethyl-benzenesulfonyl chloride 17-6 (0.14 g, 0.62 mmol) and the reaction mixture was stirred at RT for 16 h. After completion, the reaction mixture was quenched with water and extracted with DCM. The organic layer was washed with brine, dried over anhydrous Na₂SO₄ and concentrated under reduced pressure. The product obtained was purified by combiflash (50% EtOA/Hexane) to afford Example 17 (0.11 g, 49%) as a white solid. ¹H NMR (400 MHz, DMSO-d₆) δ=10.73 (br s, 1H), 8.21 (s, 1H), 7.89 (s, 1H), 7.72 (br s, 1H), 7.60 (d, 1H), 7.50-7.46 (m, 1H), 7.42-7.35 (m, 2H), 7.23-7.18 (m, 1H), 2.53 (s, 3H), 2.33-2.30 (m, 3H); LCMS: found [M+H]⁺=363.

### Example 18: Synthesis of 4-chloro-2,5-dimethyl-N-[3-(1-methylpyrazol-3-yl)phenyl]benzenesulfonamide

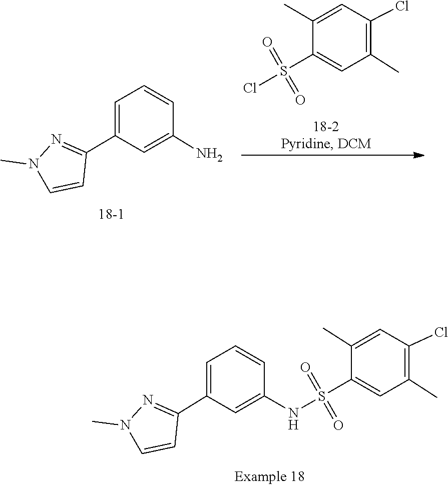 [SMILES: Cn1ccc(-c2cccc(N)c2)n1 ; Cc1cc(S(=O)(=O)Cl)c(C)cc1Cl ; Cc1cc(S(=O)(=O)Nc2cccc(-c3ccn(C)n3)c2)c(C)cc1Cl]

To a stirred solution of 18-1 (0.20 g, 1.15 mmol) in DCM (5 mL), was added pyridine (0.25 mL, 2.31 mmol) at 0° C. followed by 4-chloro-2,5-dimethyl-benzenesulfonyl chloride 18-2 (0.27 g, 1.15 mmol) and the reaction mixture was stirred at RT for 16 h. After completion, the reaction mixture was quenched with water, extracted with DCM. The organic layer was washed with brine, dried over anhydrous Na₂SO₄, and concentrated under reduced pressure. The product obtained was purified by combiflash (60% EtOAc/Hexane) to afford Example 18 (0.19 g). ¹H NMR (400 MHz, DMSO-d₆) δ=10.48-10.43 (m, 1H), 7.91-7.89 (m, 1H), 7.72-7.70 (m, 1H), 7.52-7.49 (m, 1H), 7.46 (s, 1H), 7.39-7.35 (m, 1H), 7.25-7.19 (m, 1H), 6.99-6.95 (m, 1H), 6.55-6.52 (m, 1H), 3.86 (s, 3H), 2.52 (s, 3H), 2.32 (s, 3H); LCMS: found [M+H]⁺=377.

### Example 19: Synthesis of 4-chloro-2,5-dimethyl-N-(3-(1-methyl-1H-imidazol-2-yl)phenyl)benzenesulfonamide

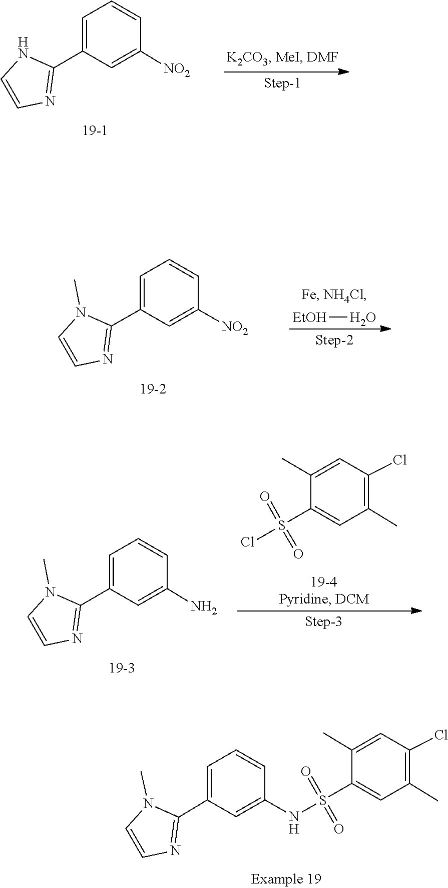

Step-1: 1-methyl-2-(3-nitrophenyl)-1H-imidazole (19-2): To a stirred solution of 19-1 (0.50 g, 2.64 mmol) in DMF (5 mL) was added potassium carbonate (0.91 g, 6.61 mmol) at 0° C. followed by iodomethane (0.33 mL, 5.29 mmol) and the reaction was stirred at 80° C. for 8 h. After completion, the reaction mixture was quenched with water and extracted with ethyl acetate. The combined organic layers were washed with brine, dried over anhydrous Na₂SO₄ and evaporated under reduced pressure. The product obtained was purified by combiflash (80% EtOAc/hexane) to afford 19-2 (0.40 g). LCMS: found [M+H]⁺=204.

Step-2: 3-(1-methyl-1H-imidazol-2-yl)aniline (19-3): To a stirred solution of 1-methyl-2-(3-nitrophenyl)-1H-imidazole 19-2 (0.30 g, 1.48 mmol) in ethanol (5 mL) was added ammonium chloride (0.40 g, 7.38 mmol) dissolved in water (2 mL) and iron powder (0.33 g, 5.91 mmol) at RT and the reaction mixture was stirred at 80° C. for 2 h. After completion, the reaction mixture was filtered through celite. The filtrate was concentrated, the residue was diluted with cold water and extracted with ethyl acetate. The combined organic layer was washed with water and brine, dried over anhydrous Na₂SO₄ and concentrated under reduced pressure to afford 19-3 (0.12 g). LCMS: found [M+H]⁺=174.

Step-3: 4-chloro-2,5-dimethyl-N-(3-(1-methyl-1H-imidazol-2-yl)phenyl)benzenesulfonamide (Example 19): To a stirred solution of 19-3 (0.10 g, 0.58 mmol) in DCM (4 mL) was added pyridine (0.13 mL, 1.15 mmol) at 0° C. followed by 4-chloro-2,5-dimethyl-benzenesulfonyl chloride 19-4 (0.14 g, 0.58 mmol) and the reaction mixture was stirred at RT for 6 h. After completion, the reaction mixture was quenched with water and aqueous layer was extracted with DCM. The organic layer was washed with brine, dried over anhydrous Na₂SO₄ and concentrated under reduced pressure. The product obtained was purified by combiflash (60% EtOAc/hexane) to afford Example 19 (0.05 g). ¹H NMR (400 MHz, DMSO-d₆) δ=10.99-10.95 (m, 1H), 7.96-7.95 (m, 1H), 7.77-7.75 (m, 1H), 7.71-7.69 (m, 1H), 7.53-7.49 (m, 2H), 7.47-7.44 (m, 1H), 7.42-7.39 (m, 1H), 7.35-7.32 (m, 1H), 3.73-3.71 (m, 3H), 2.58-2.55 (m, 3H), 2.33-2.32 (m, 3H); LCMS: found [M+H]⁺=376.

### Example 20: Synthesis of 4-chloro-2,5-dimethyl-N-(3-(4-methylthiazol-2-yl)phenyl)benzenesulfonamide

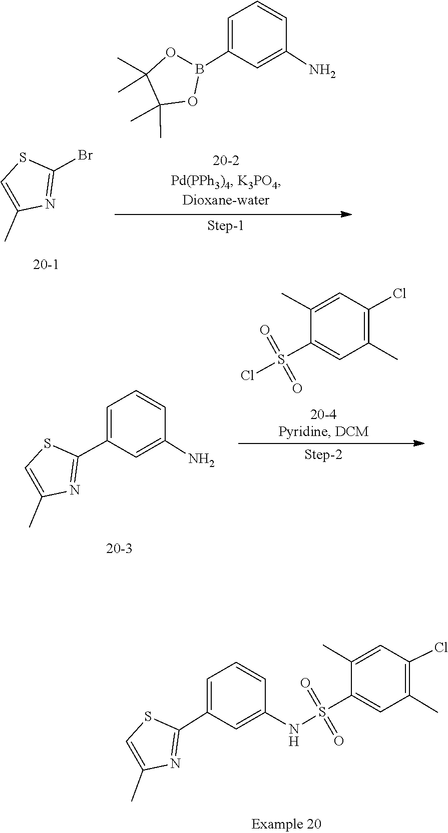

Step 1: 3-(4-methylthiazol-2-yl)aniline (20-3): To a stirred solution of 2-bromo-4-methyl-thiazole 20-1 (0.50 g, 2.81 mmol) in 1,4-dioxane (8 mL) and water (2 mL) was added in 3-(4,4,5,5-tetramethyl-1,3,2-dioxaborolan-2-yl)aniline 20-2 (0.74 g, 3.37 mmol) and K₃PO₄ (1.79 g, 8.42 mmol) and the reaction mixture was purged with argon for 30 min. To the resulting reaction mixture, tetrakis(triphenylphosphine)palladium(0) (0.65 g, 0.56 mmol) was added and the reaction mixture was again purged with argon for 10 min. The reaction mixture was stirred at 100° C. for 16 h. After completion of reaction, the reaction mixture was filtered through celite and washed with ethyl acetate. The filtrate was diluted with cold water and extracted with ethyl acetate. Combined organic layer was washed with water and brine, dried over anhydrous Na₂SO₄ and concentrated under reduced pressure. The crude compound was purified by combi flash column chromatography (10% EtOAc/hexane) to afford 20-3 (0.38 g). ¹H NMR (400 MHz, DMSO-d₆) δ=7.22 (d, 1H), 7.17 (t, 1H), 7.12-7.06 (m, 1H), 7.04-6.99 (m, 1H), 6.66-6.60 (m, 1H), 5.32 (s, 2H), 2.39 (s, 3H); LCMS: found [M+H]⁺=192.

Step-2: 4-chloro-2,5-dimethyl-N-(3-(4-methylthiazol-2-yl)phenyl)benzenesulfonamide (Example 20): To stirred a solution of 20-3 (0.20 g, 1.05 mmol) in DCM (10 mL) was added pyridine (0.25 mL, 3.15 mmol) followed by 4-chloro-2,5-dimethyl-benzenesulfonyl chloride 20-4 (0.25 g, 1.05 mmol) at 0° C. and the reaction was stirred at RT for 2 h. After completion, the reaction mixture was quenched with water and extracted with DCM. The organic layer was washed with brine, dried over anhydrous Na₂SO₄ and concentrated under reduced pressure. The crude obtained was purified by prep HPLC to afford Example 20 (0.10 g). ¹H NMR (400 MHz, DMSO-d₆) δ=10.69 (s, 1H), 7.93 (s, 1H), 7.64 (t, 1H), 7.52-7.48 (m, 2H), 7.35-7.31 (m, 2H), 7.14 (dd, 1H), 2.53 (s, 3H), 2.41 (d, 3H), 2.33 (s, 3H); LCMS: found [M+H]⁺=393.

### Example 21: Synthesis of 4-chloro-2,5-dimethyl-N-(3-(5-methylthiazol-2-yl)phenyl)benzenesulfonamide

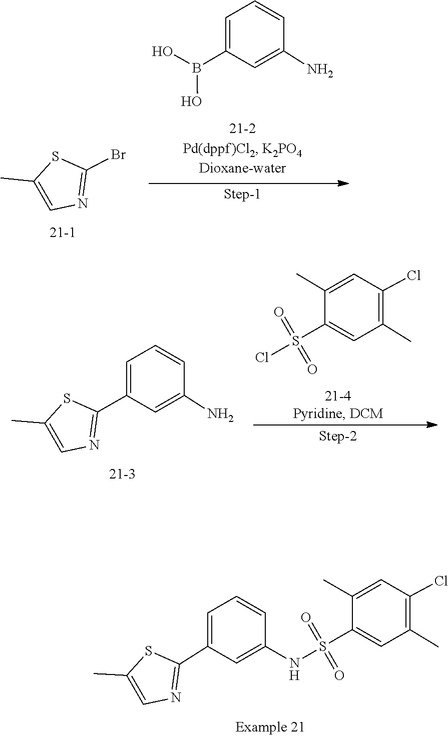

Step-1: 3-(5-methylthiazol-2-yl)aniline (21-3): To a stirred solution of 2-bromo-5-methyl-thiazole 21-1 (0.50 g, 2.81 mmol) in 1,4-dioxane (5 mL) and water (2 mL) was added (3-aminophenyl)boronic acid 21-2 (0.42 g, 3.09 mmol) and K₃PO₄ (1.79 g, 8.42 mmol) and the reaction mixture was purged with argon for 30 min. To the resulting reaction mixture, (1,1′-bis(diphenylphosphino)ferrocene)palladium(II) dichloride (0.04 g, 0.06 mmol) was added and the reaction mixture was again purged with argon for 10 min. The reaction mixture was stirred at 100° C. for 2 h. After completion of reaction, the reaction mixture was filtered through celite and washed with ethyl acetate. The filtrate was diluted with cold water and extracted with ethyl acetate. The combined organic layer was washed with water and brine, dried over anhydrous Na₂SO₄ and concentrated under reduced pressure. The crude compound was purified by combi flash column chromatography (40% EtOAc/hexane) to afford 21-3 (0.05 g). ¹H NMR (400 MHz, DMSO-d₆) δ=7.53 (s, 1H), 7.11-7.07 (m, 2H), 6.98 (d, 1H), 6.62 (dd, 1H), 5.32-5.28 (m, 2H), 2.46 (d, 3H); LCMS: found [M+H]⁺=192.

Step-2: 4-chloro-2,5-dimethyl-N-(3-(5-methylthiazol-2-yl)phenyl)benzenesulfonamide (Example 21): To a stirred solution of 21-3 (0.09 g, 0.47 mmol) in DCM (3 mL) was added pyridine (0.09 g, 1.18 mmol) followed by 4-chloro-2,5-dimethyl-benzenesulfonyl chloride 21-4 (0.14 g, 0.57 mmol) at 0° C. and the reaction was stirred at RT for 4 h. After completion, the reaction mixture was quenched with water and extracted with DCM. The organic layer was washed with brine, dried over anhydrous Na₂SO₄ and concentrated under reduced pressure. The crude obtained was purified by combi flash (40% EtOAc/hexane) to afford Example 21 (0.04 g). ¹H NMR (400 MHz, DMSO-d₆) δ=10.70 (s, 1H), 7.91 (s, 1H), 7.60 (d, 2H), 7.48 (s, 2H), 7.32 (t, 1H), 7.13 (dd, 1H), 2.53 (s, 3H), 2.48 (s, 3H), 2.32 (s, 3H); LCMS: found [M+H]⁺=393.

### Example 22: Synthesis of 4-chloro-N,2,5-trimethyl-N-(3-(thiazol-2-yl)phenyl)benzenesulfonamide

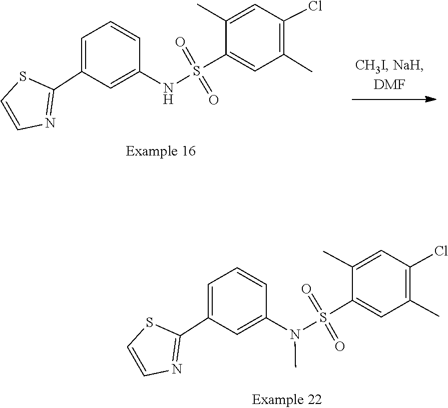 [SMILES: Cc1cc(S(=O)(=O)Nc2cccc(-c3nccs3)c2)c(C)cc1Cl ; Cc1cc(S(=O)(=O)N(C)c2cccc(-c3nccs3)c2)c(C)cc1Cl]

To stirred a solution of Example 16 (0.10 g, 0.26 mmol) in THF (5 mL) was added NaH (60% in mineral oil, 0.03 g, 1.31 mmol) at 0° C. and the reaction mixture was stirred for 30 min. To the resulting reaction mixture was added Mel (0.19 g, 1.31 mmol) at 0° C. and the reaction mixture was stirred at RT for 12 h. After completion, the reaction was quenched with water and extracted with ethyl acetate. The organic layer was washed with brine, dried over anhydrous Na₂SO₄ and concentrated under reduced pressure. The crude obtained was purified by combi flash (30% EtOAc/hexane) to afford Example 22 (0.03 g). ¹H NMR (400 MHz, DMSO-d₆) δ=7.94 (d, 1H), 7.88-7.85 (m, 1H), 7.83 (d, 1H), 7.74-7.72 (m, 2H), 7.51-7.47 (m, 2H), 7.32-7.30 (m, 1H), 3.23 (s, 3H), 2.33 (s, 3H), 2.15 (s, 3H); LCMS: found [M+H]⁺=393.

### Biological/Biochemical Evaluation

Exonuclease inhibition assay with human POLγ.

The ability of small molecules to inhibit the 3′-5′ exonuclease activity of POLγ was analyzed in a quantitative fluorescence SYBR Green I assay with fluorescence intensity (FI) readout.

Human POLγ was incubated with a linear, partially double-stranded template containing an 85 base pair double-stranded DNA stretch and a 15 nucleotides long single-stranded 5′-tail (hereafter referred to as the “exonuclease template”). The exonuclease template showed high fluorescence intensity when incubated with the DNA intercalator SYBR Green I. SYBR Green I is a double-stranded DNA-binding dye, which can be used to quantify double-stranded DNA. When bound to double-stranded DNA, SYBR Green I fluorescence increases by up to 100-fold. In the absence of deoxynucleotide triphosphates (dNTPs), the exonuclease activity of POLγ degrades the 85-mer strand, which leads to the formation of single-stranded DNA (ssDNA) and decreased fluorescent intensity. The assay was performed in a 384-well plate format. Addition of compounds that inhibit POLγ exonuclease activity resulted in a higher fluorescent signal, due to reduced degradation of the 85-mer oligonucleotide.

The exonuclease template used was generated by hybridization of a 100 nucleotides long, single-stranded DNA molecule with an inverted dT at the 3′-end (5′-TTT TTT TTT ATC CGG GCT CGT TAT CTA AGC TGC TCT TGG TAG GCA TTG ACG TCC ATA CTG CAA ATT CAG CTC TGT GCA GTT AGG CAG GAG TCT ACA AGC invT-3′) to an 85 nucleotides long single-stranded DNA molecule (5′-AGC TTG TAG ACT CCT GCC TAA CTG CAC AGA GCT GAA TTT GCA GTA TGG ACG TCA ATG CCT ACC AGA GCA GCT TAG ATA ACG AGC-3′), using a Bio-Rad T100 Thermal Cycler. The inverted dT is characterized by a 3′-3′ linkage, which inhibits both degradation of the 3′-5′ exonuclease activity and extension by DNA polymerases.

POLγ is an heterotrimer of one POLγA subunit (harboring exonuclease and DNA polymerase activities) and two accessory POLγB subunits. Proteins used in the exonuclease assay were POLγA (NP_002684) and POLγB (NP_009146).

The protein mixture used in the assays contained POLγA (1 nM), POLγB (1.3 nM, concentration calculated as a dimer), 25 mM Tris-HCl (pH 8.0), 0.1 mg/mL bovine serum albumin, 1 mM Bond-Breaker TCEP solution (pH 7) [Thermo Fisher], 25 mM NaCl, and 0.02% Triton X-100.

The DNA template mixture used in the assay contained 5 nM of the exonuclease template, 25 mM Tris-HCl (pH 8.0), 10 mM MgCl₂, 0.1 mg/mL bovine serum albumin, 1 mM Bond-Breaker TCEP solution [Thermo Fisher], and 0.02% Triton X-100.

Microplates with compounds (0.1 μL in each well) to be tested in the assay were prepared from 10 mM compound stocks in 100% DMSO, and equal amounts of DMSO without any compound were added to positive and negative control wells.

The protein mixture containing POLγ was dispensed (5 μL in each well) into compound plates and incubated at 37° C. for 15 minutes to allow compound binding to the POLγ complex. After incubation, 5 μL of the DNA template mixture was dispensed into each well. The plates were incubated at 37° C. for 2.5 hours.

During incubation, a combined stop buffer and detection reagent solution was prepared. The buffer contained 25 mM EDTA (pH 8.0), 0.02% Triton X-100, and SYBR Green I [Invitrogen] diluted 10,000× (for fluorescent readout). EDTA will stop the enzymatic reaction by chelating magnesium ions.

After the 2.5-hour incubation period, the SYBR Green I solution was added (10 μL) to the screening plates and the plates were incubated at room temperature in the dark for 20 minutes before the fluorescent signal was read between 485-520 nM on a BMG Pherastar microtiter plate reader. The half-maximal inhibitory concentration (IC₅₀) values are provided in Table 1.

TABLE 1
IC₅₀ Values for POLγ Exonuclease Inhibitors.

| Example | POLγ-Exonuclease |
|---|---|
| No. | IC₅₀ (μM) |
| 1 | 7 |
| 2 | 4 |
| 3 | 10 |
| 4 | 48 |
| 5 | 3 |
| 6 | 12 |
| 7 | 1 |
| 8 | 9 |
| 9 | 2 |
| 10 | 0.8 |
| 11 | >100 |
| 12 | 5 |
| 13 | 6 |
| 14 | 3 |
| 16 | 12 |
| 17 | >100 |
| 18 | 81 |
| 19 | 8 |
| 185 | 6 |
| 186 | 7 |

### DNA Polymerization Assay to Monitor Effects on Human Wildtype POLγ and Four Disease-Causing Mutant Variants of Human POLγ.

The ability of small molecules to stimulate the polymerase activity of POLγ was analyzed in a quantitative fluorescence SYBR Green I assay with fluorescence intensity (FI) readout.

In the presence of dNTPs, wild type human POLγ and mutant derivatives thereof were incubated with single-stranded, circular M13mp18 ssDNA hybridized to a short DNA oligonucleotide that functions as a primer for initiation of DNA synthesis. POLγ and mutant derivatives thereof will extend the primer and use the M13mp18 molecule as a template to synthesize long stretches of double-stranded DNA. SYBR Green I, which is a double-stranded DNA-binding dye, was used to quantify formation of double-stranded DNA in the reaction. When bound to double-stranded DNA, SYBR Green I fluorescence increases by up to 100-fold.

The assay was performed in the 384-well plate format. Upon addition of compounds, changes in double-stranded DNA synthesis activity can be monitored by following effects on fluorescent intensity.

Proteins used in the polymerization assays were wild type POLγA (POLγA:WT) and mutant derivatives thereof in which an Alanine in position 467 of the amino acids sequence had been changed to Threonine (POLγA:A467T), a Glycine in position 848 of the amino acids sequence had been changed to Serine (POLγA:G848S), an Arginine in position 309 of the amino acids sequence had been changed to Cysteine (POLγA:R309C), or a Tyrosine in position 955 of the amino acid sequence had been changed to Cysteine (POLγA:Y955C). Another mutant derivative used in the assay was the W748S mutant, which arises as a result of the substitution of a G to C mutation at position 2243 in exon 13. The W748S mutation is generally found in cis with the E1143G polymorphism, which is caused by an A to G transition at nucleotide 3428 in exon 21 of POLγ. The reactions also contained the accessory POLγB subunit, and the human mitochondrial single-stranded DNA binding protein (mtSSB).

The protein mixture used in the assays contained POLγA or a mutant derivative (1 nM), POLγB (1.3 nM, concentration calculated as a dimer), 25 mM Tris-HCl (pH 8.0), 0.1 mg/mL bovine serum albumin, 1 mM TCEP solution (pH 7), 25 mM NaCl, and 0.02% Triton X-100.

The primed circular ssDNA template used in the polymerization assay was generated by hybridizing circular, single-stranded DNA from M13mp18 with a 20 nucleotides long oligonucleotide (5′-GTA AAA CGA CGG CCA GTG CC-3′) using a Bio-Rad T100 Thermal Cycler.

The DNA template mixture used in the assay contained 0.5 nM primed M13 mp18 ssDNA, 400 nM mtSSB, 0.1 mM Tris-HCl (pH 8.0), 0.1 mM dNTP, 10 mM MgCl2, 0.1 mg/mL BSA, 1 mM TCEP solution, and 0.02% Triton X-100. The DNA template mixture for the wild type protein contained either 100 μM dNTP, 1 μM dNTP, or 0.1 μM dNTP, designated in Table 2 as 100 μM, 1 μM, and 0.1 μM, respectfully.

Microplates with compounds (0.1 μL in each well) to be tested in the assay were prepared from 10 mM compound stocks in 100% DMSO, and equal amounts of DMSO without any compound were added to positive and negative control wells.

The protein mixture was dispensed (5 μL into each well) into compound plates and incubated at 37° C. for 15 minutes. After incubation, 5 μL of the DNA template mixture was dispensed into each well. The plates were then incubated at 37° C. for 2 hours.

During incubation, a combined stop buffer and detection reagent solution was prepared. The buffer contained 25 mM EDTA (pH 8.0), 0.02% Triton X-100, and SYBR Green I [Invitrogen] diluted 10,000× (for fluorescent readout). EDTA will stop the enzymatic reaction by chelating magnesium ions.

After the incubation period, SYBR Green I solution was added (10 μL) to the screening plates and the plates were incubated at room temperature in the dark for 20 minutes before the fluorescent signal was read between 485-520 nM on a BMG Pherastar microtiter plate reader. The compound concentrations for half maximum activity (AC₅₀) are provided in Table 2

TABLE 2
AC₅₀ Values for POLγ Activators on Human Wildtype
POLγ and Human Mutant POLγ.

| | | | | W748S + | | WT | WT | WT |
|---|---|---|---|---|---|---|---|---|
| | A467T | G848S | R309C | E1143G | Y955C | AC₅₀ | AC₅₀ | AC₅₀ |
| Example | AC₅₀ | AC₅₀ | AC₅₀ | AC₅₀ | AC₅₀ | (μM) at | (μM) at | (μM) at |
| No. | (μM) | (μM) | (μM) | (μM) | (μM) | 0.1 μM | 1 μM | 100 μM |
| 1 | 44 | 18 | 12 | | 2 | | | 2 |
| 2 | 60 | 10 | 8 | | 1 | | | 1 |
| 3 | 48 | 31 | 28 | | 5 | | | >100 |
| 4 | 52 | 33 | 25 | | 11 | | | 10 |
| 5 | 33 | 10 | 11 | | 3 | | | 5 |
| 6 | 95 | 30 | 13 | | 5 | | | 4 |
| 7 | 37 | 13 | 10 | | 1 | | | 1 |
| 8 | 39 | 23 | 4 | | 3 | | | 5 |
| 9 | 18 | 6 | 2 | | 2 | | | 2 |
| 10 | 12 | 3 | 1 | 2 | 0.4 | | | 0.5 |
| 11 | 49 | 34 | 6 | 19 | 4 | | | 29 |
| 12 | 45 | 26 | 3 | | 2 | | | 5 |
| 13 | 33 | 10 | 3 | 4 | 2 | | | 1 |
| 14 | 28 | 16 | 13 | | 2 | | | >100 |
| 15 | >100 | 30 | | | | | | >10 |
| 16 | 5 | 2 | 2 | | 0.8 | | | 0.2 |
| 17 | 6 | 6 | 2 | | 0.8 | | | 0.6 |
| 18 | 17 | 8 | 5 | | 3 | | | 1 |
| 19 | 11 | 3 | 3 | | 0.5 | | | 0.8 |
| 20 | 4 | 2 | 1 | 2 | 0.3 | | | 0.3 |
| 21 | 3 | 2 | 1 | 3 | 1 | | | 1 |
| 22 | >100 | >100 | | >100 | | | | |
| 23 | >100 | >100 | | | | | | |
| 24 | >100 | >100 | | | | | | |
| 25 | >100 | >100 | | | | | | |
| 26 | >100 | 50 | | | | | | |
| 27 | >100 | >100 | | | | | | |
| 28 | >100 | >100 | | | | | | |
| 29 | >100 | 33 | | | | 2 | | |
| 30 | >100 | 22 | | | | 2 | | |
| 31 | >100 | >100 | | | | | | |
| 32 | >100 | 35 | | | | 12 | | |
| 33 | >100 | 56 | | | | | | |
| 34 | >100 | 37 | | | | | | |
| 35 | >100 | >100 | | | | | | |
| 36 | >100 | >100 | | | | | | |
| 37 | >100 | >100 | | | | | | |
| 38 | >100 | >100 | | | | | | |
| 39 | >100 | >100 | | | | | | |
| 40 | >100 | >100 | | | | | | |
| 41 | >100 | 21 | | | | >100 | | |
| 42 | >100 | >100 | | | | | | |
| 43 | >100 | 51 | | | | | | |
| 44 | >100 | 27 | | | | | | |
| 45 | | >100 | | | | | | |
| 46 | >100 | 91 | | | | | | |
| 47 | >100 | 39 | | | | | | |
| 48 | >100 | 13 | | 9 | | | | |
| 49 | >100 | 14 | | | | 2 | | |
| 50 | >100 | 11 | | | | 1 | | |
| 51 | >100 | 44 | | | | 6 | | |
| 52 | >100 | >100 | | | | | | |
| 53 | >100 | 63 | | | | | | |
| 54 | >100 | 21 | | | | 1 | | |
| 55 | >100 | >100 | | | | | | |
| 56 | >100 | 56 | | | | | | |
| 57 | >100 | >100 | | | | | | |
| 58 | >100 | 46 | | | | 3 | | |
| 59 | >100 | >100 | | | | | | |
| 60 | 5 | 2 | 0.9 | | 0.4 | | | 0.6 |
| 61 | >100 | >100 | | 0.7 | | | | |
| 62 | >100 | >100 | | >100 | | | | |
| 63 | >100 | >100 | | >100 | | | | |
| 64 | 8 | 1 | 1 | 4 | >100 | | | 0.7 |
| 65 | 15 | 12 | 5 | 7 | 2 | | | 8 |
| 66 | 11 | 1 | 12 | 5 | 2 | | | 0.5 |
| 67 | >100 | | | 21 | | | | >10 |
| 68 | 16 | | 7 | 2 | 2 | | | 0.2 |
| 69 | 17 | 14 | 1 | | 0.4 | | | 1 |
| 70 | 32 | 49 | 51 | | 7 | | | 11 |
| 71 | >100 | >100 | | | | | | |
| 72 | 37 | 98 | 30 | | 6 | | | 4 |
| 73 | 16 | 14 | 1 | | 0.8 | | | 2 |
| 74 | >100 | >100 | | | | | | |
| 75 | >100 | >100 | | | | | | |
| 76 | 3 | 3 | 6 | 6 | 2 | | | 0.4 |
| 77 | >100 | >100 | | | | | | |
| 78 | >100 | >10 | | | | | | |
| 79 | >100 | >10 | | | | | | |
| 80 | >100 | >10 | >100 | >100 | >100 | >100 | | |
| 81 | 3 | 2 | 0.6 | 0.7 | 0.2 | | | 0.2 |
| 82 | >100 | >10 | | | | | | |
| 83 | >100 | 3 | | | | | | 6 |
| 84 | >100 | >10 | | | | | | |
| 85 | >100 | >100 | | | | | | |
| 86 | >100 | >100 | | | | | | |
| 87 | 10 | 14 | 6 | 5 | 6 | | | 4 |
| 88 | >100 | >100 | | | | | | |
| 89 | 8 | 3 | 4 | 0.8 | 0.1 | | | 0.3 |
| 90 | >100 | >100 | | | | | | |
| 91 | >100 | >100 | | | | | | |
| 92 | >100 | 1 | | | | | | >10 |
| 93 | >100 | 9 | | | | | | 3 |
| 94 | >100 | >100 | | | | | | |
| 95 | >100 | >100 | | | | | | |
| 96 | 24 | 4 | >100 | 2 | 50 | | | >10 |
| 97 | >100 | >100 | | | | | | |
| 98 | >100 | 9 | >100 | >100 | >10 | | | >10 |
| 99 | >100 | >100 | | | | | | |
| 100 | >100 | >100 | | | | | | |
| 101 | >100 | >100 | | | | | | |
| 102 | >100 | 0.4 | >100 | 10 | >100 | 5 | | >10 |
| 103 | >100 | 9 | 58 | >100 | 9 | 17 | | 1 |
| 104 | >100 | 47 | | | | | | >10 |
| 105 | >100 | 9 | | | | | | >10 |
| 106 | >100 | >100 | | | | | | 32 |
| 107 | 28 | 7 | | | | | | 1 |
| 108 | >100 | >100 | | | | | | 24 |
| 109 | >100 | >100 | | | | | | >100 |
| 110 | >100 | >100 | | | | | | >100 |
| 111 | >100 | >100 | | | | | | 3 |
| 112 | >100 | 40 | | | | | | 20 |
| 113 | >100 | 11 | | | | | | |
| 114 | >100 | 50 | | | | | | |
| 115 | >100 | >100 | | | | | | |
| 116 | >100 | 9 | | | | | | >10 |
| 117 | >100 | >100 | | | | | | |
| 118 | >100 | >100 | | | | | | |
| 119 | >100 | 2 | | | | | | >10 |
| 120 | >100 | >100 | | | | | | |
| 121 | >100 | >100 | | | | | | |
| 122 | >100 | >100 | | | | | | |
| 123 | >100 | 2 | | | | | | 3 |
| 124 | >100 | 2 | | | | | | 1 |
| 125 | 8 | 7 | | | | | | >10 |
| 126 | 32 | >100 | | | | | | |
| 127 | >100 | >100 | | | | | | |
| 128 | >100 | >100 | | | | | | |
| 129 | >100 | >100 | | | | | | |
| 130 | >100 | >100 | | | | | | |
| 131 | >100 | 3 | | | | | | 1 |
| 132 | >100 | >100 | | | | | | |
| 133 | >100 | 18 | | | | | | |
| 134 | 3 | 1 | | | | | | 0.3 |
| 135 | 32 | 2 | | | | | | 0.6 |
| 136 | 5 | 2 | | | | | | 0.8 |
| 137 | >100 | 11 | | | | | | |
| 138 | >100 | >100 | | | | | | |
| 139 | >100 | >100 | | | | | | |
| 140 | >100 | >100 | | | | | | |
| 141 | >100 | >100 | | | | | | |
| 142 | >100 | >100 | | | | | | >10 |
| 143 | >100 | >100 | | | | | | >10 |
| 144 | >100 | >100 | | | | | | >10 |
| 145 | >100 | 67 | | | | | | |
| 146 | >100 | >100 | | | | | | |
| 147 | >100 | >100 | | | | | | |
| 148 | >100 | >100 | | | | | | |
| 149 | >100 | >100 | | | | | | |
| 150 | >100 | >100 | | | | | | |
| 151 | >100 | 2 | | | | | | >10 |
| 152 | >100 | 6 | | | | | | >10 |
| 153 | >100 | >100 | | | | | | |
| 154 | >100 | >100 | | | | | | |
| 155 | >100 | 4 | | | | | 0.4 | |
| 156 | >100 | >100 | | | | | | |
| 157 | >100 | >100 | | | | | | |
| 158 | >100 | >100 | | | | | | |
| 159 | >100 | >100 | | | | | | |
| 160 | >100 | 14 | | | | | | |
| 161 | >100 | 31 | | | | | | |
| 162 | 91 | 13 | | | | | | 0.4 |
| 163 | >100 | 37 | | | | | | |
| 164 | 0.6 | 0.4 | | | | | | 0.3 |
| 165 | 1 | | | | | | | 0.7 |
| 166 | 2 | 0.6 | | 0.4 | | | | 2 |
| 167 | 8 | 2 | | 2 | | | | 4 |
| 168 | >100 | >100 | | >100 | | | | >10 |
| 169 | >100 | >100 | | >100 | | | | >10 |
| 170 | >10 | >10 | | >10 | | | | >10 |
| 171 | >10 | >10 | | >10 | | | | 5 |
| 172 | >10 | >10 | | >100 | | | | >100 |
| 173 | 2 | 2 | | 0.4 | | | | 0.2 |
| 174 | 2 | 0.4 | | 0.7 | | | | 0.1 |
| 175 | 3 | 1 | | 10 | | | | 3 |
| 176 | >100 | 0.4 | | 0.3 | | | | >100 |
| 177 | >100 | >100 | | >100 | | | | >100 |
| 178 | >100 | 1 | | 1 | | | | >100 |
| 179 | >100 | >10 | | >10 | | | | >10 |
| 180 | >10 | >10 | | >10 | | | | >10 |
| 181 | >10 | 0.7 | | >10 | | | | >10 |
| 182 | | 0.9 | | 1 | | | | |
| 183 | 6 | 0.5 | | 0.7 | | | | |
| 184 | 33 | 24 | 8 | | 2 | | | >100 |
| 185 | 55 | 15 | 8 | | 2 | | | 6 |

What is claimed is:

1. A compound, or a pharmaceutically acceptable salt thereof, according to formula (I): 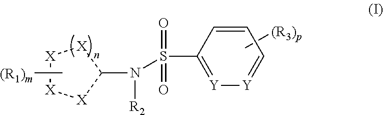
wherein:
dashed lines are independently single or double bonds;
R¹ is selected from the group consisting of:
C₁-C₄ alkyl,
halogen,
C(O)-phenyl,
C(O)—NR⁴R⁵,
C(O)-pyrrolidine optionally substituted with one or more C₁-C₄ alkyl,
tetrahydropyran optionally substituted with one or more C₁-C₄ alkyl,
phenyl optionally substituted with one or more C₁-C₄ alkyl or —OR⁶,
pyridine optionally substituted with one or more C₁-C₄ alkyl,
thiazole optionally substituted with a group selected from the group consisting of:
C₁-C₄ alkyl,
C₃-C₆ cycloalkyl,
R⁶—OH,
R⁶—NR⁶R⁶,
CN,
C(O)—OH,
C(O)—OR⁶,
C(O)—NH₂,
C(O)—NHR⁶,
C(O)—NR⁶R⁶, and
tetrazole optionally substituted with one or more C₁-C₄ alkyl,
oxazole optionally substituted with one or more C₁-C₄ alkyl or C₃-C₇ cycloalkyl,
pyrazole optionally substituted with one or more C₁-C₄ alkyl or C₃-C₇ cycloalkyl,
imidazole optionally substituted with one or more C₁-C₄ alkyl or C₃-C₇ cycloalkyl, wherein the C₁-C₄ alkyl is optionally substituted with phenyl, and
piperidine optionally substituted with one or more of C₁-C₄ alkyl or C(O)—R⁶,
or two R¹ are joined together to form a 6-membered aryl ring, a 5-membered saturated heterocyclic ring, or a 5-membered unsaturated heterocyclic ring, wherein the aryl ring is optionally substituted with one or more of C₁-C₄ alkyl or one or more halogen, and wherein either heterocyclic ring optionally contains a second heteroatom selected from O and S, and either heterocyclic ring is optionally substituted with one or more groups selected from the group consisting of C₁-C₄ alkyl, phenyl, and oxo,
or two R¹ are joined together to form a phenyl group that is optionally substituted with one or more of C₁-C₄ alkyl or halogen;
R² is hydrogen or C₁-C₄ alkyl;
R³ is selected from the group consisting of C₁-C₄ alkyl, C₁-C₄ alkoxy, C₁-C₄ trihaloalkyl, CN, and halogen,
or two R³ are joined together to form a 5- to 6-membered unsaturated heterocyclic ring that optionally contains a second heteroatom selected from N and S;
R⁴ and R⁵ are each independently selected from the group consisting of H, C₁-C₄ alkyl, 4- to 6-membered heterocyclic ring containing O, 4- to 6-membered cycloalkyl, and 4to 6-membered heteroaryl, wherein the C₁-C₄ alkyl is optionally substituted with a group selected from the group consisting of C₃-C₇ cycloalkyl, OH, OR⁶, phenyl, C(O)—NH₂, and C(O)—NR⁶R⁶,
or R⁴ and R⁵ together with the nitrogen atom to which they are joined form a 5- to 7-membered heterocyclic ring optionally containing a second heteroatom selected from N, O, and S, wherein the heterocyclic ring is optionally substituted with one or more groups independently selected from the group consisting of C₁-C₄ alkyl, C₃-C₇ cycloalkyl, C₁-C₄ alkoxy, halogen, hydroxy, NH₂, NHR⁶, NR⁶R⁶, cyano, C(O)—R⁶, C(O)NH₂, wherein the C₁-C₄ alkyl is optionally substituted with OH or OR⁶,
or R⁴ and R⁵ together with the nitrogen atom to which they are joined form a 7- to 9-membered heterobicyclic ring optionally containing one or two additional N atoms, and optionally substituted with C₁-C₄ alkyl;
R⁶ is C₁-C₄ alkyl;
X is selected from the group consisting of C, CH, CH₂, O, N, NR, and S, as allowed by valency;
Y is C, CH or N, as allowed by valency;
m is 1-2;
n is 1-2; and
p is 0-3.

2. The compound, or a pharmaceutically acceptable salt thereof, according to claim 1 wherein:
dashed lines are independently single or double bonds;
R¹ is selected from the group consisting of:
C₁ alkyl,
F,
C(O)-phenyl,
C(O)—NR⁴R⁵,
C(O)-pyrrolidine optionally substituted with C₁ alkyl,
tetrahydropyran,
phenyl optionally substituted with methoxy,
pyridine,
thiazole optionally substituted with a group selected from the group consisting of:
C₁ alkyl,
C₃ cycloalkyl,
R⁶—OH,
R⁶—NR⁶R⁶,
CN,
C(O)—OH,
C(O)—OR⁶,
C(O)—NH₂,
C(O)—NHR⁶,
C(O)—NR⁶R⁶, and
tetrazole,
oxazole optionally substituted with C₃ cycloalkyl,
pyrazole optionally substituted with C₁ alkyl or C₃ cycloalkyl,
imidazole optionally substituted with one or more of C₁ alkyl or C₃ cycloalkyl, wherein the C₁ alkyl is optionally substituted with phenyl, and
piperidine optionally substituted with C(O)—R⁶,
or two R¹ are joined together to form a 6-membered aryl ring, a 5-membered saturated heterocyclic ring, or a 5-membered unsaturated heterocyclic ring, wherein the aryl ring is optionally substituted with chloro, and wherein either heterocyclic ring optionally contains a second heteroatom selected from O and S, and either heterocyclic ring is optionally substituted with one or more groups selected from the group consisting of C₁-C₃ alkyl, phenyl, and oxo,
or two R¹ are joined together to form a phenyl group that is optionally substituted with Cl;
R² is hydrogen;
R³ is selected from the group consisting of C₁ alkyl, C₁ alkoxy, trifluoromethyl, chloro, fluoro,
or two R³ are joined together to form a 5- to 6-membered unsaturated heterocyclic ring that optionally contains a second heteroatom selected from N and S;
R⁴ and R⁵ are each independently selected from the group consisting of H, C₁-C₃ alkyl, 4-membered heterocyclic ring containing O, 5- or 6-membered cycloalkyl, and 6-membered heteroaryl, wherein the C₁-C₃ alkyl is optionally substituted with a group selected from the group consisting of C₃-cycloalkyl, OH, OR⁶, phenyl, and C(O)—NH₂,
or R⁴ and R⁵ together with the nitrogen atom to which they are joined form a 5- to 7-membered heterocyclic ring optionally containing a second heteroatom selected from N, O, and S, wherein the heterocyclic ring is optionally substituted with one or more groups independently selected from the group consisting of C₁-C₃ alkyl, C₃ cycloalkyl, halogen, NH₂, NHR⁶, NR⁶R⁶, cyano, C(O)—C₁-C₃ alkyl, C(O)NH₂, wherein the C₁-C₃ alkyl is optionally substituted with OH,
or R⁴ and R⁵ together with the nitrogen atom to which they are joined form a 7- to 9-membered heterobicyclic ring optionally containing one or two additional N atoms, and optionally substituted with C₁ alkyl;
R⁶ is C₁ alkyl;
X is selected from the group consisting of C, CH, CH₂, O, N, NR, and S, as allowed by valency;
Y is C, CH or N, as allowed by valency;
m is 1-2;
n is 1-2; and
p is 0-3.

3. The compound, or a pharmaceutically acceptable salt thereof, according to claim 2 wherein R¹ is selected from the group consisting of:
C₁ alkyl,
F,
C(O)—NR⁴R⁵,
C(O)-pyrrolidine optionally substituted with C₁ alkyl,
phenyl optionally substituted with methoxy,
thiazole optionally substituted with a group selected from the group consisting of:
C₁ alkyl,
C₃ cycloalkyl,
R⁶—OH,
R⁶—NR⁶R⁶,
CN,
C(O)—OH,
C(O)—OR⁶,
C(O)—NH₂,
C(O)—NHR⁶,
C(O)—NR⁶R⁶, and
tetrazole, and
pyrazole optionally substituted with C₁ alkyl or C₃ cycloalkyl.

4. The compound, or a pharmaceutically acceptable salt thereof, according to claim 2 wherein the group 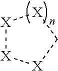
is selected from the group consisting of: 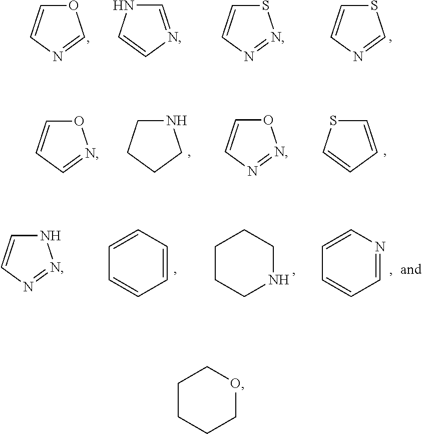
as allowed by valency.

5. The compound, or a pharmaceutically acceptable salt thereof, according to claim 4 wherein the group 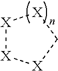
is selected from the group consisting of: 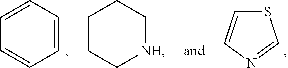
as allowed by valency.

6. The compound, or a pharmaceutically acceptable salt thereof, according to claim 2 wherein the group 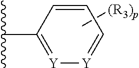
is selected from the group consisting of:

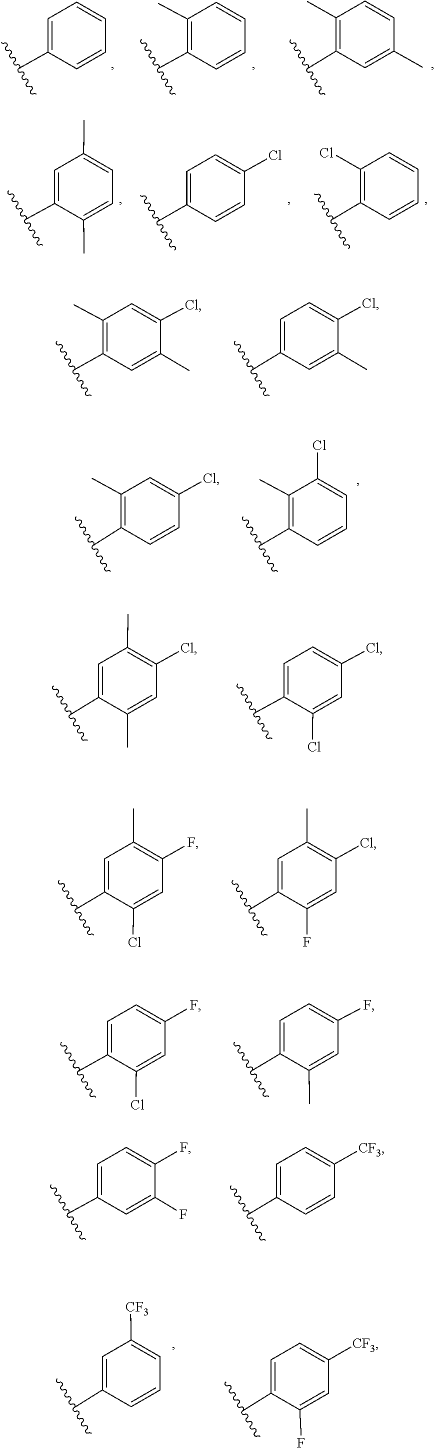

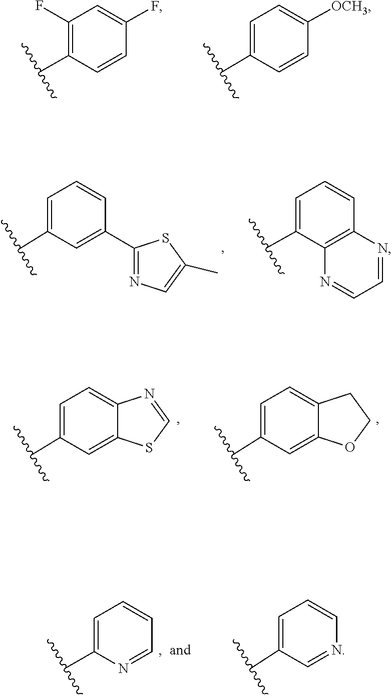

7. The compound, or a pharmaceutically acceptable salt thereof, according to claim 6 wherein the group

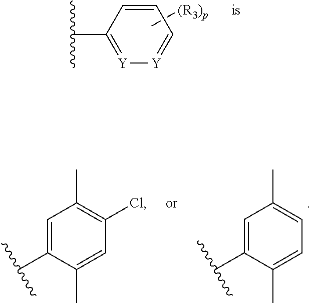

8. The compound, or a pharmaceutically acceptable salt thereof, according to claim 2 wherein R¹ is C(O)—NR⁴R⁵ and is selected from the group consisting of:

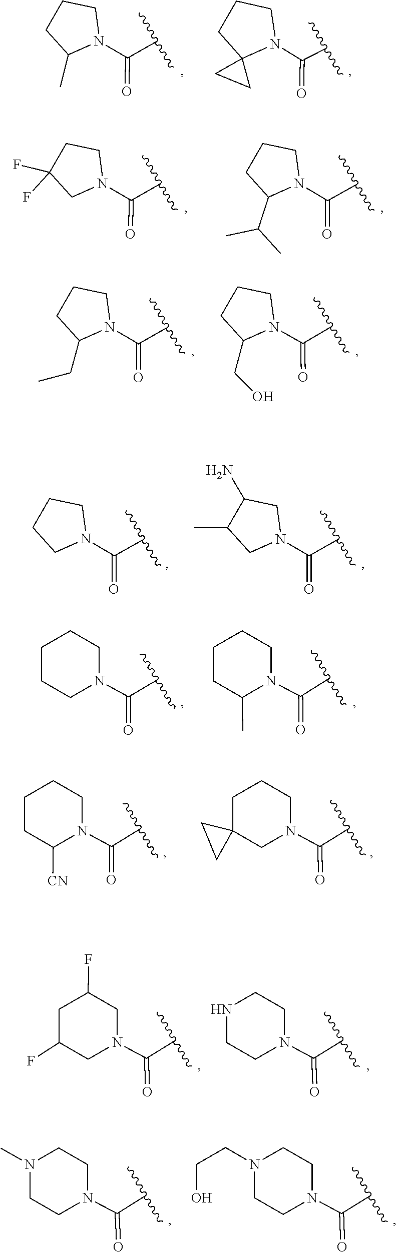

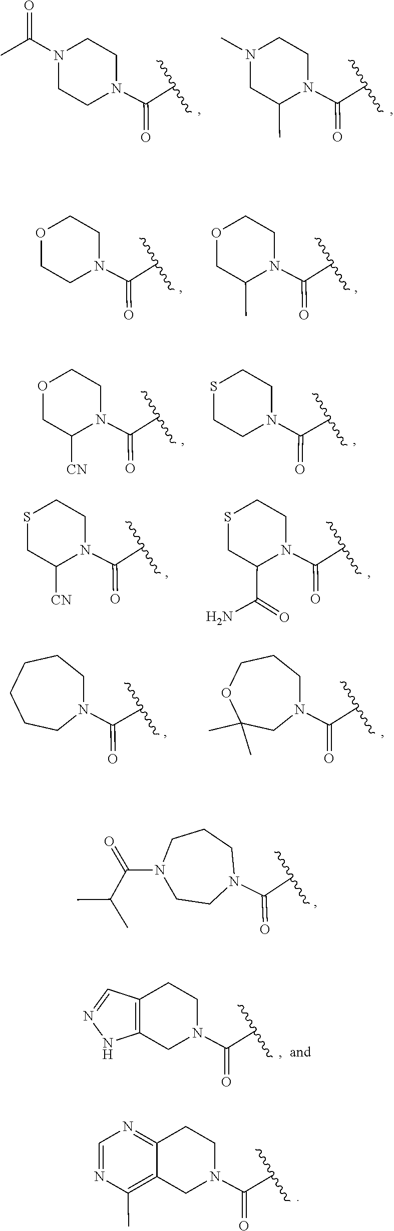

9. The compound, or a pharmaceutically acceptable salt thereof, according to claim 8 wherein R¹ is C(O)—NR⁴R⁵ and is selected from

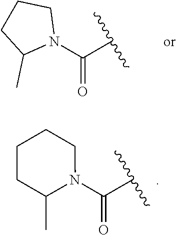

10. The compound, or a pharmaceutically acceptable salt thereof, according to claim 1 wherein the compound is selected from the group consisting of:

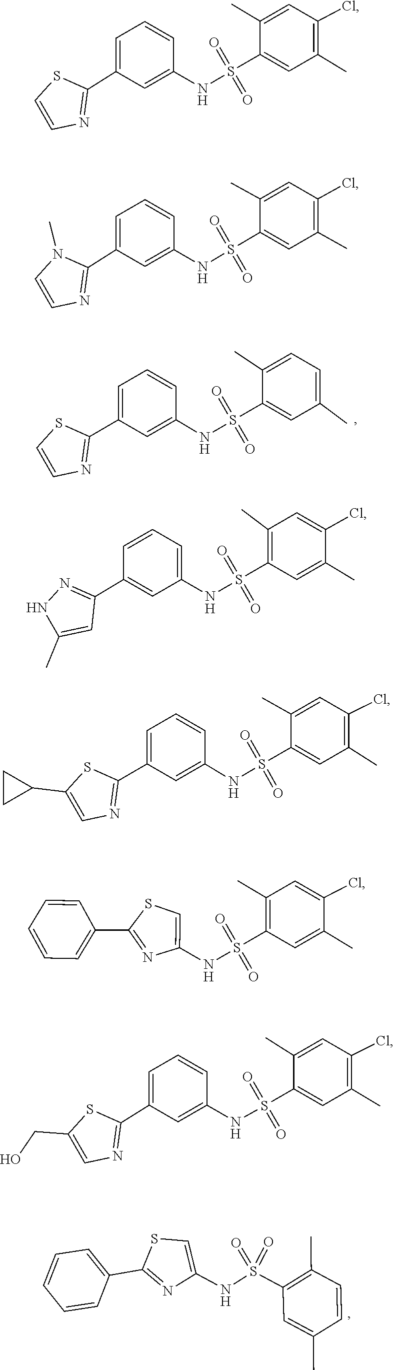

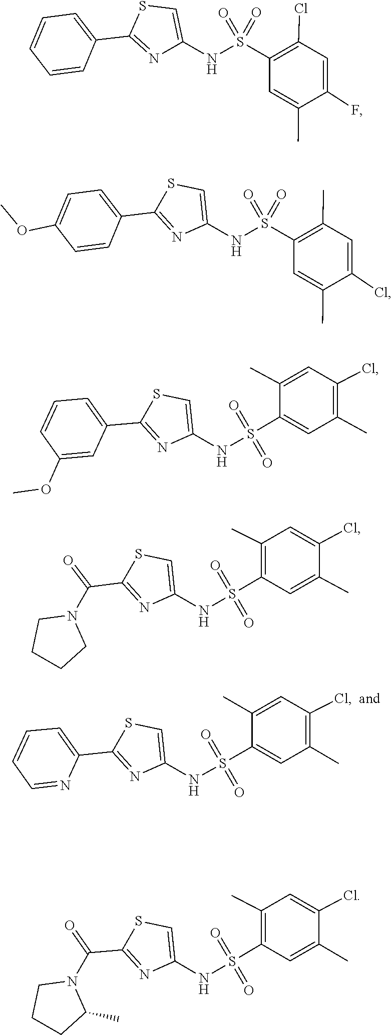

11. The compound, or a pharmaceutically acceptable salt thereof, according to claim 10 wherein the compound is selected from the group consisting of:

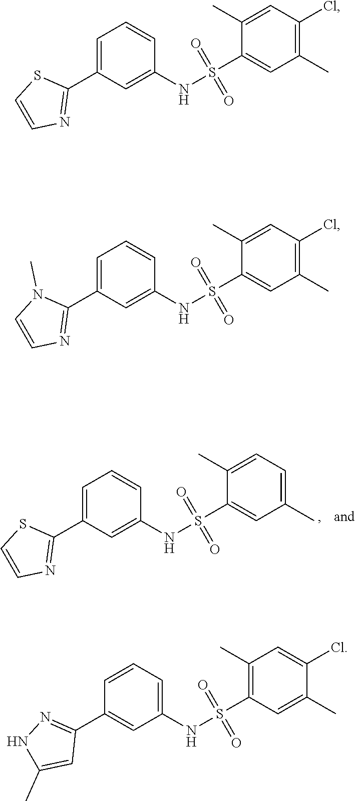 [SMILES: Cc1cc(S(=O)(=O)Nc2cccc(-c3nccs3)c2)c(C)cc1Cl ; Cc1cc(S(=O)(=O)Nc2cccc(-c3nccn3C)c2)c(C)cc1Cl ; Cc1ccc(C)c(S(=O)(=O)Nc2cccc(-c3nccs3)c2)c1 ; Cc1cc(-c2cccc(NS(=O)(=O)c3cc(C)c(Cl)cc3C)c2)n[nH]1]

12. The compound, or a pharmaceutically acceptable salt thereof, according to claim 10 wherein the compound is selected from the group consisting of:

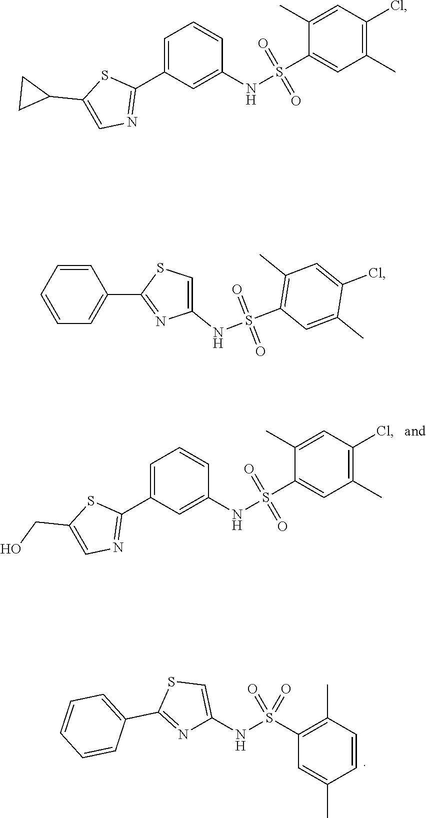 [SMILES: Cc1cc(S(=O)(=O)Nc2cccc(-c3ncc(C4CC4)s3)c2)c(C)cc1Cl ; Cc1cc(S(=O)(=O)Nc2csc(-c3ccccc3)n2)c(C)cc1Cl ; Cc1cc(S(=O)(=O)Nc2cccc(-c3ncc(CO)s3)c2)c(C)cc1Cl ; Cc1ccc(C)c(S(=O)(=O)Nc2csc(-c3ccccc3)n2)c1]

13. The compound, or a pharmaceutically acceptable salt thereof, according to claim 10 wherein the compound is selected from the group consisting of:

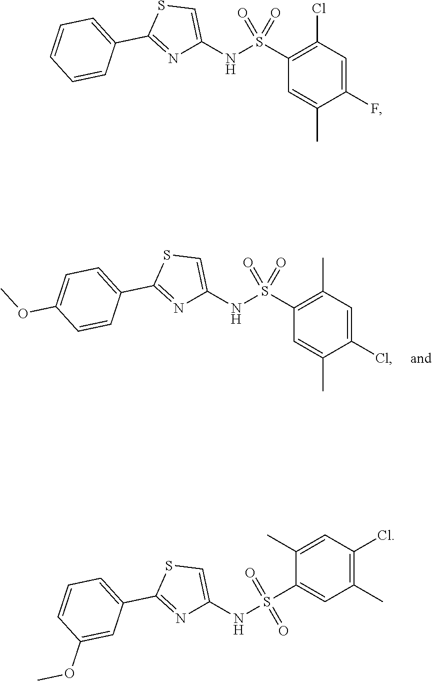

14. The compound, or a pharmaceutically acceptable salt thereof, according to claim 10 wherein the compound is selected from the group consisting of:

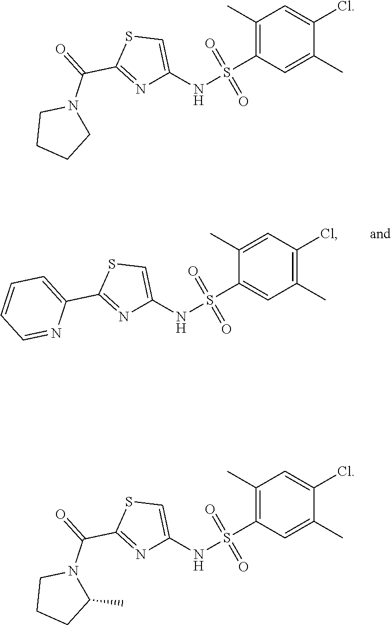 [SMILES: Cc1cc(S(=O)(=O)Nc2csc(C(=O)N3CCCC3)n2)c(C)cc1Cl ; Cc1cc(S(=O)(=O)Nc2csc(-c3ccccn3)n2)c(C)cc1Cl ; Cc1cc(S(=O)(=O)Nc2csc(C(=O)N3CCC[C@H]3C)n2)c(C)cc1Cl]

15. A pharmaceutical composition comprising a compound according to claim 1, and a pharmaceutically acceptable excipient.

16. A pharmaceutical composition comprising a compound according to claim 2, and a pharmaceutically acceptable excipient.

17. A pharmaceutical composition comprising a compound according to claim 10, and a pharmaceutically acceptable excipient.

18. A method of modulating POLγ with a compound according to claim 1, or a pharmaceutically acceptable salt thereof.

19. A method of modulating POLγ with a compound according to claim 2, or a pharmaceutically acceptable salt thereof.

20. A method of modulating POLγ with a compound according to claim 10, or a pharmaceutically acceptable salt thereof.
